(19) United States
(12) Patent Application Publication
(10) Pub. No.: US 2024/0216900 A1
(43) Pub. Date: Jul. 4, 2024

(54) SABRE CATALYSTS CONTAINING FLUORINATED CARBON CHAINS FOR DELIVERY OF METAL-FREE MRI CONTRAST AGENTS

(71) Applicant: The U.S.A., as represented by the Secretary, Department of Health and Human Services, Bethesda, MD (US)

(72) Inventors: Rolf E. Swenson, Silver Spring, MD (US); Jessica H. Ettedgui-Benjamini, North Bethesda, MD (US); Carolyn Woodroofe Hitko, Olney, MD (US); Murali K. Cherukuri, Potomac, MD (US); Natarajan Raju, Germantown, MD (US)

(73) Assignee: The U.S.A., as represented by the Secretary, Department of Health and Human Services, Bethesda, MD (US)

(21) Appl. No.: 18/410,773

(22) Filed: Jan. 11, 2024

Related U.S. Application Data
(63) Continuation-in-part of application No. PCT/US2023/017885, filed on Apr. 7, 2023.
(60) Provisional application No. 63/328,545, filed on Apr. 7, 2022.

(51) Int. Cl. B01J 31/22; C07C 51/487

(52) U.S. Cl. CPC B01J 31/2295; B01J 31/226; B01J 31/2273; C07C 51/487; B01J 2231/005; B01J 2531/004; B01J 2531/827

(57) ABSTRACT

Disclosed are perfluorinated SABRE catalysts comprising a d-block element and a perfluorinated ligand, wherein the perfluorinated ligand is of Formula (I): [Lₘ-(NHC)—(Y—Z)_{q}] or a salt thereof. Also disclosed is a method of preparing a hyperpolarized substrate comprising a ½ spin nucleus or nuclei using the perfluorinated SABRE catalysts, and isolating the resulting hyperpolarized substrate for administration to an animal. Further disclosed is a method of imaging a tissue of an animal suspected of having a disease or condition.

### CROSS-REFERENCE TO PRIOR APPLICATIONS

This application is a continuation-in-part of International Patent Application No. PCT/US2023/017885, filed Apr. 7, 2023, which claims benefit to U.S. Provisional Patent Application No. 63/328,545, filed Apr. 7, 2022, which are hereby incorporated by reference in their entireties.

### FEDERALLY SPONSORED RESEARCH OR DEVELOPMENT

This invention was made with Government support and the Government has certain rights in this invention.

### BACKGROUND OF THE INVENTION

Nuclear magnetic resonance spectroscopy (NMR) and magnetic resonance imaging (MRI) methods are powerful tools widely used in biomedical, chemical, and materials science applications. These methods rely on the population difference of nuclear spin energy levels (called polarization) created after applying a strong magnetic field. Spins aligned with or against the applied field produce a net polarization, which is detected. Unfortunately, the nuclear polarization at thermal equilibrium (i.e., normal conditions) is inherently poor and remains a limitation to sensitivity and scope of the capabilities of magnetic resonance in general (Gunther, NMR Spectrosc. Basic Princ. Concepts i Appl. Chem., 13-28 (2013)).

Hyperpolarization techniques have been developed to overcome this problem and allow orders of magnitude NMR/MRI signal enhancement. The most widely used hyperpolarization techniques employ polarization transfer from electrons (dynamic nuclear polarization, DNP) (Hausser et al., Adv. Magn. Opt. Reson., 3, 79-139 (1968); Abagam et al., Reports Prog. Phys., 41, 395-467 (1978); and Ardenkjaer-Larsen et al., Proc. Natd. Acad. Sci. U.S.A, 100, 10158-10163 (2003)), photons (spin exchange optical pumping) (Bhaskar et al., Phys. Rev. Lett., 49, 25 (1982); Ebert et al., Lancet, 347, 1297 (1996); Albert et al., Nature, 370, 199-201 (1994); and Schroder et al., Science, 314, 446-449 (2006)), or parahydrogen (parahydrogen-induced polarization, PHIP) (Bowers et al., Phys. Rev. Lett., 57, 2645 (1986); Bowers et al., J Am. Chem. Soc., 109, 5541-5542 (1987); Eisenschmid et al., J Am. Chem. Soc., 109, 8089 (1987); Haake et al., J Am. Chem. Soc., 118, 8688 (1996); Goldman et al., C. R. Phys., 6, 575 (2005); and Chekmenev et al., J Am. Chem. Soc., 130, 4212 (2008)). Hyperpolarized magnetic resonance (MR) is an emerging molecular imaging method to monitor metabolism, enzymatic conversions, or biochemical pathways, previously inaccessible using MR.

Current hyperpolarized imaging with dissolution DNP and superconducting MRI scanners is very powerful because of its unique ability to track chemical transformations in vivo. However, DNP based experiments are relatively burdensome, slow, and expensive.

The PHIP approach and its subcategory SABRE (signal amplification by reversible exchange) allow the transfer of the 100% pure singlet spin order of parahydrogen (para-H₂) into a target molecule. The PHIP method is a traditional hydrogenative method and relies on a catalytic hydrogenation reaction where a precursor, in the form of a hydrogen acceptor, is reduced by the parahydrogen and polarized. In contrast, the reversible exchange using SABRE leaves the hyperpolarized agent chemically unchanged. It is also not limited to one para-H₂ molecule per molecule and therefore multiple spin transfer steps can lead to impressive levels of hyperpolarization. This effect has also been shown to transfer polarization to nucleis such as ¹H, ¹³C, ¹⁹F, ³¹P and ¹⁵N and/or ²⁹Si nuclei in a wide range of biologically relevant molecules (Barskiy et al., ChemPhysChem, 18, 1493-1498 (2017); Theis et al., J. Am. Chem. Soc., 137, 1404-1407 (2015); Shchepin et al., ChemPhysChem, 18, 1961-1965 (2017); Zhivonitko et al., Chem. Commun., 51, 2506-2509 (2015); Iali et al., Angew. Chemie—Int. Ed., 58, 10271-10275 (2019); and Gemeinhardt et al., Angew. Chemie Int. Ed., 59, 418-423 (2019)).

High polarization percentage, short signal build-up times, low cost, and scalability make SABRE a promising modality for studying metabolism in vivo using magnetic resonance spectroscopy technique.

The presently available hyperpolarized contrast agents come associated with a spin transfer catalyst component which contains a heavy metal, e.g., a transition metal atom, necessary to enable polarization transfer from para-H₂ to the substrate. Toxicity concerns are raised when the hyperpolarization contrast agents are administered in vivo due to the presence of potentially toxic heavy metal-based complexes (e.g., catalysts are typically Ir-based organometallic compounds) in solution along with hyperpolarized contrast agents.

Another obstacle to the successful implementation of the SABRE process is the lower solubility of parahydrogen (H₂ solubility in water is about 1.6 mg/L) in water compared to that in alcohol-based solvents. The SABRE hyperpolarization is mostly active in organic solvents, preferably methanol, which is not compatible with in vivo administration.

The foregoing shows that there exists a need for improved SABRE catalysts that are easily separable from a hyperpolarized substrate such that the hyperpolarized substrate is free of a heavy metal. There further exists a need for a method for separating a hyperpolarized substrate from the SABRE catalyst and/or hyperpolarized SABRE catalyst complex containing a heavy metal. There also exists a need for a method of administering a hyperpolarized substrate in a solvent medium that is suitable for in vivo administration.

The invention provides such SABRE catalysts and methods. These and other advantages of the invention, as well as additional inventive features, will be apparent from the description of the invention provided herein.

### BRIEF SUMMARY OF THE INVENTION

The present invention provides a perfluorinated SABRE catalyst comprising a d-block element and a perfluorinated ligand, wherein the perfluorinated ligand is of Formula (I):

[Lₘ-(NHC)—(Y—Z)_{q}] Formula (I),
or a salt thereof, and wherein
   each L is independently selected from hydrogen, adamantyl, a substituted or unsubstituted aromatic group, or a substituted or unsubstituted heteroaromatic group,
   NHC is a 4 to 7-membered N-heterocyclic carbenyl group where NHC is bound to the d-block element via a carbene,
   each Y is independently selected from a bond or a spacer group,
   each Z is a perfluorinated tag,
   m is an integer from 1 to 4, and
   q is an integer from 1 to 3.

The present invention also provides a method of preparing the perfluorinated SABRE catalyst described herein, comprising reacting a perfluorinated compound with a base to form a carbene, and reacting the carbene with [(d-block element)(COD)Cl]₂, wherein COD stands for 1,4-cyclooctadienyl.

The present invention further provides a method of preparing a hyperpolarized substrate, the method comprising:
(i) providing a perfluorinated SABRE catalyst comprising a d-block element and a perfluorinated ligand, wherein the perfluorinated ligand is of Formula (I):

[Lₘ-(NHC)—(Y—Z)_{q}] Formula (I),
or a salt thereof, and wherein
   each L is independently selected from hydrogen, adamantyl, a substituted or unsubstituted aromatic group, or a substituted or unsubstituted heteroaromatic group,
   NHC is a 4 to 7-membered N-heterocyclic carbenyl group where NHC is bound to the d-block element via a carbene,
   each Y is independently selected from a bond or a spacer group,
   each Z is a perfluorinated tag,
   m is an integer from 1 to 4, and
   q is an integer from 1 to 3;
   (ii) providing a co-ligand to interact with the perfluorinated SABRE catalyst to facilitate formation of an active perfluorinated SABRE catalyst;
   (iii) combining the active perfluorinated SABRE catalyst with parahydrogen and a substrate comprising a ½ spin nucleus or nuclei in a solvent to obtain a first reaction mixture;
   (iv) hyperpolarizing the first reaction mixture obtained in (iii) by exposing the mixture to a magnetic field or by radiofrequency excitation to obtain a hyperpolarized active perfluorinated SABRE catalyst-substrate and/or a hyperpolarized substrate;
   (v) performing an aqueous phase extraction and/or a fluorinated phase extraction to separate the hyperpolarized substrate from the perfluorinated SABRE catalyst;
   (vi) providing additional co-ligand to interact with the perfluorinated SABRE catalyst to reactivate the perfluorinated SABRE catalyst;
   (vii) combining the reactivated perfluorinated SABRE catalyst with additional parahydrogen and additional substrate comprising a ½ spin nucleus or nuclei in the solvent to obtain a second reaction mixture; and
   (viii) hyperpolarizing the second reaction mixture obtained in (vii) by exposing the mixture to a magnetic field or by radiofrequency excitation to obtain additional hyperpolarized active perfluorinated SABRE catalyst-substrate and/or additional hyperpolarized substrate.

The present invention further provides a hyperpolarized substrate obtained from a method described therein, a pharmaceutically acceptable salt thereof, or a pharmaceutical composition comprising the same.

The present invention further provides a method of obtaining a magnetic resonance image of a tissue in a subject having or suspected to have a cancer or an adverse vascular condition comprising administering to the subject a hyperpolarized substrate described herein, or a pharmaceutical composition comprising the same, and imaging the subject by magnetic resonance imaging.

The present invention further provides a perfluorinated compound of Formula (III):

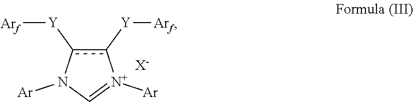

wherein each Ar is independently selected from a substituted or unsubstituted aromatic group or a substituted or unsubstituted heteroaromatic group,
each Ar_{f} is independently selected from a perfluorinated substituted or unsubstituted aromatic group or a perfluorinated substituted or unsubstituted heteroaromatic group,
each Y is independently selected from a bond or a spacer group,
X is an anion, and
[symbol] is a single bond or a double bond. The invention also provides a method of preparing a perfluorinated compound of Formula (III).

### BRIEF DESCRIPTION OF THE DRAWINGS

FIG. 1 illustrates a fluorous solid phase extraction (F-SPE) process wherein an organic fraction containing a hyperpolarized substrate is separated from the fluorous fraction on a fluorous silica gel with a fluorophobic solvent, or a fluorophobic pass, followed by recovering the fluorous fraction, which contains the perfluorinated SABRE catalyst with aqueous/organic solvents, or a fluorophilic pass, in accordance with an aspect of the invention.

FIG. 2 illustrates the substrate polarization process and its extraction from the SABRE catalyst from the fluorous silica gel illustrated in FIG. 1 by a fluorophilic pass, for example, using methanol or ethanol, in accordance with an aspect of the invention.

FIG. 3 illustrates a ‘reverse’ F-SPE process wherein the fluorous fraction is separated from the aqueous/organic fraction on a standard silica gel with a fluorophilic solvent, or fluorophilic pass, in accordance with an aspect of the invention. The hyperpolarized substrate present in the organic fraction is recovered from the silica gel by extraction with a standard organic solvent, or by a fluorophobic pass.

FIG. 4 illustrates the partitioning of the perfluorinated SABRE catalyst and the hyperpolarized substrate between two immiscible phases (fluorous solvent fraction and water or another water immiscible solvent such as chlorinated solvents and water or other hydrophilic solvent(s)) in accordance with an aspect of the invention, which allows the principles of phase-transfer catalysis to be employed in conjunction with parahydrogen to produce high levels of hyperpolarization in the aqueous phase without catalyst contamination.

FIG. 5 illustrates the hyperpolarization of a fluorinated SABRE catalyst containing a metal and coordinated to a co-ligand and a substrate containing a half spin nucleus to form a hyperpolarized substrate that is free or substantially free of the perfluorinated SABRE catalyst, in accordance with an aspect of the invention.

FIG. 6 depicts, in the top curve, a single-scan HP ¹³C spectrum obtained from the activated SABRE-SHEATH experiment of 30 mM sodium [1-¹³C]pyruvate, 42 mM p-trifluoromethyl phenyl sulfoxide (PTFSO), 7.8 mM Ir—F-IMes shown in Example 5 in deuterated methanol. The spectrum was acquired immediately following manual sample transfer to a 1.88 T benchtop NMR after 55 seconds of 50% p-H₂ bubbling at BT=−0.7 μT. The bottom curve shows a single-scan thermally polarized ¹³C signal from 4 M sodium [1-¹³C] acetate using similar acquisition parameters. Enhancement is ε˜1250 and polarization is about P(¹³C)˜0.1%.

FIG. 7 provides a graph showing the perfluorinated SABRE catalyst activation time, as described in Example 6.

FIG. 8 provides a schematic showing the formation of Complex 2, Complex 3a, Complex 3b, and pyruvate, as described in Example 6.

FIGS. 9A and 9B provide graphs showing the polarization (%) as a function of parahydrogen flow rate and pressure, respectively, as described in Example 7.

FIGS. 10A and 10B provide graphs showing the level of ¹³C polarization transfer as a function of temperature and magnetic field, respectively, as described in Example 7.

FIGS. 11A and 11B provide graphs showing the polarization (%) as a function of perfluorinated SABRE catalyst concentration and DMSO concentration, respectively, as described in Example 7.

FIGS. 12A and 12B provide graphs showing the relaxation dynamics of [1-¹³C]-pyruvate, prepared by the method set forth in Example 7, where FIG. 12A shows the buildup and relaxation at 0.4 μT and FIG. 12B shows the relaxation at 1.88 T and the Earth's field.

FIG. 13 shows a comparison of a representative spectrum of ¹³C-hyperpolarized [1-¹³C]-pyruvate with signal enhancement ε of ˜86500 fold, corresponding to P_{13C} of ˜13.48%, as evidenced by the top spectrum of FIG. 13 as compared to the bottom spectrum of FIG. 13 showing a single-scan thermally polarized ¹³C signal from 4 M sodium [1-¹³C] acetate using similar acquisition parameters, as described in Example 7.

FIG. 14 shows a variable temperature SABRE-SHEATH experiment using the saturated perfluorinated SABRE catalyst of Example 4, as described in Example 7.

FIG. 15 shows a comparison of a representative spectrum of ¹³C-hyperpolarized [1-¹³C]-pyruvate after reconstitution in water with signal enhancement ε of ˜9000 fold, corresponding to P_{13C} of ˜2.17%, as evidenced by the top spectrum of FIG. 15 as compared to the bottom spectrum of FIG. 15 showing a single-scan thermally polarized ¹³C signal from 4 M sodium [1-¹³C] acetate using similar acquisition parameters, as described in Example 9.

FIG. 16 shows a comparison of a representative spectrum of ¹³C-hyperpolarized [1-¹³C]-pyruvate with signal enhancement ε of ˜16900 fold, corresponding to P_{13C} of ˜2.17%, as evidenced by the top spectrum of FIG. 16 as compared to the bottom spectrum of FIG. 16 showing a single-scan thermally polarized ¹³C signal from 4 M sodium [1-¹³C] acetate using similar acquisition parameters, as described in Example 17.

FIG. 17 shows a comparison of a representative spectrum of ¹³C-hyperpolarized [1-¹³C]-pyruvate with signal enhancement ε of ˜19000 fold, corresponding to P_{13C} of ˜4.91%, as evidenced by the top spectrum of FIG. 17 as compared to the bottom spectrum of FIG. 17 showing a single-scan thermally polarized ¹³C signal from 4 M sodium [1-¹³C] acetate using similar acquisition parameters, as described in Example 23.

FIG. 18 provides a graph showing the perfluorinated SABRE catalyst activation time in a fluorous mixture, as described in Example 24.

FIG. 19 provides a graph showing the polarization (%) as a function of parahydrogen flow rate in a fluorous mixture, as described in Example 25.

FIG. 20 provides a graph showing the level of ¹³C polarization transfer as a function of magnetic field in a fluorous mixture, as described in Example 25.

FIGS. 21A and 21B provide graphs showing the relaxation dynamics of [1-¹³C]-pyruvate in a fluorous mixture, prepared by the method set forth in Example 25, where FIG. 21A shows the buildup and relaxation at 0.4 μT and FIG. 21B shows the relaxation at 1.88 T and the Earth's field.

FIG. 22 shows a comparison of a representative spectrum of ¹³C-hyperpolarized [1-¹³C]-pyruvate in a fluorous mixture with signal enhancement ε of ˜38600 fold, corresponding to P_{13C} of ˜6.02%, as evidenced by the top spectrum of FIG. 22 as compared to the bottom spectrum of FIG. 22 showing a single-scan thermally polarized ¹³C signal from 4 M sodium [1-¹³C] acetate using similar acquisition parameters, as described in Example 25.

FIG. 23 shows a variable temperature SABRE-SHEATH experiment using the saturated perfluorinated SABRE catalyst of Example 4 in a fluorous mixture, as described in Example 25.

FIG. 24 shows a comparison of a representative spectrum of ¹³C-hyperpolarized [1-¹³C]-pyruvate with signal enhancement ε of ˜10800 fold, corresponding to P_{13C} of ˜1.68%, as evidenced by the top spectrum of FIG. 24 as compared to the bottom spectrum of FIG. 24 showing a single-scan thermally polarized ¹³C signal from 4 M sodium [1-¹³C] acetate using similar acquisition parameters, as described in Example 26.

FIG. 25 shows a comparison of representative spectra of ¹³C-hyperpolarized [1-¹³C]-pyruvate, prepared by a re-used catalyst, with signal enhancement F of ˜3090 fold, corresponding to P_{13C} of ˜0.48%, as evidenced by the bottom two spectra of FIG. 25 as compared to the top spectrum of FIG. 25 showing a single-scan thermally polarized ¹³C signal from 4 M sodium [1-¹³C] acetate using similar acquisition parameters, as described in Example 26.

FIG. 26 shows an illustrative synthesis of 1,3-dimesityl-4,5-bis(2-(perfluorophenyl)ethyl)-4,5-dihydro-1H-imidazol-3-ium, triflate salt, as described in Example 27.

FIG. 27 provides a graph showing the perfluorinated SABRE catalyst activation time, as described in Example 31.

FIG. 28 provides a graph showing the polarization (%) as a function of parahydrogen flow rate, as described in Example 31.

FIGS. 29A and 29B provide graphs showing the level of ¹³C polarization transfer as a function of temperature and magnetic field, respectively, as described in Example 31.

FIGS. 30A and 30B provide graphs showing the relaxation dynamics of [1-¹³C]-pyruvate, prepared by the method set forth in Example 31, where FIG. 30A shows the buildup and relaxation at 0.4 μT and FIG. 30B shows the relaxation at 1.88 T and the Earth's field.

FIG. 31 provides a graph showing the polarization (%) as a function of DMSO concentration at 0° C. and room temperature, as described in Example 31.

FIG. 32 provides a comparison of a representative spectrum of ¹³C-hyperpolarized [1-¹³C]-pyruvate with signal enhancement ε of ˜104010 fold, corresponding to P_{13C} of ˜16.2%, as evidenced by the top spectrum of FIG. 32 as compared to the bottom spectrum of FIG. 32 showing a single-scan thermally polarized ¹³C signal from 4 M sodium [1-¹³C] acetate using similar acquisition parameters, as described in Example 31.

FIG. 33 provides a graph showing the perfluorinated SABRE catalyst activation time, as described in Example 32.

FIG. 34 provides a graph showing the polarization (%) as a function of parahydrogen flow rate, as described in Example 32.

FIG. 35 provides a graph showing the level of ¹³C polarization transfer as a function of temperature, as described in Example 32.

FIG. 36 provides a spectrum showing a temperature sweep, which defines the distribution among the four main species shown in FIG. 8.

FIGS. 37A and 37B provide graphs showing the relaxation dynamics of [1-¹³C]-pyruvate, prepared by the method set forth in Example 32, where FIG. 37A shows the buildup and relaxation at 0.4 μT and FIG. 37B shows the relaxation at 1.88 T and the Earth's field.

FIG. 38 provides a graph showing the polarization (%) as a function of phenyl trifluoromethyl sulfoxide (PTMSO) concentration at room temperature, as described in Example 32.

FIG. 39 shows a comparison of a representative spectrum of ¹³C-hyperpolarized [1-¹³C]-pyruvate with signal enhancement ε of ˜92018 fold, corresponding to P_{13C} of ˜14.35%, as evidenced by the top spectrum of FIG. 39 as compared to the bottom spectrum of FIG. 39 showing a single-scan thermally polarized ¹³C signal from 4 M sodium [1-¹³C] acetate using similar acquisition parameters, as described in Example 32.

FIG. 40 depicts an exemplary purification process protocol consisting of buffered-heavy water (D₂O) addition, solvent evaporation under vacuum in a hot water bath, followed by solution filtering, and finally ¹³C NMR signal detection by 80 MHz spectrometer, as utilized in Examples 33 and 34.

FIG. 41 provides ¹³C NMR spectra of [1-¹³C]pyruvate after SABRE hyperpolarization using DMSO as a co-ligand in fluorinated solvent before the purification process (top) and after the purification process protocol (middle) compared with an external ¹³C-enriched reference solution (bottom) at 1.88 T, as described in Example 33.

FIG. 42 provides ¹³C NMR spectra of [1-¹³C]pyruvate after SABRE hyperpolarization using PTMSO as a co-ligand in fluorinated solvent before the purification process (top) and after the purification process protocol (middle) compared with an external ¹³C-enriched reference solution (bottom) at 1.88 T, as described in Example 34.

FIG. 43 provides a schematic for an exemplary perfluorinated SABRE-SHEATH setup, followed by biphasic [1-¹³C]pyruvate extraction from the perfluorinated SABRE catalyst (a)-(d), as utilized in Example 35. (a) A 5 mm NMR tube is pressurized with p-H₂ (˜8 bar total pressure regulated by a safety valve) flowing through the sample at a fixed flow rate of 90 scc/m (standard cubic centimeters per minute) using a digital mass flow controller (MFC). Hyperpolarization occurred within a mu-metal shielded electromagnet, with the magnetic field adjusted to approximately B_{transfer}≈0.4 μT and T_{transfer} set at either 0° C. or room temperature. (b) After 30 sec of p-H₂ bubbling the sample is transferred to the spectrometer B=1.88 T to minimize T₁ losses. (c) Sample is depressurized and 300 μL of water is gently added at the bottom of the sample, which allows the extraction of [1-¹³C]pyruvate from the fluorinated phase into the aqueous phase. (d) By adjusting the 9-inch NMR tube deeper into the spectrometer, the water phase is positioned within the spectrometer's sensing zone, enabling the measurement of the ¹³C signal enhancement of [1-¹³C]pyruvate.

FIG. 44A provides ¹³C NMR spectra of [1-¹³C]pyruvate after SABRE hyperpolarization using DMSO as a co-ligand in fluorinated solvent before the purification process (top) and after the purification process protocol (middle) compared with an external ¹³C-enriched reference solution (bottom) at 1.88 T, as described in Example 35.

FIG. 44B provides ¹³C NMR spectra of [1-¹³C]pyruvate after SABRE hyperpolarization using PTMSO as a co-ligand in fluorinated solvent before the purification process (top) and after the purification process protocol (middle) compared with an external ¹³C-enriched reference solution (bottom) at 1.88 T, as described in Example 35.

FIG. 45 provides a schematic for an exemplary perfluorinated SABRE-SHEATH setup, followed by biphasic [1-¹³C]pyruvate extraction and recycle and reuse of the perfluorinated SABRE catalyst (a)-(d), as utilized in Example 36. (a) A 5 mm NMR tube is pressurized with p-H₂ (˜8 bar total pressure regulated by a safety valve) flowing through the sample at a fixed flow rate of 90 scc/m (standard cubic centimeters per minute) using a digital mass flow controller (MFC). Hyperpolarization occurred within a mu-metal shielded electromagnet, with the magnetic field adjusted to approximately B_{transfer}≈0.4 μT and T_{transfer} set at either 0° C. or room temperature. (b) After 30 sec of p-H₂ bubbling the sample is transferred to the spectrometer B=1.88 T to minimize T₁ losses, the sample is depressurized, and 300 μL of water is added to the sample, which allows the extraction of [1-¹³C]pyruvate from the fluorinated phase. (c) The next step for the recovery and reuse of the perfluorinated catalyst includes evacuating the aqueous phase which contains the pyruvate and adding to the fluorinated phase a newly prepared methanol solution containing 30 mM [1-¹³C]pyruvate and co-ligand. The samples are hyperpolarized as describe in step (a). (d) Finally, the last step is a repetition of step (b) and includes a second [1-¹³C]pyruvate extraction with 300 μL of water.

FIG. 46A provides ¹³C NMR spectra of 30 mM [1-¹³C]pyruvate after SABRE hyperpolarization using DMSO as a co-ligand in fluorinated solvent (top), ¹³C NMR spectra of the same solution after the extraction process with heavy water (D₂O) (second from top), ¹³C NMR spectra of 30 mM [1-¹³C]pyruvate after recycling the catalysis (middle), and ¹³C NMR spectra of the same solution after the second extraction process with heavy water (D₂O) (second from bottom), compared with an external ¹³C-enriched reference solution (black) at 0.81 T, as described in Example 36.

FIG. 46B provides ¹³C NMR spectra of 30 mM [1-¹³C]pyruvate after SABRE hyperpolarization using PTMSO as a co-ligand in fluorinated solvent (top), ¹³C NMR spectra of the same solution after the extraction process with heavy water (D₂O) (second from top), and ¹³C NMR spectra of 30 mM [1-¹³C]pyruvate after recycling the catalysis (second from bottom), compared with an external ¹³C-enriched reference solution (black) at 0.81 T, as described in Example 36.

### DETAILED DESCRIPTION OF THE INVENTION

The present invention provides a perfluorinated SABRE catalyst comprising a d-block element and a perfluorinated ligand, wherein the perfluorinated ligand is of Formula (I):

[Lₘ-(NHC)—(Y—Z)_{q}] Formula (I),
or a salt thereof, and wherein
each L is independently selected from hydrogen, adamantyl, a substituted or unsubstituted aromatic group, or a substituted or unsubstituted heteroaromatic group,
NHC is a 4 to 7-membered N-heterocyclic carbenyl group where NHC is bound to the d-block element via a carbene,
each Y is independently selected from a bond or a spacer group,
each Z is a perfluorinated tag,
m is an integer from 1 to 4 (e.g., 1, 2, 3, or 4), and
q is an integer from 1 to 3 (e.g., 1, 2, or 3).

The perfluorinated SABRE catalyst comprises a d-block element such as, for example, Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, and/or Hg. In some embodiments, the d-block element is a transition metal such as, for example, Co, Rh, Ir, Ru, Pd, Pt, or Mt. In certain embodiments, the perfluorinated SABRE catalyst comprises an element of group 9 of the periodic table, i.e., Co, Rh, Ir, or Mt. In preferred embodiments, the perfluorinated SABRE catalyst comprises Ir or Co. For example, the perfluorinated SABRE catalyst can be prepared from [Ir(COD)(IMes)(Cl)].

The perfluorinated SABRE catalyst comprises a perfluorinated ligand of Formula (I):

[Lₘ-(NHC)—(Y—Z)_{q} Formula (I),
or a salt thereof, and wherein
   each L is independently selected from hydrogen, adamantyl, a substituted or unsubstituted aromatic group, or a substituted or unsubstituted heteroaromatic group,
   NHC is a 4 to 7-membered N-heterocyclic carbenyl group where NHC is bound to the d-block element via a carbene,
   each Y is independently selected from a bond or a spacer group,
   each Z is a perfluorinated tag,
   m is an integer from 1 to 4 (e.g., 1, 2, 3, or 4), and
   q is an integer from 1 to 3 (e.g., 1, 2, or 3).

In some aspects, NHC comprises an azolyl moiety, i.e., a five membered heterocyclic group having a nitrogen atom and at least one other hetero atom selected from nitrogen, sulfur, and oxygen. Thus, in some embodiments, NHC is a 5-membered N-heterocyclic carbenyl group. For example, the 5-membered N-heterocyclic carbenyl group can be imidazole-based, imidazoline-based, or thiazole-based. In other words, the 5-membered N-heterocyclic carbenyl group can be the resulting carbene formed from treatment of a perfluorinated ligand having an imidazole, an imidazoline, or a thiazole core.

In some embodiments, NHC is a 4,5-disubstituted, a 1,3-disubstituted, or a 1,3,4,5-tetrasubstituted imidazole-based or imidazoline-based 5-membered N-heterocyclic carbenyl group. For example, NHC can be a 4,5-disubstituted imidazolidinyl, a 1,3-disubstituted imidazolidinyl, a 1,3,4,5-tetrasubstituted imidazolidinyl, a 4,5-disubstituted 2,3-dihydro-imidazolyl, a 1,3-disubstituted 2,3-dihydro-imidazolyl, or a 1,3,4,5-tetrasubstituted 2,3-dihydro-imidazolyl. Examples of the imidazolylidinyl moiety include N,N′-di-(2,4,6-trimethylphenyl)-imidazolylidinyl moiety, N,N′-di-(2,6-diisopropylphenyl)-imidazolidinyl moiety, N,N′-di-(2,6-dicyclohexyl)-imidazolidinyl moiety, N,N′-di-(2,6-t-butyl)-imidazolidinyl moiety, and N,N′-di-(1-adamantyl)-imidazolidinyl moiety.

In some embodiments, the perfluorinated ligand is of Formula (Ia) or (Ib):

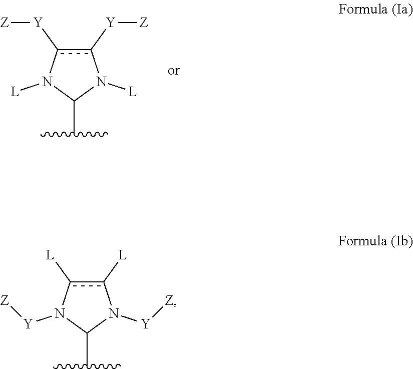

or a salt thereof, and wherein
   each L independently is hydrogen, adamantyl, a substituted or unsubstituted aromatic group, or a substituted or unsubstituted heteroaromatic group,
   each Y independently is a bond or a spacer group,
   [symbol] each Z independently is a perfluorinated tag, is a single bond or a double bond, and
   [symbol] represents the bond to the d-block element via the carbene.

In some embodiments, the perfluorinated ligand is of Formula (Ic) or (Id):

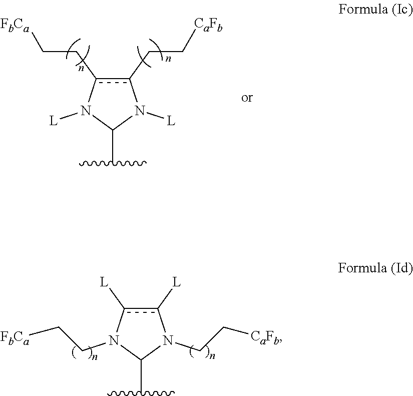

or a salt thereof, and wherein
   each L independently is hydrogen, adamantyl, a substituted or unsubstituted aromatic group, or a substituted or unsubstituted heteroaromatic group,
   a is 4 to 20 (e.g., 4, 5, 6, 7, 8, 9, 10, 11, 12, 13, 14, 15, 16, 17, 18, 19, or 20),
   b=2a+1 or b=a−1,
   each n independently is an integer from 0 to 4 (e.g., 0, 1, 2, 3, or 4),
   [symbol] is a single bond or a double bond, and
   [symbol] represents the bond to the d-block element via the carbene. In some embodiments of Formula (Ic) or (Id) a is 4 to 10 (e.g., 4, 5, 6, 7, 8, 9, or 10).

In some embodiments, the perfluorinated ligand is of Formula (Ie) or (If):

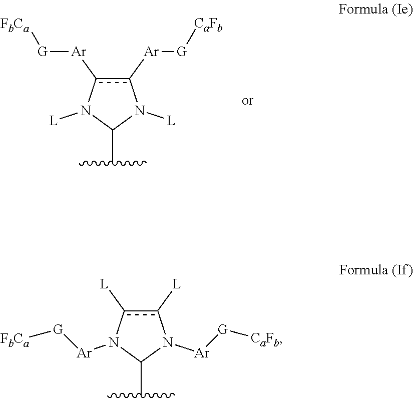

or a salt thereof, and wherein
   each L independently is hydrogen, adamantyl, a substituted or unsubstituted aromatic group, or a substituted or unsubstituted heteroaromatic group,
   each Ar independently is a substituted or unsubstituted aromatic group, or a substituted or unsubstituted heteroaromatic group,
   each G independently is a bond, C₁-6 alkyl, C₁-6 alkenyl, or C₁-6 heteroalkyl,
   a is 4 to 20 (e.g., 4, 5, 6, 7, 8, 9, 10, 11, 12, 13, 14, 15, 16, 17, 18, 19, or 20),
   b=2a+1 or b=a−1,
   [symbol] is a single bond or a double bond, and
   [symbol] represents the bond to the d-block element via the carbene. In some embodiments of Formula (Ie) or (If), a is 4 to 10 (e.g., 4, 5, 6, 7, 8, 9, or 10).

In any of Formulae (I) and (Ia)-(If), each L is independently selected from hydrogen, adamantyl, a substituted or unsubstituted aromatic group, or a substituted or unsubstituted heteroaromatic group.

As used herein, “substituted or unsubstituted aromatic” refers to a substituted (e.g., C₁-6 alkyl substituted) or unsubstituted aromatic ring having 5 to 60 ring carbon atoms, e.g., phenyl, naphthyl, phenanthryl, and anthracenyl. As used herein, “substituted or unsubstituted heteroaromatic” refers to a substituted (e.g., C₁-6 alkyl substituted) or unsubstituted aromatic ring having from 1 to 2 heteroatoms chosen from N, O, and S, with remaining ring atoms being carbon, or a stable bicyclic or tricyclic system containing at least one 5- to 7-membered aromatic ring which contains from 1 to 3, or in some aspects, from 1 to 2, heteroatoms chosen from N, O, and S, with remaining ring atoms being carbon. Monocyclic heteroaryl groups typically have from 5 to 7 ring atoms, in some aspects, bicyclic heteroaryl groups are 9- to 10-membered heteroaryl groups, that is, groups containing 9 or 10 ring atoms in which one 5- to 7-member aromatic ring is fused to a second aromatic or non-aromatic ring. When the total number of S and O atoms in the heteroaryl group exceeds 1, these heteroatoms are not adjacent to one another. It is preferred that the total number of S and O atoms in the heteroaryl group is not more than 2. It is particularly preferred that the total number of S and O atoms in the aromatic heterocycle is not more than 1. Heteroaromatic groups include, but are not limited to, oxazolyl, piperazinyl, pyranyl, pyrazinyl, pyrazolopyrimidinyl, pyrazolyl, pyridizinyl, pyridyl, pyrimidinyl, pyrrolyl, quinolinyl, tetrazolyl, thiazolyl, thienylpyrazolyl, thiophenyl, triazolyl, benzoisoxazolyl, benzofuranyl, benzothiazolyl, benzothiophenyl, benzoxadiazolyl, dihydrobenzodioxynyl, furanyl, imidazolyl, indolyl, isothiazolyl, and isoxazolyl.

In some embodiments, each L independently is hydrogen, adamantyl, 2-methylphenyl, 3-methylphenyl, 4-methylphenyl, 2,4-dimethylphenyl, 2,5-dimethylphenyl, 2,6-dimethylphenyl, 3,5-dimethylphenyl, 2,4,6-trimethylphenyl, 2-ethylphenyl, 3-ethylphenyl, 4-ethylphenyl, 2,4-diethylphenyl, 2,5-diethylphenyl, 2,6-diethylphenyl, 3,5-diethylphenyl, 2,4,6-triethylphenyl, 2-npropylphenyl, 3-npropylphenyl, 4-npropylphenyl, 2,4-di-npropylphenyl, 2,5-di-npropylphenyl, 2,6-di-npropylphenyl, 3,5-di-npropylphenyl, 2,4,6-tri-npropylphenyl, 2-isopropylphenyl, 3-isopropylphenyl, 4-isopropylphenyl, 2,4-di-isopropylphenyl, 2,5-di-isopropylphenyl, 2,6-di-isopropylphenyl, 3,5-di-isopropylphenyl, 2,4,6-tri-isopropylphenyl, 2-isobutylphenyl, 3-isobutylphenyl, 4-isobutylphenyl, 2,4-di-isobutylphenyl, 2,5-di-isobutylphenyl, 2,6-di-isobutylphenyl, 3,5-di-isobutylphenyl, 2,4,6-tri-isobutylphenyl, 2-secbutylphenyl, 3-secbutylphenyl, 4-secbutylphenyl, 2,4-di-secbutylphenyl, 2,5-di-secbutylphenyl, 2,6-di-secbutylphenyl, 3,5-di-secbutylphenyl, 2,4,6-tri-secbutylphenyl, 2-tbutylphenyl, 3-tbutylphenyl, 4-tbutylphenyl, 2,4-di-tbutylphenyl, 2,5-di-tbutylphenyl, 2,6-di-tbutylphenyl, 3,5-di-tbutylphenyl, 2,4,6-tri-tbutylphenyl, 2-cyclohexylphenyl, 3-cyclohexylphenyl, 4-cyclohexylphenyl, 2,4-di-cyclohexylphenyl, 2,5-di-cyclohexylphenyl, 2,6-di-cyclohexylphenyl, 3,5-di-cyclohexylphenyl, or 2,4,6-tri-cyclohexylphenyl. In certain embodiments of Formulae (I) and (Ia)-(If), each L independently is hydrogen or 2,4,6-trimethylphenyl.

In any of Formulae (I) and (Ia)-(If), each Y independently is a bond or a spacer group. For example, Y can be a bond, a substituted or unsubstituted C₁₋₁₀ alkyl group, a substituted or unsubstituted C₂₋₁₀ alkenyl group, a substituted or unsubstituted C₂₋₁₀ alkynyl group, a substituted or unsubstituted C₁₋₁₀ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, or a linear or branched alkyleneoxy group (e.g., polyethylene oxide, polypropylene oxide, or a combination thereof).

In some embodiments, each Y independently is a bond, a substituted or unsubstituted C₁₋₁₀ alkyl group, a substituted or unsubstituted C₂₋₁₀ alkenyl group, a substituted or unsubstituted C₂₋₁₀ alkynyl group, a substituted or unsubstituted C₁₋₁₀ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, or a linear or branched alkyleneoxy group. In certain embodiments, each Y independently is a bond, a substituted or unsubstituted C₁₋₁₀ alkyl group, a substituted or unsubstituted C₂₋₁₀ alkenyl group, a substituted or unsubstituted aryl group, a substituted or unsubstituted heteroaryl group, a substituted or unsubstituted alkaryl group, or a substituted or unsubstituted arylalkyl group. In preferred embodiments, each Y independently is a bond, a substituted or unsubstituted C₁₋₁₀ alkyl group, a substituted or unsubstituted C₂₋₁₀ alkenyl group, a substituted or unsubstituted aryl group, a substituted or unsubstituted heteroaryl group, a substituted or unsubstituted alkaryl group, or a substituted or unsubstituted arylalkyl group.

The perfluorinated ligand comprises a perfluorinated tag. For example, in any of Formulae (I) and (Ia)-(If), each Z is a perfluorinated tag. The perfluorinated tag can be any perfluorinated group such as, for example, a perfluorinated alkyl (e.g., linear or branched), aryl, alkyarl, or arylalkyl group containing up to 60 carbon atoms. In some embodiments, the perfluorinated tag is a perfluorinated C₃₋₆₀ group comprising only carbon and fluorine atoms. In certain embodiments, the perfluorinated tag is a perfluorinated C₃₋₄₀ group comprising only carbon and fluorine atoms. In other embodiments, the perfluorinated tag is a perfluorinated C₃₋₂₀ group. For example, the perfluorinated tag can be selected from a C₄F₉ group, a C₅F₁₁ group, a C₆F₁₃ group, a C₇F₁₅ group, a C₈F₁₇ group, a C₉F₁₉ group, a C₁₀F₂₁ group, a C₆F₅ group, C₄F₇ group, a C₅F₉ group, a C₆F₁₁ group, a C₇F₁₃ group, a C₈F₁₅ group, a C₉F₁₇ group, and a C₁₀F₁₉ group, each of which can be a linear or branch alkyl, aryl, alkyarl, or arylalkyl group.

In an aspect, Z is a perfluoroalkyl chain, linear or branched, having a chain length of up to 60 or more carbon atoms, for example, the perfluoroalkyl chain has a chain length of 3-60, particularly, 3 to 40, more particularly 3 to 20, and even more particularly 3 to 10 or more, carbon atoms. For example, the perfluoroalkyl chain is selected from the group consisting of C₄F₉, C₆F₁₃, C₇F₁₅, C₈F₁₇, C₉F₁₉, and C₁₀F₂₁, preferably selected from the group consisting of C₆F₁₃, C₈F₁₇, and C₁₀F₂₁, each of which can be linear or branched and combinations thereof, wherein each of which can be linear or branched.

In an aspect, the perfluorinated ligand is of formula (Ig):

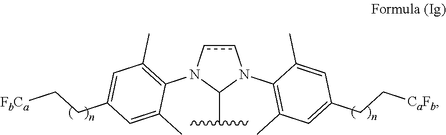

or a salt thereof, and wherein
   a is 4 to 20 (e.g., 4, 5, 6, 7, 8, 9, 10, 11, 12, 13, 14, 15, 16, 17, 18, 19, or 20),
   b=2a+1 or b=a−1,
   [symbol] is a single bond or a double bond, and
   [symbol] represents the bond to the d-block element via the carbene. In some embodiments of Formula (Ig), a is 4 to 10 (e.g., 4, 5, 6, 7, 8, 9, or 10).

In an aspect, the perfluorinated ligand is of formula (Ih):

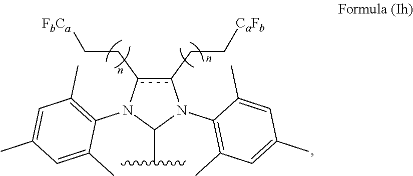

or a salt thereof, and wherein
   a is 4 to 20 (e.g., 4, 5, 6, 7, 8, 9, 10, 11, 12, 13, 14, 15, 16, 17, 18, 19, or 20),
   b=2a+1 or b=a−1,
   [symbol] is a single bond or a double bond, and
   [symbol] represents the bond to the d-block element via the carbene. In some embodiments of Formula (Ig), a is 4 to 10 (e.g., 4, 5, 6, 7, 8, 9, or 10).

Exemplary perfluorinated ligands include:

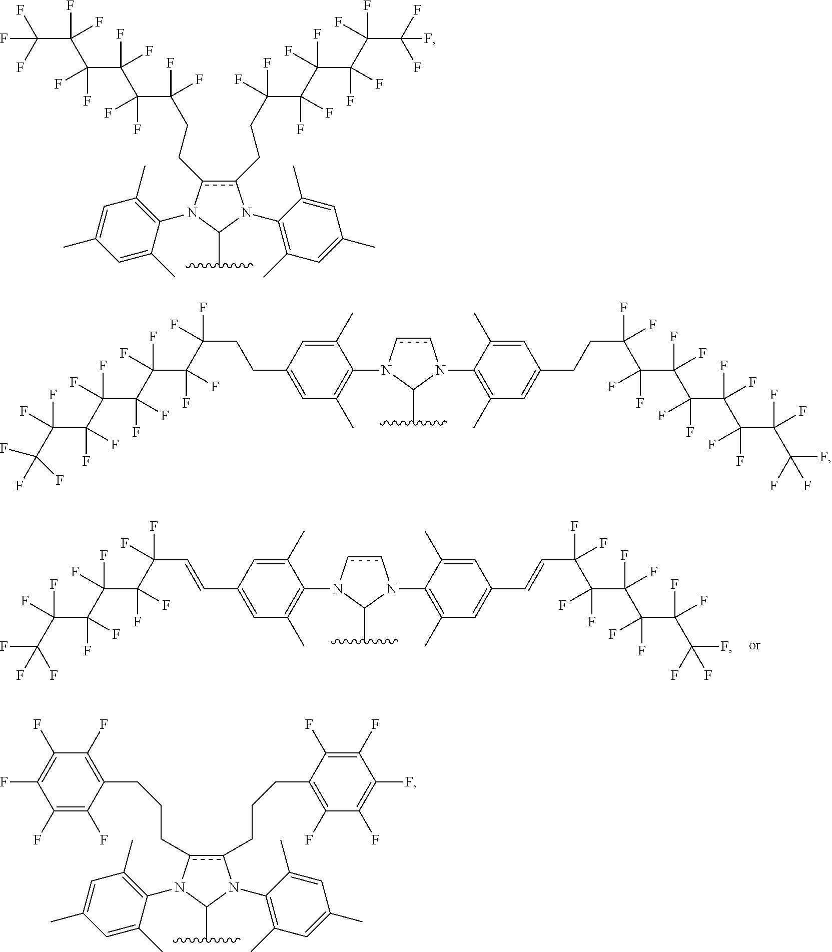

Text use or a salt thereof, and wherein [symbol] is a single bond or a double bond, and [symbol] represents the bond to the d-block element via the carbene.

As used herein, the symbol “ [symbol] ” represents a single bond or a double bond. In some embodiments, [symbol] is a single bond. In other embodiments, [symbol] is a double bond. In embodiments where [symbol] is a single bond, the orientation of the two substituents stemming from [symbol] can have any suitable stereochemistry, i.e., can be cis or trans. In preferred embodiments, when [symbol] is a single bond, the stereochemistry of the substituents stemming from [symbol] is trans.

As used herein, the symbol “ [symbol] ”represents the bond to the d-block element via the carbene. In other words, [symbol] represents the bond to the metal of the catalyst.

In some embodiments, the perfluorinated SABRE catalyst further comprises an additional ligand. For example, the perfluorinated SABRE catalyst may further comprise an additional ligand selected from phosphine ligands, carbene ligands, imidazole ligands, pincer chelating ligands, and compounds comprising a sulfoxide group. In certain embodiments, the perfluorinated SABRE catalyst comprises one or more phosphine ligands. Examples of phosphine ligands include, but are not limited to the following:

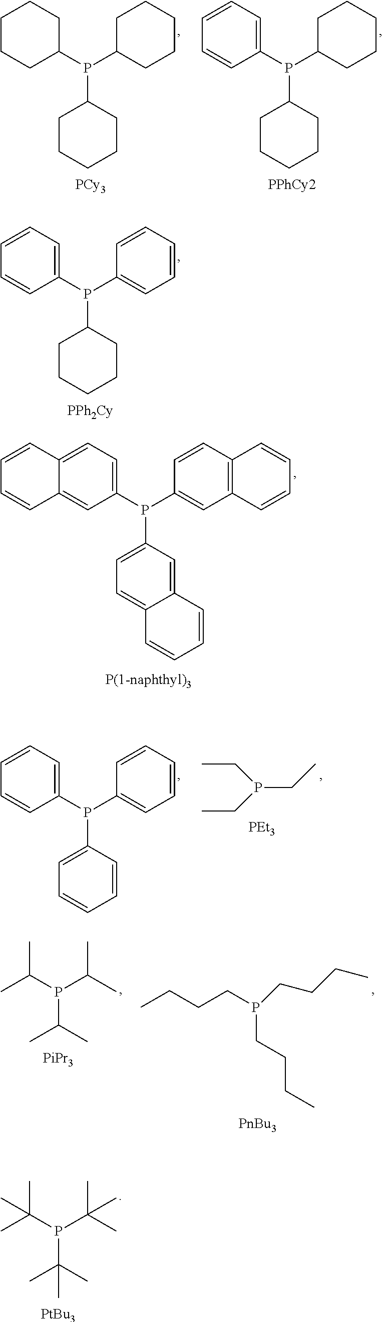

In some embodiments, the perfluorinated SABRE catalyst comprises a pincer chelating ligand. Generally, when the perfluorinated SABRE catalyst comprises a phosphine ligand or a pincer chelating ligand, the perfluorinated SABRE catalyst is in pre-catalyst form. In some embodiments, the perfluorinated SABRE catalyst comprises a ligand that is a compound comprising a sulfoxide group. Examples of compounds comprising a sulfoxide group can be selected from the group consisting of dimethylsulfoxide (DMSO), phenyl trifluoromethyl sulfoxide, phenyl methyl sulfoxide, phenyl chloromethyl sulfoxide, diphenyl sulfoxide, dibenzoyl sulfoxide, 2,2,2-trifluoroethyl methane sulfonate, di-2,2,2-trifluoroethyl sulfoxide, and dibutyl sulfoxide. Generally, when the perfluorinated SABRE catalyst comprises a compound comprising a sulfoxide group, the perfluorinated SABRE catalyst is in active form. As used herein, “the perfluorinated SABRE catalyst” can refer to the active perfluorinated SABRE catalyst or the perfluorinated SABRE precatalyst.

The active perfluorinated SABRE catalyst can be prepared by any suitable method. Generally, the active perfluorinated SABRE catalyst is prepared by combining the perfluorinated SABRE precatalyst with a substrate, parahydrogen, and optionally a co-ligand in a solvent to form a mixture comprising an active perfluorinated SABRE catalyst. In some embodiments, the active perfluorinated SABRE catalyst is prepared by combining the perfluorinated SABRE precatalyst with a substrate, parahydrogen, and a co-ligand in a solvent to form a mixture comprising an active perfluorinated SABRE catalyst. The co-ligand, when included in the preparation of the active perfluorinated SABRE catalyst, can be combined with the perfluorinated SABRE precatalyst in any order and by any suitable means. For example, the co-ligand, when included in the preparation of the active SABRE catalyst, can be provided first to interact with the transfer precatalyst to facilitate formation of the active perfluorinated SABRE catalyst. Alternatively, the co-ligand, when included in the preparation of the active perfluorinated SABRE catalyst, can be added together with the substrate to facilitate formation of the active perfluorinated SABRE catalyst. In some embodiments, the co-ligand, the substrate, and parahydrogen are essentially combined with the perfluorinated SABRE precatalyst in the solvent at the same time to facilitate formation of the active perfluorinated SABRE catalyst. In other embodiments, the substrate is provided first to interact with the perfluorinated SABRE precatalyst to facilitate formation of the active SABRE catalyst. In some embodiments, the co-ligand and the substrate are combined with the perfluorinated SABRE precatalyst in the solvent, and the parahydrogen is added to (e.g., bubbled through) the resulting mixture. In other embodiments, the substrate is combined with the perfluorinated SABRE precatalyst in the solvent, and the parahydrogen is added to (e.g., bubbled through) the resulting mixture. In some embodiments, the active perfluorinated SABRE catalyst is prepared by combining the perfluorinated SABRE precatalyst with a co-ligand in addition to the substrate and parahydrogen.

In some embodiments, the active perfluorinated SABRE catalyst is of formula

[Ir(H)₂(F-IMes)(η²-SUBSTRATE)(Co-ligand)] or

[Ir(H)₂(F-IMes)(η¹-SUBSTRATE)(Co-ligand)₂],

wherein SUBSTRATE is a target substrate to be hyperpolarized by the transfer of the pure singlet spin order of parahydrogen by the spin transfer catalyst, preferably a target substrate having enriched with an atom having 12 spin nuclei, for example, ¹H, ¹³C, ¹⁵N, ¹⁹F, ³¹P and/or ²⁹Si. Without wishing to be bound by any particular theory, the co-ligand interacts with the spin transfer pre-catalyst to form the activated polarization transfer catalyst and enhances the polarization transfer to the target substrate. F-IMes refers to a perfluorinated form of N-heterocyclic carbenyl (NHC) ligand such as a 1,3-Bis(2,4,6-trimethylphenyl)-1,3-dihydro-2H-imidazol-2-ylidene group or a 1,3-Bis(2,4,6-trimethylphenyl)-1,3-dihydro-4H-imidazol-2-ylidene group described herein.

In some embodiments, the perfluorinated SABRE catalyst (e.g., the perfluorinated SABRE precatalyst) can be prepared by a method comprising reacting a perfluorinated compound with a base to form a carbene, and reacting the carbene with [(d-block element)(COD)Cl]₂, wherein COD stands for 1,4-cyclooctadienyl. In certain embodiments, the method comprises reacting a perfluorinated compound with a base to form a carbene, and reacting the carbene with [Ir(COD)C]₂. For example, a perfluorinated SABRE catalyst of formula:

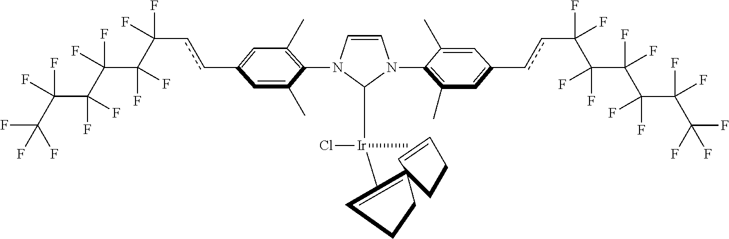

can be prepared by reacting a carbene of formula:

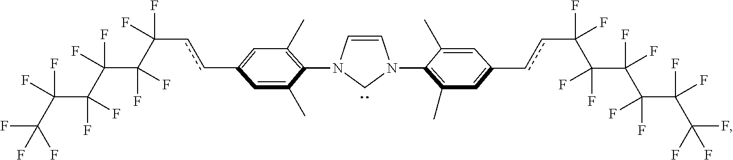

with [Ir(COD)Cl]₂.

Exemplary perfluorinated SABRE catalysts (e.g., the perfluorinated SABRE precatalyst) include:

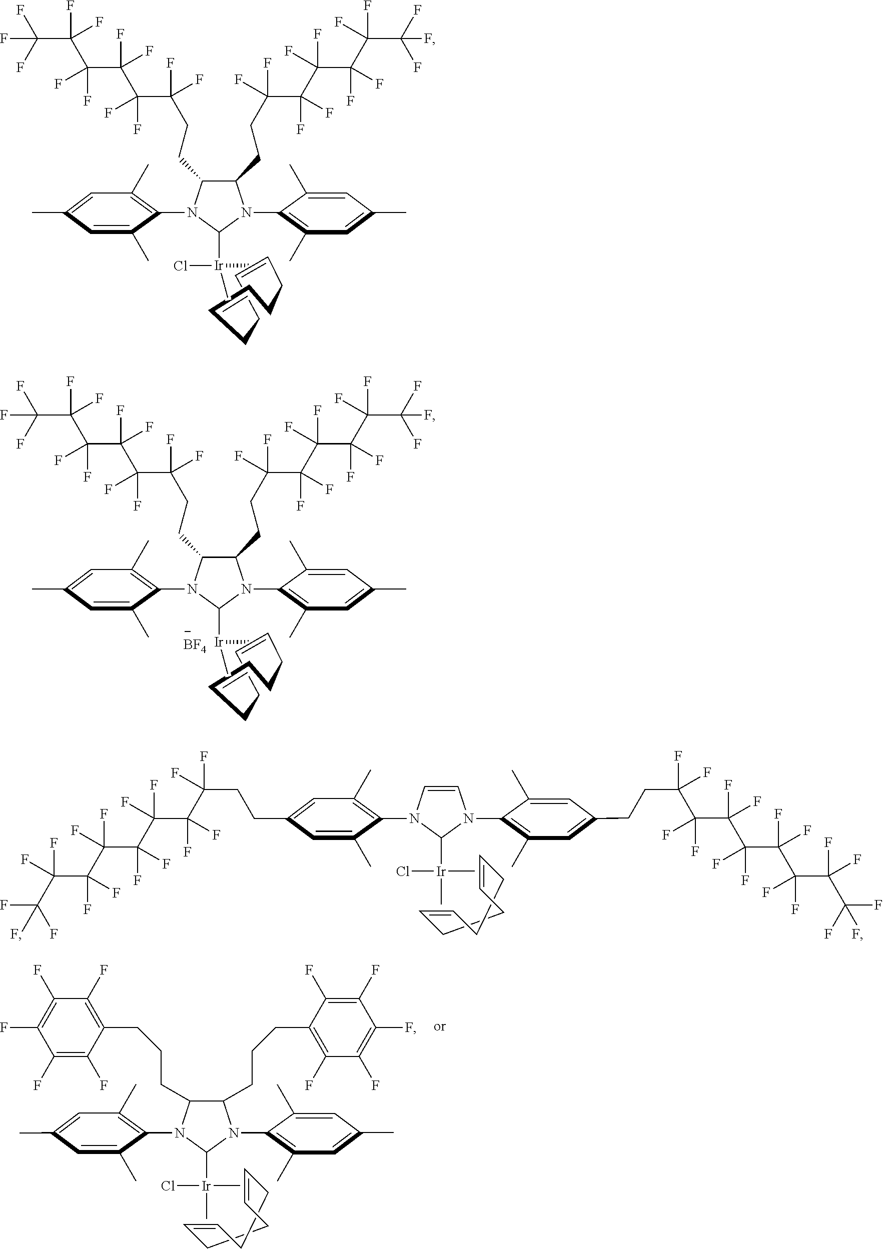

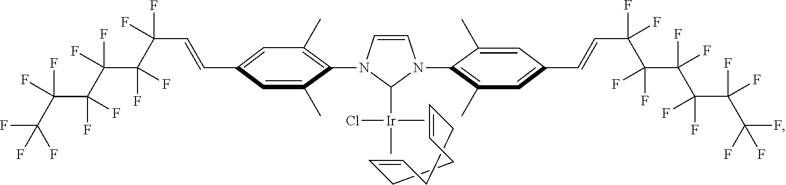

or salts thereof. Any structure (e.g., catalyst) depicted herein can exist as the depicted enantiomer, the enantiomer of the depicted compound, or a racemic mixture thereof. Generally, the stereochemistry is depicted herein to show cis-trans relationships only. As a result, any structure (e.g., catalyst) provided herein can be a single enantiomer, a mixture of enantiomers, or a racemic mixture. In some embodiments, the catalysts described herein are racemic mixtures.

The present invention further provides a method of preparing a hyperpolarized substrate, the method comprising:
(i) providing a perfluorinated SABRE catalyst described herein;
(ii) providing a co-ligand to interact with the perfluorinated SABRE catalyst to facilitate formation of an active perfluorinated SABRE catalyst;
(iii) combining the active perfluorinated SABRE catalyst with parahydrogen and a substrate comprising a ½ spin nucleus or nuclei in a solvent to obtain a reaction mixture; and
(iv) hyperpolarizing the mixture obtained in (iii) by exposing the mixture to a magnetic field or by radiofrequency excitation to obtain a hyperpolarized active perfluorinated SABRE catalyst-substrate and/or a hyperpolarized substrate.

The methods of preparing a hyperpolarized substrate, described herein, comprise hyperpolarizing the mixture comprising the perfluorinated SABRE catalyst (e.g., the active perfluorinated SABRE catalyst) by exposing the mixture to a magnetic field or radiofrequency excitation to transfer the polarization from parahydrogen to the substrate to form the hyperpolarized substrate. Initially, the hyperpolarized substrate is complexed with the hyperpolarized perfluorinated SABRE catalyst; however, it will be understood by a person of ordinary skill in the art that the hyperpolarized substrate can be replaced by another substrate molecule such that the process can be repeated and the free hyperpolarized substrate bolus is produced.

The transfer of polarization from parahydrogen to the substrate to form the hyperpolarized substrate can occur under any suitable magnetic field or radiofrequency excitation. For example, the transfer of polarization from parahydrogen to the substrate can occur at a magnetic field below the magnetic field of earth. The suitable level of magnetic field or radiofrequency excitation necessary to transfer the polarization from parahydrogen to the substrate to form the hyperpolarized substrate will be readily apparent to a person of ordinary skill in the art.

In some embodiments, the method comprises replenishing the parahydrogen in the mixture during the step of hyperpolarizing the mixture comprising the perfluorinated SABRE catalyst (e.g., the active perfluorinated SABRE catalyst) by exposing the mixture to a magnetic field or radiofrequency excitation to transfer the polarization from parahydrogen to the substrate to form the hyperpolarized substrate. In other words, in some embodiments, the method comprises bubbling parahydrogen through the mixture comprising the perfluorinated SABRE catalyst (e.g., the active perfluorinated SABRE catalyst) during the step of hyperpolarizing the mixture comprising the perfluorinated SABRE catalyst (e.g., the active perfluorinated SABRE catalyst) by exposing the mixture to a magnetic field or radiofrequency excitation to transfer the polarization from parahydrogen to the substrate to form the hyperpolarized substrate.

The method of preparing a hyperpolarized substrate comprises providing a co-ligand to interact with the perfluorinated SABRE catalyst to facilitate formation of an active perfluorinated SABRE catalyst. The co-ligand can be any suitable compound containing one or more sulfoxide groups, thioester groups, phosphine groups, amine groups, CO groups, isonitrile groups, nitrogen-containing heterocyclic groups, or a combination thereof. In some embodiments, the co-ligand is a compound comprising a sulfoxide group. Examples of compounds comprising a sulfoxide group can be selected from the group consisting of DMSO, phenyl methyl sulfoxide, phenyl chloromethyl sulfoxide, diphenyl sulfoxide, dibenzoyl sulfoxide, phenyl trifluoromethyl sulfoxide, 2,2,2-trifluoroethyl methane sulfoxide, di-2,2,2-trifluoroethyl sulfoxide, and dibutyl sulfoxide. In certain embodiments, the co-ligand is dimethyl sulfoxide. In some embodiments, the co-ligand is fluorinated. For example, the co-ligand can be phenyl trifluoromethyl sulfoxide, 2,2,2-trifluoroethyl methane sulfoxide, or di-2,2,2-trifluoroethyl sulfoxide. Without wishing to be bound by any particular theory, it is believed that a fluorinated co-ligand may help facilitate separation of the hyperpolarized substrate and the perfluorinated SABRE catalyst when performing an aqueous phase extraction and/or a fluorinated phase extraction.

In embodiments of the method of preparing a hyperpolarized substrate, the magnetic field is an electro-magnetic field. For example, the strength of the electro-magnetic field can be in the range of 0-200 milliTeslas (mT). In some embodiments, the electro-magnetic field may be at least partially supplied by one or more permanent magnets in addition to or in lieu of the coil. In some embodiments, the electro-magnetic field may be an alternating magnetic field supplied at a frequency adapted to a particular nuclei. The alternating magnetic field can change directions (i.e., alternate between positive and negative relative to a positive direction). In some embodiments, the frequency can be a radio frequency, and preferably between 50 to 500 MHz, although other frequencies outside this range are contemplated as within the scope of the present disclosure.

The perfluorinated SABRE catalyst (e.g., active perfluorinated SABRE catalyst) is combined with parahydrogen and a substrate comprising a ½ spin nucleus or nuclei in a solvent to obtain a reaction mixture. The solvent can be any suitable solvent capable of forming a heterogeneous or homogeneous mixture. In some embodiments, the solvent comprises water, methanol, ethanol, a fluorous solvent, or a mixture thereof. For example, the solvent can be ethanolic or methanolic, i.e., comprising at least ethanol or methanol in combination with water. In certain embodiments, the solvent comprises a fluorous solvent. In some embodiments, the solvent is deuterated such that a deuterated solvent can be prepared without (i.e., with limited) deuterium-hydrogen exchange.

The fluorous solvent can be any organic solvent comprising at least one compound having a fluorine atom. Without wishing to be bound by any particular theory, it is believed that the fluorous solvent increases the solubility of the perfluorinated SABRE catalyst. In some embodiments, the solvent comprises a perfluoroalkane, diethyl ether, a nonafluorobutyl methyl ether, ethyl acetate, perfluorobutyl methyl ether, a fluorocarbon derivative of THF FC 75, a decafluoromethoxy trifluoromethyl pentane, a hexafluoro propanol, a perfluoromethyl cyclohexane, and a combination thereof. In some embodiments, the solvent (e.g., the fluorous solvent) is selected from a perfluorohexane/diethyl ether mixture, a methoxy nonafluorobutane and ethyl acetate mixture with a non-polar solvent, a perfluorohexane and ether mixture, a perfluorobutyl methyl ether and ethyl acetate mixture, an ether, a fluorocarbon derivative of THF FC 75, a decafluoromethoxy trifluoromethyl pentane, a hexafluoro propanol, a nonafluorobutyl methyl ether, a perfluoromethyl cyclohexane, a perfluoroalkane, a perfluorohexane, and a methoxy nonafluorobutane. In certain embodiments, the solvent comprises water, methanol, ethanol, a nonafluorobutyl methyl ether, deuterated variants thereof, or a mixture thereof.

In some embodiments, the method of preparing a hyperpolarized substrate further comprises isolating the hyperpolarized substrate. The hyperpolarized substrate can be isolated by any suitable method. For example, the hyperpolarized substrate can be isolated by extraction, filtration, column chromatography, distillation, crystallization, or a combination thereof.

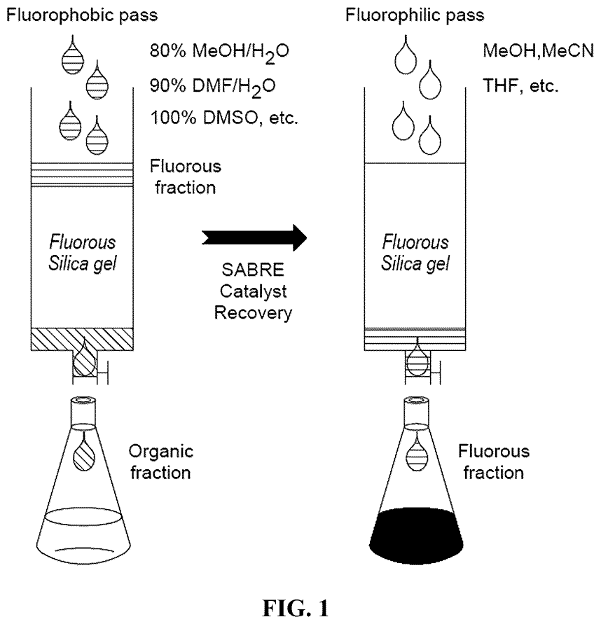

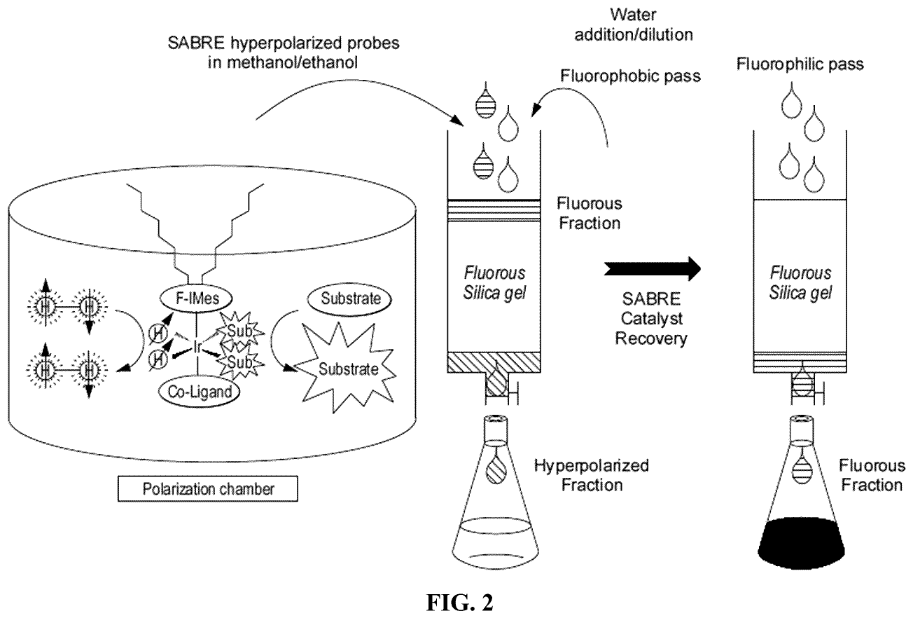

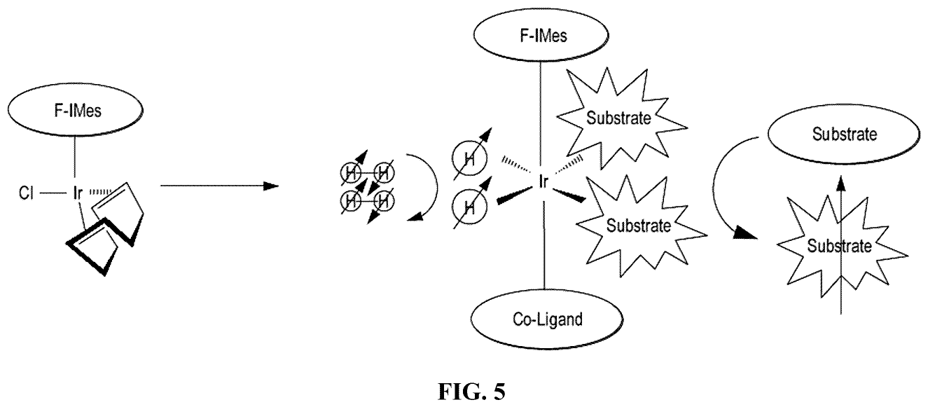

In some embodiments, the hyperpolarized substrate is isolated by treating the reaction mixture with a solid phase adsorbent to adsorb the perfluorinated SABRE catalyst, and recovering a liquid containing the hyperpolarized substrate, wherein the liquid is free (i.e., undetectable) or substantially free (e.g., less than 100 ppm, less than 50 ppm, less than 10 ppm, less than 5 ppm, or less than 1 ppm) of the perfluorinated SABRE catalyst. See, for example, FIG. 5. The solid phase adsorbent can be any suitable adsorbent capable of preferentially adsorbing the perfluorinated SABRE catalyst over the hyperpolarized substrate. For example the solid phase adsorbent can be a fluorous solid phase adsorbent, a reverse phase adsorbent (e.g., C18 adsorbents or the like), and polyethylene-based filters (e.g., ultrahigh molecular weight polyethylene). See, for example, FIGS. 1 and 2. In certain embodiments, the method of preparing a hyperpolarized substrate further comprises passing a fluorophobic solvent over the adsorbent and recovering an eluate containing the hyperpolarized substrate, wherein the eluate is free (i.e., undetectable) or substantially free (e.g., less than 100 ppm, less than 50 ppm, less than 10 ppm, less than 5 ppm, or less than 1 ppm) of the perfluorinated SABRE catalyst. The fluorophobic solvent can be any suitable solvent capable of preferentially washing the hyperpolarized substrate off of the solid phase adsorbent relative to the perfluorinated SABRE catalyst. For example, the fluorophobic solvent can comprise water and one or more of methanol, ethanol, acetonitrile, and dimethylformamide. Alternatively, or additionally, the method of preparing a hyperpolarized substrate can further comprise passing a fluorophilic solvent (e.g., a solvent comprising an organic solvent selected from methanol, ethanol, acetonitrile, THF, ethyl acetate, a chlorinated solvent (e.g., chlorinated alkanes such as methylene chloride, chloroform, and ethylene dichloride), a fluorinated solvent (e.g., fluorinated alkanes such as perfluorinated hexane and fluorinated ethers such as perfluorobutyl methyl ether), and a combination thereof) over the adsorbent, for example, to recover the perfluorinated SABRE catalyst. Exemplary fluorophilic solvent systems include mixtures of perfluorohexane, diethyl ether mixture, methoxy nonafluorobutane, and ethyl acetate.

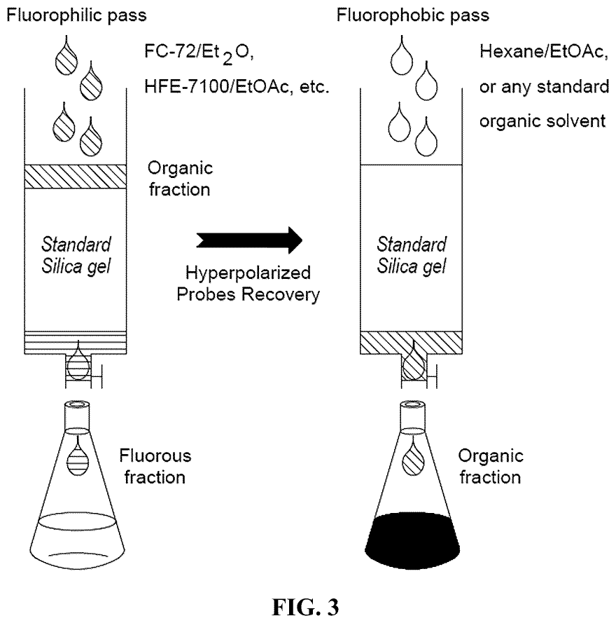

In some embodiments, the hyperpolarized substrate is isolated by treating the reaction mixture with a solid phase adsorbent to adsorb the hyperpolarized substrate, and recovering a liquid containing the perfluorinated SABRE catalyst, wherein the liquid is free (i.e., undetectable) or substantially free (e.g., less than 100 ppm, less than 50 ppm, less than 10 ppm, less than 5 ppm, or less than 1 ppm) of the hyperpolarized substrate. The solid phase adsorbent can be any suitable adsorbent capable of preferentially adsorbing the hyperpolarized substrate over the perfluorinated SABRE catalyst. For example the solid phase adsorbent can be a normal phase adsorbent such as, for example silica, alumina, or the like. See, for example, FIG. 3. In certain embodiments, the method of preparing a hyperpolarized substrate further comprises passing a fluorophilic solvent over the adsorbent and recovering an eluate containing the perfluorinated SABRE catalyst, wherein the eluate is free (i.e., undetectable) or substantially free (e.g., less than 100 ppm, less than 50 ppm, less than 10 ppm, less than 5 ppm, or less than 1 ppm) of the hyperpolarized substrate. The fluorophilic solvent can be any suitable solvent capable of preferentially washing the perfluorinated SABRE catalyst off of the solid phase adsorbent relative to the hyperpolarized substrate. For example, the fluorophilic solvent can comprise methanol, ethanol, acetonitrile, THF, ethyl acetate, a chlorinated solvent (e.g., chlorinated alkanes such as methylene chloride, chloroform, and ethylene dichloride), a fluorinated solvent (e.g., fluorinated alkanes such as perfluorinated hexane and fluorinated ethers such as perfluorobutyl methyl ether), or a combination thereof. Alternatively, or additionally, the method of preparing a hyperpolarized substrate can further comprise passing a fluorophobic solvent (e.g., a solvent comprising water, methanol, ethanol, acetonitrile, dimethylformamide, or a combination thereof) over the adsorbent, for example, to recover the hyperpolarized substrate.

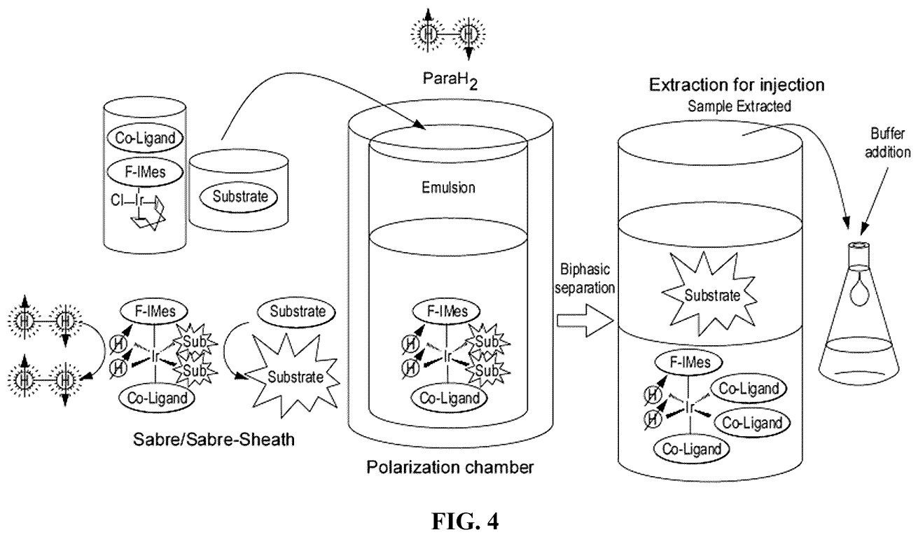

In any of the embodiments disclosed herein, the perfluorinated SABRE catalyst and/or the hyperpolarized substrate can exist in a monophasic or a biphasic mixture. The monophasic or biphasic mixture can comprise any combination of solvents described herein. For example, the biphasic mixture can comprise a polar solvent (e.g., water, methanol, and ethanol) in combination with a non-polar solvent (e.g., an organic solvent or a fluorous solvent). In embodiments, where the perfluorinated SABRE catalyst and/or the hyperpolarized substrate exists in a biphasic solvent, the perfluorinated SABRE catalyst and/or the hyperpolarized substrate can be isolated by a liquid/liquid extraction. See, for example, FIG. 4. Thus, in some embodiments, the hyperpolarized substrate is isolated by a liquid/liquid extraction, for example, by partitioning the perfluorinated SABRE catalyst and the hyperpolarized substrate between a methanolic mixture and a fluorous solvent or partitioning the perfluorinated SABRE catalyst and the hyperpolarized substrate between a methanolic mixture and an organic solvent.

In some embodiments, the hyperpolarized substrate is isolated by precipitating the perfluorinated SABRE catalyst and filtering and removing the precipitated perfluorinated SABRE catalyst from the hyperpolarized substrate. Typically, the perfluorinated SABRE catalyst is precipitated by addition of solvents in which the perfluorinated SABRE catalyst is not soluble (e.g., hexane, pentane, water, ethanol, or the like). In certain embodiments, the perfluorinated SABRE catalyst is precipitated by the addition of water.

The perfluorinated SABRE catalyst and/or the hyperpolarized substrate can be dried or concentrated (e.g., under reduced pressure, using a desiccant, heating, or a combination thereof). Alternatively, or additionally, the perfluorinated SABRE catalyst and/or the hyperpolarized substrate can be diluted or reconstituted with a solvent (e.g., water) to provide a desired concentration. For example, the perfluorinated SABRE catalyst can be isolated and re-used as a hyperpolarization catalyst. Similarly, the hyperpolarized substrates can be dried or concentrated to remove organic solvents and reconstituted in water for administration to a subject.

The substrate can be any compound comprising a 12 spin nucleus or nuclei. For example, the substrate can comprise ¹H, ¹³C, ¹⁵N, ¹⁹F, ³¹p ²⁹Si, or a combination thereof. In some embodiments, the substrate further comprises ²D. Thus, the methods described herein can be used to enhance the signal of ¹H, ¹³C, ¹⁵N, ¹⁹F, ³¹P and/or ²⁹Si response of a target substrate. Generally, the spin polarization transfer described herein is based on the SABRE effect; however the methods can be extended to parahydrogen—induced polarization (PHIP).

In some embodiments, the method further comprises recycling and reusing the perfluorinated SABRE catalyst more than one time (e.g., more than two times, more than three times, more than 4 times, more than 5 times, or more than 6 times). Thus, in some embodiments, the present invention provides a method of preparing a hyperpolarized substrate, the method comprising:
(i) providing a perfluorinated SABRE catalyst comprising a d-block element and a perfluorinated ligand, wherein the perfluorinated ligand is of Formula (I):

[Lₘ-(NHC)—(Y—Z)_{q}] Formula (I),
or a salt thereof, and wherein
   each L is independently selected from hydrogen, adamantyl, a substituted or unsubstituted aromatic group, or a substituted or unsubstituted heteroaromatic group,
   NHC is a 4 to 7-membered N-heterocyclic carbenyl group where NHC is bound to the d-block element via a carbene,
   each Y is independently selected from a bond or a spacer group,
   each Z is a perfluorinated tag,
   m is an integer from 1 to 4, and
   q is an integer from 1 to 3;
(ii) providing a co-ligand to interact with the perfluorinated SABRE catalyst to facilitate formation of an active perfluorinated SABRE catalyst;
(iii) combining the active perfluorinated SABRE catalyst with parahydrogen and a substrate comprising a ½ spin nucleus or nuclei in a solvent to obtain a first reaction mixture;
(iv) hyperpolarizing the first reaction mixture obtained in (iii) by exposing the mixture to a magnetic field or by radiofrequency excitation to obtain a hyperpolarized active perfluorinated SABRE catalyst-substrate and/or a hyperpolarized substrate;
   (v) performing an aqueous phase extraction and/or a fluorinated phase extraction to separate the hyperpolarized substrate from the perfluorinated SABRE catalyst;
   (vi) providing additional co-ligand to interact with the perfluorinated SABRE catalyst to reactivate the perfluorinated SABRE catalyst;
   (vii) combining the reactivated perfluorinated SABRE catalyst with additional parahydrogen and additional substrate comprising a ½ spin nucleus or nuclei in the solvent to obtain a second reaction mixture; and
   (viii) hyperpolarizing the second reaction mixture obtained in (vii) by exposing the mixture to a magnetic field or by radiofrequency excitation to obtain additional hyperpolarized active perfluorinated SABRE catalyst-substrate and/or additional hyperpolarized substrate. In other words, once the hyperpolarized substrate is separated from the perfluorinated SABRE catalyst, the perfluorinated SABRE catalyst can be reused to hyperpolarize additional substrate by repeating steps (ii)-(iv).

In some embodiments, the substrate is selected from ketoglutarate, ketoisocaproate, pyruvate, N-acetyl cysteine, and salts or esters thereof. In certain embodiments, the substrate is selected from 1-¹³C-ketoglutarate, 1-¹³C-5-¹²C-ketoglutarate, 1-¹³C-pyruvate, 1-¹³C-N-acetyl cysteine, ¹⁵N₂-isoniazid (or pyridyl-4-carbo-bis-¹⁵N₂-hydrazide), ¹³C₂,¹⁵N₃-metronidazole, ¹⁵N₂-1-aminoisoquinoline (1-AIQ), deuterated versions thereof, and salts thereof.

In some embodiments, the substrate is of Formula (II):

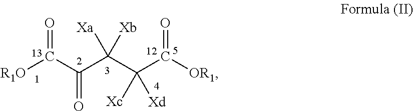

wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, provided that at least one of Xa, Xb, Xc, and Xd is deuterium,
or a pharmaceutically acceptable salt thereof.

Each R₁ may be independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl. In some embodiments, each R₁ is independently selected from a C₁-C₆ alkyl, for example, each R₁ can be methyl, ethyl, propyl (e.g., isopropyl or n-propyl), butyl (e.g., isobutyl, n-butyl, tert-butyl, or sec-butyl), pentyl, or hexyl. In some embodiments, each R₁ is independently selected from hydrogen, deuterium, and a cation. In embodiments where R₁ is a cation, it will be readily understood by a person of ordinary skill in the art that the compound of Formula (II) is a salt (e.g., a pharmaceutically acceptable salt) where the negative charge on oxygen is balanced by the cation. In certain embodiments, each R₁ independently is a cation or C₁-C₆ alkyl.

The present invention further provides a hyperpolarized substrate, or a pharmaceutically acceptable salt, obtained from any of the methods described herein, or a pharmaceutical composition comprising a hyperpolarized substrate, or a pharmaceutically acceptable salt, and a pharmaceutically acceptable carrier. In other words, the present invention provides imaging medium (e.g., an aqueous imaging composition) with enhanced sensitivity on a water-soluble compound comprising a hyperpolarizable nucleus or hyperpolarizable nuclei, which imaging medium is particularly well suited for nuclear magnetic resonance (NMR) spectroscopy and/or magnetic resonance imaging (MRI).

The present invention further provides a method of obtaining a magnetic resonance image of a tissue in a subject having or suspected to have a cancer or an adverse vascular condition comprising administering to the subject a hyperpolarized substrate described herein, or a pharmaceutical composition thereof, and imaging the subject by magnetic resonance imaging. In an aspect, the subject has a cancer such as, for example, a cancer is selected from breast cancer, colon cancer, rectal cancer, bladder cancer, endometrial cancer, kidney cancer, lung cancer, melanoma, non-Hodgkin lymphoma, pancreatic cancer, prostate cancer, and thyroid cancer. In another aspect, the subject has an adverse vascular condition such as, for example, a vascular condition selected from myocardial infarction, stroke, and pulmonary disease (e.g., COPD, lung fibrosis, long-term COVID-19 symptom, and combinations thereof).

In some embodiments, the invention provides a method of diagnosing or monitoring a patient having or suspected to have a cancer, the method comprising administering a hyperpolarized substrate or a pharmaceutical composition as described above and diagnosing or monitoring the patient by hyperpolarized ¹³C-MRI. For example, a hyperpolarized substrate can be used in the method of diagnosing or monitoring a patient having or suspected to have a cancer. In certain embodiments, the method or use comprises identifying a mutation or mutations responsible for the cancer. In certain embodiments, the method or use identifies an IDH1 mutation as being responsible for the cancer. In other words, the method or use can be used to identify whether the patient has a tumor, for example, an IDH1 mutation.

The present invention further provides a perfluorinated compound of Formula (III):

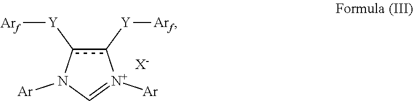

wherein
   each Ar is independently selected from a substituted or unsubstituted aromatic group or a substituted or unsubstituted heteroaromatic group,
   each Ar_{f} is independently selected from a perfluorinated substituted or unsubstituted aromatic group or a perfluorinated substituted or unsubstituted heteroaromatic group,
   each Y is independently selected from a bond or a spacer group,
   X is an anion, and
   [symbol] a single bond or a double bond.

All aspects of the perfluorinated compound of Formula (III) are as described with respect to any of Formulae (I) and (Ia)-(If). For example, Ar_{f} is independently selected from a perfluorinated substituted or unsubstituted aromatic group or a perfluorinated substituted or unsubstituted heteroaromatic group, wherein substituted or unsubstituted aromatic group and substituted or unsubstituted heteroaromatic group are as described with respect to any of Formulae (I) and (Ia)-(If). In some embodiments of the perfluorinated compound of Formula (III), (i) each Ar is independently selected from a substituted or unsubstituted aromatic group, (ii) each Ar_{f} is independently selected from a perfluorinated substituted or unsubstituted aromatic group, and/or (iii) each Y is independently selected from a spacer group selected from C₁₋₅ alkyl and C₁₋₅ heteroalkyl.

In certain embodiments, the perfluorinated compound is of Formula (IIIa):

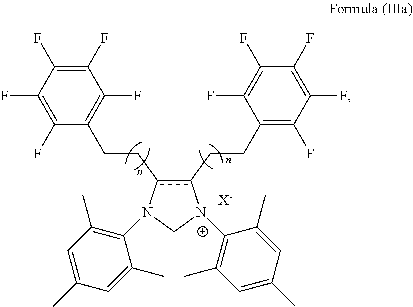

wherein
   each n independently is an integer from 0 to 4,
   X is an anion, and
   [symbol] is a single bond or a double bond.

In Formulae (III) and (IIIa), X is an anion. X can be any suitable anion. For example, X can be a halide ion (e.g., fluoride chloride, bromide, or iodide) or a fluorate ion (e.g., tetrafluoroborate or hexafluorophosphate).

The perfluorinated compound of Formula (III) or (IIIa) can be prepared by any suitable means. For example, the perfluorinated compound can be prepared by a method comprising:
(i) reacting an alpha-bromo ketone comprising a perfluorinated substituted or unsubstituted aromatic group or a perfluorinated substituted or unsubstituted heteroaromatic group with an amidine comprising a substituted or unsubstituted aromatic group or a substituted or unsubstituted heteroaromatic group in the presence of a base to form an alpha-amino ketone,
(ii) optionally reducing the alpha-amino ketone with a reducing agent to form an alpha-amino alcohol, and
(iii) cyclizing the alpha-amino ketone or the alpha-amino alcohol to form the perfluorinated compound.

The perfluorinated compound of Formula (III) or (IIIa) can be used in any suitable application. For example, the perfluorinated compound of Formula (III) or (IIIa) can be used for catalysis (e.g., polymerization catalysis or SABRE catalysis). Thus, in some embodiments, the invention provides a catalyst (e.g., an olefin metathesis catalyst or a SABRE catalyst) comprising a d-block element and a perfluorinated compound of Formula (III) or (IIIa) as a ligand, and a method of using the same. For example, the olefin metathesis catalyst, comprising a perfluorinated compound of Formula (III) or (IIIa), can be used in a method of polymerizing an olefin, the method comprising combining the olefin metathesis catalyst and an olefin in a reaction mixture. Similarly, the SABRE catalyst, comprising a perfluorinated compound of Formula (III) or (IIIa), can be used in a method of hyperpolarizing a substrate, as described herein.

The invention further provides a kit comprising a perfluorinated SABRE catalyst (e.g., the precatalyst), a co-ligand, and a solvent as defined herein. In some embodiments of the kit, the perfluorinated SABRE catalyst (e.g., the precatalyst) is separated from the co-ligand and/or the solvent such that the perfluorinated SABRE catalyst (e.g., the precatalyst) can be added to the hyperpolarizing system separately from the co-ligand and/or the solvent. In certain embodiments, the kit further comprises a substrate. In embodiments where the kit further comprises a substrate, the substrate can be (i) combined with the co-ligand and/or the solvent, but separate from the perfluorinated SABRE catalyst (e.g., the precatalyst), (ii) combined with the perfluorinated SABRE catalyst (e.g., the precatalyst), a co-ligand, and a solvent, or (iii) separated from the perfluorinated SABRE catalyst (e.g., the precatalyst), a co-ligand, and a solvent.

### Aspects of the Disclosure

Aspects, including embodiments, of the invention described herein may be beneficial alone or in combination, with one or more other aspects or embodiments. Without limiting the foregoing description, certain non-limiting aspects of the disclosure numbered 1-81 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) In aspect (1) is provided a method of preparing a hyperpolarized substrate, the method comprising:
(i) providing a perfluorinated SABRE catalyst comprising a d-block element and a perfluorinated ligand, wherein the perfluorinated ligand is of Formula (I):

[Lₘ-(NHC)—(Y—Z)_{q}] Formula (I),
or a salt thereof, and wherein
   each L is independently selected from hydrogen, adamantyl, a substituted or unsubstituted aromatic group, or a substituted or unsubstituted heteroaromatic group,
   NHC is a 4 to 7-membered N-heterocyclic carbenyl group where NHC is bound to the d-block element via a carbene,
   each Y is independently selected from a bond or a spacer group,
   each Z is a perfluorinated tag,
   m is an integer from 1 to 4, and
   q is an integer from 1 to 3;
   (ii) providing a co-ligand to interact with the perfluorinated SABRE catalyst to facilitate formation of an active perfluorinated SABRE catalyst;
   (iii) combining the active perfluorinated SABRE catalyst with parahydrogen and a substrate comprising a ½ spin nucleus or nuclei in a solvent to obtain a first reaction mixture;
   (iv) hyperpolarizing the first reaction mixture obtained in (iii) by exposing the mixture to a magnetic field or by radiofrequency excitation to obtain a hyperpolarized active perfluorinated SABRE catalyst-substrate and/or a hyperpolarized substrate;
   (v) performing an aqueous phase extraction and/or a fluorinated phase extraction to separate the hyperpolarized substrate from the perfluorinated SABRE catalyst;
   (vi) providing additional co-ligand to interact with the perfluorinated SABRE catalyst to reactivate the perfluorinated SABRE catalyst;
   (vii) combining the reactivated perfluorinated SABRE catalyst with additional parahydrogen and additional substrate comprising a ½ spin nucleus or nuclei in the solvent to obtain a second reaction mixture; and
   (viii) hyperpolarizing the second reaction mixture obtained in (vii) by exposing the mixture to a magnetic field or by radiofrequency excitation to obtain additional hyperpolarized active perfluorinated SABRE catalyst-substrate and/or additional hyperpolarized substrate.

(2) In aspect (2) is provided the method of aspect 1, wherein the substrate comprises ¹H, ¹³C, ¹⁵N, ¹⁹F, ³¹P ²⁹Si, or a combination thereof.

(3) In aspect (3) is provided the method of aspect 2, wherein the substrate further comprises 2D.

(4) In aspect (4) is provided the method of any one of aspects 1-3, wherein the co-ligand is a compound containing one or more sulfoxide groups, thioester groups, phosphine groups, amine groups, CO groups, isonitrile groups, nitrogen-containing heterocyclic groups, or a combination thereof.

(5) In aspect (5) is provided the method of any one of aspects 1-4, wherein the solvent comprises water, methanol, ethanol, a fluorous solvent, or a mixture thereof.

(6) In aspect (6) is provided the method of any one of aspects 1-5, wherein the solvent comprises a perfluoroalkane, diethyl ether, a nonafluorobutyl methyl ether, ethyl acetate, perfluorobutyl methyl ether, a fluorocarbon derivative of THF FC 75, a decafluoromethoxy trifluoromethyl pentane, a hexafluoro propanol, a perfluoromethyl cyclohexane, and a combination thereof.

(7) In aspect (7) is provided the method of any one of aspects 1-4, wherein the solvent comprises water, methanol, ethanol, a nonafluorobutyl methyl ether, deuterated variants thereof, or a mixture thereof.

(8) In aspect (8) is provided the method of any one of aspects 1-7, wherein the solvent is deuterated.

(9) In aspect (9) is provided the method of any one of aspects 1-8, wherein the co-ligand is dimethyl sulfoxide.

(10) In aspect (10) is provided the p method of any one of aspects 1-8, wherein the co-ligand is fluorinated.

(11) In aspect (11) is provided the method of aspect 10, wherein the co-ligand is phenyl trifluoromethyl sulfoxide, 2,2,2-trifluoroethyl methane sulfonate, or di-2,2,2-trifluoroethyl sulfoxide.

(12) In aspect (12) is provided the method of any one of aspects 1-11, wherein the substrate is selected from ketoglutarate, ketoisocaproate, pyruvate, N-acetyl cysteine, and salts or esters thereof.

(13) In aspect (13) is provided the method of any one of aspects 1-12, wherein the substrate is selected from 1-¹³C-ketoglutarate, 1-¹³C-5-¹²C-ketoglutarate, 1-¹³C-pyruvate, 1-¹³C-N-acetyl cysteine, ¹⁵N₂-isoniazid (or pyridyl-4-carbo-bis-¹⁵N₂-hydrazide), ¹³C₂,¹⁵N₃-metronidazole, ¹⁵N₂-1-aminoisoquinoline (1-AIQ), deuterated versions thereof, and salts thereof.

(14) In aspect (14) is provided the method of any one of aspects 1-12, wherein the substrate is of Formula (II):

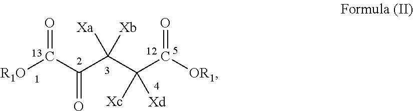

wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, provided that at least one of Xa, Xb, Xc, and Xd is deuterium,
or a pharmaceutically acceptable salt thereof.

(15) In aspect (15) is provided the method of any one of aspects 1-14, wherein NHC is a 5-membered N-heterocyclic carbenyl group.

(16) In aspect (16) is provided the method of aspect 15, wherein the 5-membered N-heterocyclic carbenyl group is imidazole-based, imidazoline-based, or thiazole-based.

(17) In aspect (17) is provided the method of any one of aspects 1-16, wherein NHC is a 4,5-disubstituted, a 1,3-disubstituted, or a 1,3,4,5-tetrasubstituted imidazole-based or imidazoline-based 5-membered N-heterocyclic carbenyl group.

(18) In aspect (18) is provided the method of any one of aspects 1-17, wherein NHC is a 4,5-disubstituted imidazolidinyl, a 1,3-disubstituted imidazolidinyl, a 1,3,4,5-tetrasubstituted imidazolidinyl, a 4,5-disubstituted 2,3-dihydro-imidazolyl, a 1,3-disubstituted 2,3-dihydro-imidazolyl, or a 1,3,4,5-tetrasubstituted 2,3-dihydro-imidazolyl.

(19) In aspect (19) is provided the method of any one of aspects 1-14, wherein the perfluorinated ligand is of Formula (Ia) or (Ib):

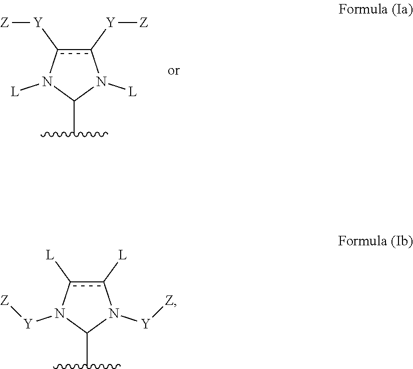

or a salt thereof, and wherein
   each L independently is hydrogen, adamantyl, a substituted or unsubstituted aromatic group, or a substituted or unsubstituted heteroaromatic group,
   each Y independently is a bond or a spacer group,
   each Z independently is a perfluorinated tag,
   [symbol] is a single bond or a double bond, and
   [symbol] represents the bond to the d-block element via the carbene.

(20) In aspect (20) is provided the method of any one of aspects 1-14, wherein the perfluorinated ligand is of Formula (Ic) or (Id):

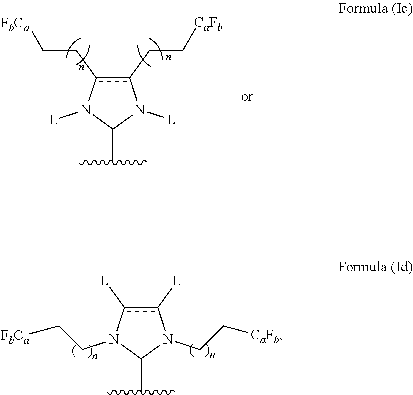

or a salt thereof, and wherein
   each L independently is hydrogen, adamantyl, a substituted or unsubstituted aromatic group, or a substituted or unsubstituted heteroaromatic group,
   a is 4 to 20,
   b=2a+1 or b=a−1,
   each n independently is an integer from 0 to 4,
   [symbol] is a single bond or a double bond, and
   [symbol] represents the bond to the d-block element via the carbene.

(21) In aspect (21) is provided the method of any one of aspects 1-14, wherein the perfluorinated ligand is of Formula (Ie) or (If):

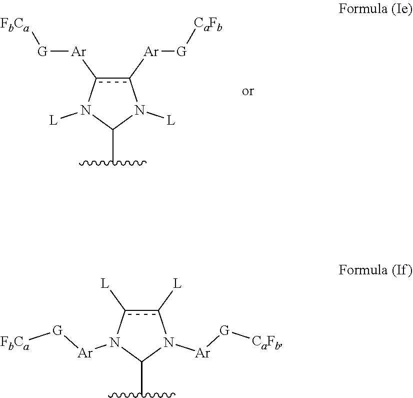

or a salt thereof, and wherein
   each L independently is hydrogen, adamantyl, a substituted or unsubstituted aromatic group, or a substituted or unsubstituted heteroaromatic group,
   each Ar independently is a substituted or unsubstituted aromatic group, or a substituted or unsubstituted heteroaromatic group,
   each G independently is a bond, C₁₋₆ alkyl, C₁₋₆ alkenyl, or C₁₋₆ heteroalkyl,
   a is 4 to 20,
   b=2a+1 or b=a−1,
   [symbol] is a single bond or a double bond, and
   [symbol] represents the bond to the d-block element via the carbene.

(22) In aspect (22) is provided the method of aspect 20 or aspect 21, wherein a is 4 to 10.

(23) In aspect (23) is provided the method of any one of aspects 1-22, where each L independently is hydrogen, adamantyl, 2-methylphenyl, 3-methylphenyl, 4-methylphenyl, 2,4-dimethylphenyl, 2,5-dimethylphenyl, 2,6-dimethylphenyl, 3,5-dimethylphenyl, 2,4,6-trimethylphenyl, 2-ethylphenyl, 3-ethylphenyl, 4-ethylphenyl, 2,4-diethylphenyl, 2,5-diethylphenyl, 2,6-diethylphenyl, 3,5-diethylphenyl, 2,4,6-triethylphenyl, 2-npropylphenyl, 3-npropylphenyl, 4-npropylphenyl, 2,4-di-npropylphenyl, 2,5-di-npropylphenyl, 2,6-di-npropylphenyl, 3,5-di-npropylphenyl, 2,4,6-tri-npropylphenyl, 2-isopropylphenyl, 3-isopropylphenyl, 4-isopropylphenyl, 2,4-di-isopropylphenyl, 2,5-di-isopropylphenyl, 2,6-di-isopropylphenyl, 3,5-di-isopropylphenyl, 2,4,6-tri-isopropylphenyl, 2-isobutylphenyl, 3-isobutylphenyl, 4-isobutylphenyl, 2,4-di-isobutylphenyl, 2,5-di-isobutylphenyl, 2,6-di-isobutylphenyl, 3,5-di-isobutylphenyl, 2,4,6-tri-isobutylphenyl, 2-secbutylphenyl, 3-secbutylphenyl, 4-secbutylphenyl, 2,4-di-secbutylphenyl, 2,5-di-secbutylphenyl, 2,6-di-secbutylphenyl, 3,5-di-secbutylphenyl, 2,4,6-tri-secbutylphenyl, 2-tbutylphenyl, 3-tbutylphenyl, 4-tbutylphenyl, 2,4-di-tbutylphenyl, 2,5-di-tbutylphenyl, 2,6-di-tbutylphenyl, 3,5-di-tbutylphenyl, 2,4,6-tri-tbutylphenyl, 2-cyclohexylphenyl, 3-cyclohexylphenyl, 4-cyclohexylphenyl, 2,4-di-cyclohexylphenyl, 2,5-di-cyclohexylphenyl, 2,6-di-cyclohexylphenyl, 3,5-di-cyclohexylphenyl, or 2,4,6-tri-cyclohexylphenyl.

(24) In aspect (24) is provided the method of any one of aspects 1-23, where each L independently is hydrogen or 2,4,6-trimethylphenyl.

(25) In aspect (25) is provided the method of any one of aspects 1-24, wherein each Y independently is a bond, a substituted or unsubstituted C₁₋₁₀ alkyl group, a substituted or unsubstituted C₂₋₁₀ alkenyl group, a substituted or unsubstituted C₂₋₁₀ alkynyl group, a substituted or unsubstituted C₁₋₁₀ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, or a linear or branched alkyleneoxy group.

(26) In aspect (26) is provided the method of any one of aspects 1-24, wherein each Y independently is a bond, a substituted or unsubstituted C₁₋₁₀ alkyl group, a substituted or unsubstituted C₂₋₁₀ alkenyl group, a substituted or unsubstituted aryl group, a substituted or unsubstituted heteroaryl group, a substituted or unsubstituted alkaryl group, or a substituted or unsubstituted arylalkyl group.

(27) In aspect (27) is provided the method of any one of aspects 1-24, wherein each Y independently is a bond or a substituted or unsubstituted C₁₋₁₀ alkyl group.

(28) In aspect (28) is provided the method of any one of aspects 1-27, wherein the perfluorinated tag is a perfluorinated C₃₋₆₀ group comprising only carbon and fluorine atoms.

(29) In aspect (29) is provided the method of any one of aspects 1-27, wherein the perfluorinated tag is a perfluorinated C₃₋₄₀ group comprising only carbon and fluorine atoms.

(30) In aspect (30) is provided the method of any one of aspects 1-27, wherein the perfluorinated tag is a perfluorinated C₃₋₂₀ group.

(31) In aspect (31) is provided the method of any one of aspects 1-27, wherein the perfluorinated tag is selected from a C₄F₉ group, a C₅F₁₁ group, a C₆F₁₃ group, a C₇F₁₅ group, a C₈F₁₇ group, a C₉F₁₉ group, a C₁₀F₂₁ group, a C₆F₅ group, C₄F₇ group, a C₅F₉ group, a C₆F₁₁ group, a C₇F₁₃ group, a C₈F₁₅ group, a C₉F₁₇ group, and a C₁₀F₁₉ group.

(32) In aspect (32) is provided the method of any one of aspects 1-14, wherein the perfluorinated ligand is

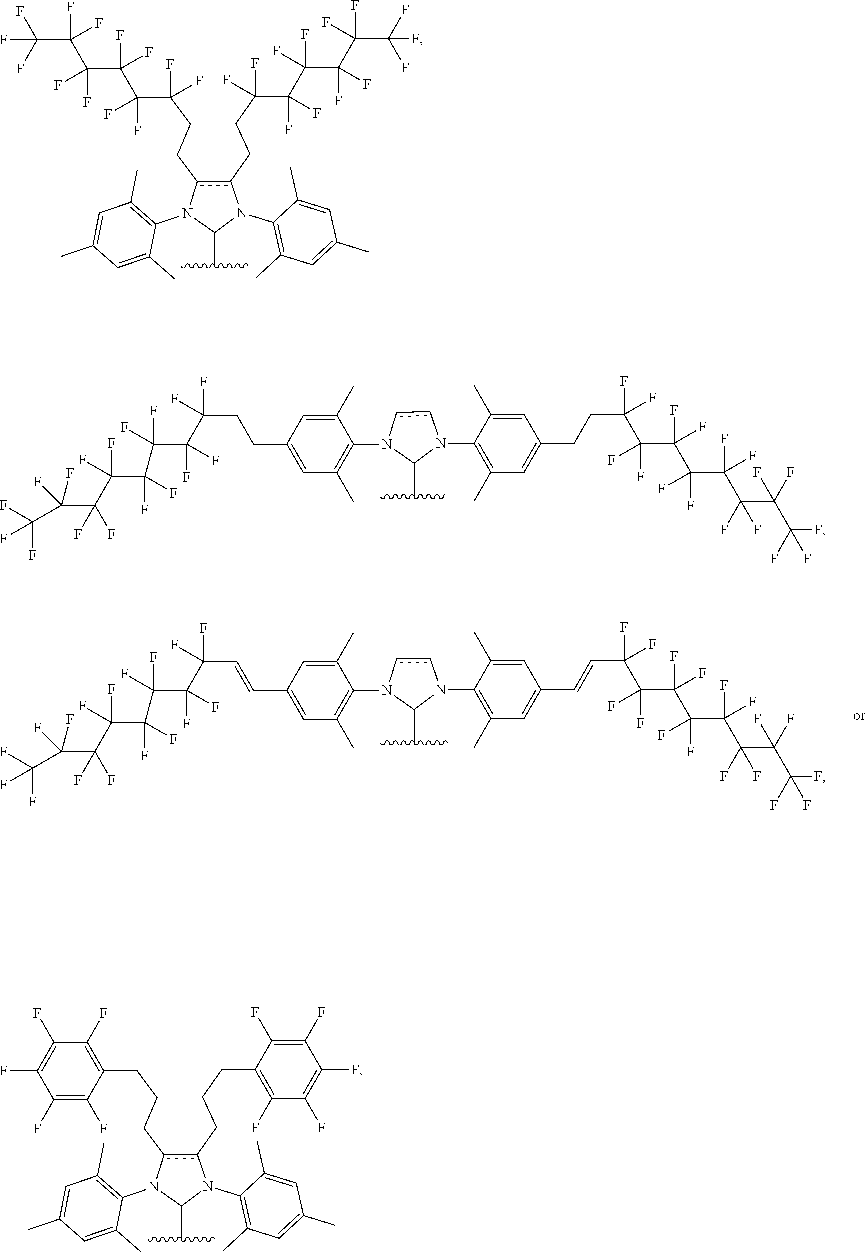

or a salt thereof, and wherein [symbol] is a single bond or a double bond, and [symbol] represents the bond to the d-block element via the carbene.

(33) In aspect (33) is provided the method of any one of aspects 1-32, wherein the d-block element is a transition metal.

(34) In aspect (34) is provided the method of any one of aspects 1-33, wherein the d-block element is Co, Rh, Ir, Ru, Pd, Pt, or Mt.

(35) In aspect (35) is provided the method of any one of aspects 1-34, wherein the SABRE catalyst further comprises an additional ligand.

(36) In aspect (36) is provided the method of aspect 35, wherein the SABRE catalyst further comprises an additional ligand selected from phosphine ligands, carbene ligands, imidazole ligands, pincer chelating ligands, and compounds comprising a sulfoxide group.

(37) In aspect (37) is provided a hyperpolarized substrate obtained from the method of any one of aspects 1-36, or a pharmaceutically acceptable salt thereof.

(38) In aspect (38) is provided a pharmaceutical composition comprising a hyperpolarized substrate of aspect 37, or a pharmaceutically acceptable salt thereof, and a pharmaceutically acceptable carrier.

(39) In aspect (39) is provided a method of obtaining a magnetic resonance image of a tissue in a subject having or suspected to have a cancer or an adverse vascular condition comprising administering to the subject a hyperpolarized substrate according to aspect 37 or a pharmaceutical composition according to aspect 38 and imaging the subject by magnetic resonance imaging.

(40) In aspect (40) is provided the method of aspect 39, wherein the subject has cancer.

(41) In aspect (41) is provided the method of aspect 40, wherein the cancer is selected from breast cancer, colon cancer, rectal cancer, bladder cancer, endometrial cancer, kidney cancer, lung cancer, melanoma, non-Hodgkin lymphoma, pancreatic cancer, prostate cancer, and thyroid cancer.

(42) In aspect (42) is provided the method of aspect 39, wherein the adverse vascular condition is selected from myocardial infarction, stroke, and pulmonary disease.

(43) In aspect (43) is provided the method of aspect 42, wherein the pulmonary disease is selected from COPD, lung fibrosis, long-term COVID-19 symptom, and a combination thereof.

(44) In aspect (44) is provided a kit comprising:
(i) a perfluorinated SABRE catalyst comprising a d-block element and a perfluorinated ligand, wherein the perfluorinated ligand is of Formula (I):

[Lₘ-(NHC)—(Y—Z)_{q}] Formula (I),

or a salt thereof, and wherein
each L is independently selected from hydrogen, adamantyl, a substituted or unsubstituted aromatic group, or a substituted or unsubstituted heteroaromatic group,
NHC is a 4 to 7-membered N-heterocyclic carbenyl group where NHC is bound to the d-block element via a carbene,
each Y is independently selected from a bond or a spacer group,
each Z is a perfluorinated tag,
m is an integer from 1 to 4, and
q is an integer from 1 to 3;
(ii) a co-ligand; and
(iii) a solvent.

(45) In aspect (45) is provided the kit of aspect 44, wherein NHC is a 5-membered N-heterocyclic carbenyl group.

(46) In aspect (46) is provided the kit of aspect 45, wherein the 5-membered N-heterocyclic carbenyl group is imidazole-based, imidazoline-based, or thiazole-based.

(47) In aspect (47) is provided the kit of any one of aspects 44-46, wherein NHC is a 4,5-disubstituted, a 1,3-disubstituted, or a 1,3,4,5-tetrasubstituted imidazole-based or imidazoline-based 5-membered N-heterocyclic carbenyl group.

(48) In aspect (48) is provided the kit of any one of aspects 44-47, wherein NHC is a 4,5-disubstituted imidazolidinyl, a 1,3-disubstituted imidazolidinyl, a 1,3,4,5-tetrasubstituted imidazolidinyl, a 4,5-disubstituted 2,3-dihydro-imidazolyl, a 1,3-disubstituted 2,3-dihydro-imidazolyl, or a 1,3,4,5-tetrasubstituted 2,3-dihydro-imidazolyl.

(49) In aspect (49) is provided the kit of any one of aspects 44-48, wherein the perfluorinated ligand is of Formula (Ia) or (Ib):

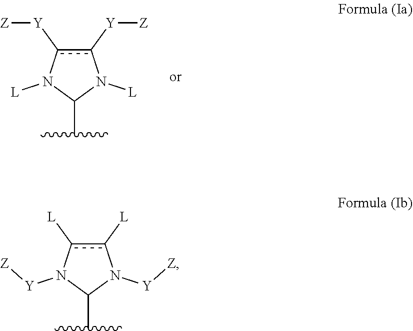

or a salt thereof, and wherein
   each L independently is hydrogen, adamantyl, a substituted or unsubstituted aromatic group, or a substituted or unsubstituted heteroaromatic group,
   each Y independently is a bond or a spacer group,
   each Z independently is a perfluorinated tag,
   [symbol] is a single bond or a double bond, and
   [symbol] represents the bond to the d-block element via the carbene.

(50) In aspect (50) is provided the kit of any one of aspects 44-48, wherein the perfluorinated ligand is of Formula (Ic) or (Id):

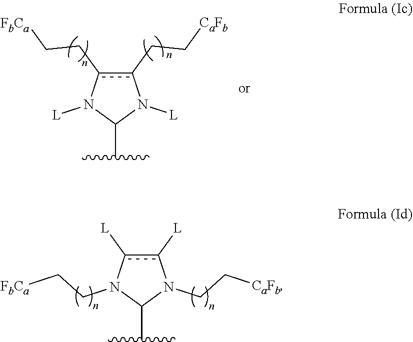

or a salt thereof, and wherein
   each L independently is hydrogen, adamantyl, a substituted or unsubstituted aromatic group, or a substituted or unsubstituted heteroaromatic group,
   a is 4 to 20,
   b=2a+1 or b=a−1,
   each n independently is an integer from 0 to 4,
   [symbol] is a single bond or a double bond, and
   [symbol] represents the bond to the d-block element via the carbene.

(51) In aspect (51) is provided the kit of any one of aspects 44-48, wherein the perfluorinated ligand is of Formula (Ie) or (If):

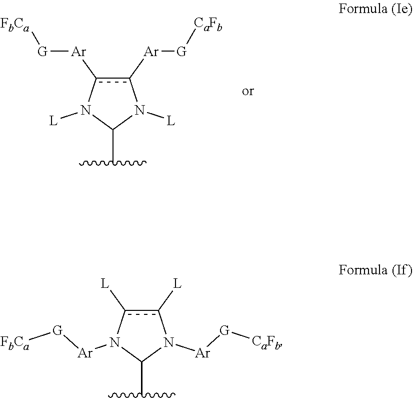

or a salt thereof, and wherein
   each L independently is hydrogen, adamantyl, a substituted or unsubstituted aromatic group, or a substituted or unsubstituted heteroaromatic group,
   each Ar independently is a substituted or unsubstituted aromatic group, or a substituted or unsubstituted heteroaromatic group,
   each G independently is a bond, C₁₋₆ alkyl, C₁₋₆ alkenyl, or C₁₋₆ heteroalkyl,
   a is 4 to 20,
   b=2a+1 or b=a−1,
   [symbol] is a single bond or a double bond, and
   [symbol] represents the bond to the d-block element via the carbene.

(52) In aspect (52) is provided the kit of aspect 51 or aspect 52, wherein a is 4 to 10.

(53) In aspect (53) is provided the kit of any one of aspects 44-52, where each L independently is hydrogen, adamantyl, 2-methylphenyl, 3-methylphenyl, 4-methylphenyl, 2,4-dimethylphenyl, 2,5-dimethylphenyl, 2,6-dimethylphenyl, 3,5-dimethylphenyl, 2,4,6-trimethylphenyl, 2-ethylphenyl, 3-ethylphenyl, 4-ethylphenyl, 2,4-diethylphenyl, 2,5-diethylphenyl, 2,6-diethylphenyl, 3,5-diethylphenyl, 2,4,6-triethylphenyl, 2-npropylphenyl, 3-npropylphenyl, 4-npropylphenyl, 2,4-di-npropylphenyl, 2,5-di-npropylphenyl, 2,6-di-npropylphenyl, 3,5-di-npropylphenyl, 2,4,6-tri-npropylphenyl, 2-isopropylphenyl, 3-isopropylphenyl, 4-isopropylphenyl, 2,4-di-isopropylphenyl, 2,5-di-isopropylphenyl, 2,6-di-isopropylphenyl, 3,5-di-isopropylphenyl, 2,4,6-tri-isopropylphenyl, 2-isobutylphenyl, 3-isobutylphenyl, 4-isobutylphenyl, 2,4-di-isobutylphenyl, 2,5-di-isobutylphenyl, 2,6-di-isobutylphenyl, 3,5-di-isobutylphenyl, 2,4,6-tri-isobutylphenyl, 2-secbutylphenyl, 3-secbutylphenyl, 4-secbutylphenyl, 2,4-di-secbutylphenyl, 2,5-di-secbutylphenyl, 2,6-di-secbutylphenyl, 3,5-di-secbutylphenyl, 2,4,6-tri-secbutylphenyl, 2-tbutylphenyl, 3-tbutylphenyl, 4-tbutylphenyl, 2,4-di-tbutylphenyl, 2,5-di-tbutylphenyl, 2,6-di-tbutylphenyl, 3,5-di-tbutylphenyl, 2,4,6-tri-tbutylphenyl, 2-cyclohexylphenyl, 3-cyclohexylphenyl, 4-cyclohexylphenyl, 2,4-di-cyclohexylphenyl, 2,5-di-cyclohexylphenyl, 2,6-di-cyclohexylphenyl, 3,5-di-cyclohexylphenyl, or 2,4,6-tri-cyclohexylphenyl.

(54) In aspect (54) is provided the kit of any one of aspects 44-53, where each L independently is hydrogen or 2,4,6-trimethylphenyl.

(55) In aspect (55) is provided the kit of any one of aspects 44-54, wherein each Y independently is a bond, a substituted or unsubstituted C₁₋₁₀ alkyl group, a substituted or unsubstituted C₂₋₁₀ alkenyl group, a substituted or unsubstituted C₂₋₁₀ alkynyl group, a substituted or unsubstituted C₁₋₁₀ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, or a linear or branched alkyleneoxy group.

(56) In aspect (56) is provided the kit of any one of aspects 44-55, wherein each Y independently is a bond, a substituted or unsubstituted C₁₋₁₀ alkyl group, a substituted or unsubstituted C₂₋₁₀ alkenyl group, a substituted or unsubstituted aryl group, a substituted or unsubstituted heteroaryl group, a substituted or unsubstituted alkaryl group, or a substituted or unsubstituted arylalkyl group.

(57) In aspect (57) is provided the kit of any one of aspects 44-52, wherein each Y independently is a bond or a substituted or unsubstituted C₁₋₁₀ alkyl group.

(58) In aspect (58) is provided the kit of any one of aspects 44-57, wherein the perfluorinated tag is a perfluorinated C₃₋₆₀ group comprising only carbon and fluorine atoms.

(59) In aspect (59) is provided the kit of any one of aspects 44-57, wherein the perfluorinated tag is a perfluorinated C₃₋₄₀ group comprising only carbon and fluorine atoms.

(60) In aspect (60) is provided the kit of any one of aspects 44-57, wherein the perfluorinated tag is a perfluorinated C₃₋₂₀ group.

(61) In aspect (61) is provided the kit of any one of aspects 44-60, wherein the perfluorinated tag is selected from a C₄F₉ group, a C₅F₁₁ group, a C₆F₁₃ group, a C₇F₁₅ group, a C₈F₁₇ group, a C₉F₁₉ group, a C₁₀F₂₁ group, a C₆F₅ group, C₄F₇ group, a C₅F₉ group, a C₆F₁₁ group, a C₇F₁₃ group, a C₈F₁₅ group, a C₉F₁₇ group, and a C₁₀F₁₉ group.

(62) In aspect (62) is provided the kit of any one of aspects 44-61, wherein the perfluorinated ligand is

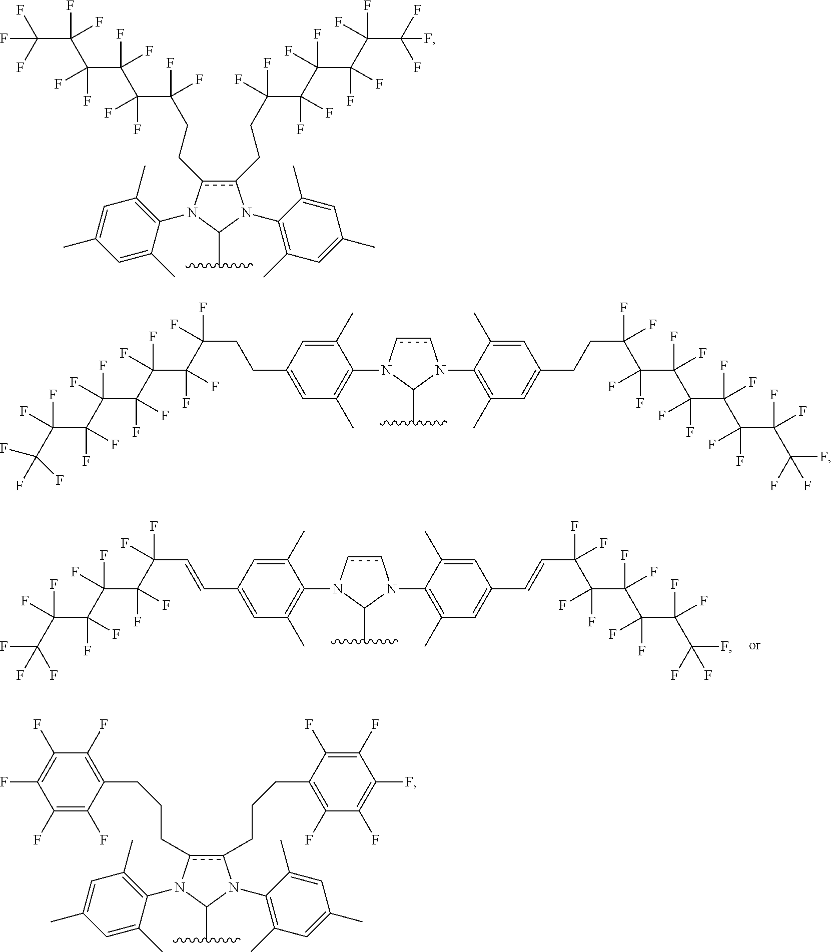

or a salt thereof, and wherein [symbol] is a single bond or a double bond, and [symbol] represents the bond to the d-block element via the carbene.

(63) In aspect (63) is provided the kit of any one of aspects 44-62, wherein the d-block element is a transition metal.

(64) In aspect (64) is provided the kit of any one of aspects 44-63, wherein the d-block element is Co, Rh, Ir, Ru, Pd, Pt, or Mt.

(65) In aspect (65) is provided the kit of any one of aspects 44-64, wherein the SABRE catalyst further comprises an additional ligand.

(66) In aspect (66) is provided the kit of aspect 65, wherein the SABRE catalyst further comprises an additional ligand selected from phosphine ligands, carbene ligands, imidazole ligands, pincer chelating ligands, and compounds comprising a sulfoxide group.

(67) In aspect (67) is provided the kit of any one of aspects 44-66, wherein the co-ligand is a compound containing one or more sulfoxide groups, thioester groups, phosphine groups, amine groups, CO groups, isonitrile groups, nitrogen-containing heterocyclic groups, or a combination thereof.

(68) In aspect (68) is provided the kit of any one of aspects 44-67, wherein the co-ligand is dimethyl sulfoxide.

(69) In aspect (69) is provided the kit of any one of aspects 44-67, wherein the co-ligand is fluorinated.

(70) In aspect (70) is provided the kit of aspect 69, wherein the co-ligand is phenyl trifluoromethyl sulfoxide, 2,2,2-trifluoroethyl methane sulfonate, or di-2,2,2-trifluoroethyl sulfoxide.

(71) In aspect (71) is provided the kit of any one of aspects 44-70, wherein the solvent comprises water, methanol, ethanol, a fluorous solvent, or a mixture thereof.

(72) In aspect (72) is provided the kit of any one of aspects 44-71, wherein the solvent comprises a perfluoroalkane, diethyl ether, a nonafluorobutyl methyl ether, ethyl acetate, perfluorobutyl methyl ether, a fluorocarbon derivative of THF FC 75, a decafluoromethoxy trifluoromethyl pentane, a hexafluoro propanol, a perfluoromethyl cyclohexane, and a combination thereof.

(73) In aspect (73) is provided the kit of any one of aspects 44-71, wherein the solvent comprises water, methanol, ethanol, a nonafluorobutyl methyl ether, deuterated variants thereof, or a mixture thereof.

(74) In aspect (74) is provided the kit of any one of aspects 44-73, wherein the solvent is deuterated.

(75) In aspect (75) is provided the kit of any one of aspects 44-74, wherein the kit further comprises a substrate.

(76) In aspect (76) is provided the kit of aspect 75, wherein the substrate comprises ¹H, ¹³C, ¹⁵N, ¹⁹F, ³¹P ²⁹Si, or a combination thereof.

(77) In aspect (77) is provided the kit of aspect 76, wherein the substrate further comprises ²D.

(78) In aspect (78) is provided the kit of any one of aspects 75-77, wherein the substrate is selected from ketoglutarate, ketoisocaproate, pyruvate, N-acetyl cysteine, and salts or esters thereof.

(79) In aspect (78) is provided the kit of any one of aspects 75-78, wherein the substrate is selected from 1-¹³C-ketoglutarate, 1-¹³C-5-¹²C-ketoglutarate, 1-¹³C-pyruvate, 1-¹³C-N-acetyl cysteine, ¹⁵N₂-isoniazid (or pyridyl-4-carbo-bis-¹⁵N₂-hydrazide), ¹³C₂,¹⁵N₃-metronidazole, ¹⁵N₂-1-aminoisoquinoline (1-AIQ), deuterated versions thereof, and salts thereof.

(80) In aspect (80) is provided the kit of any one of aspects 75-79, wherein the substrate is of Formula (II):

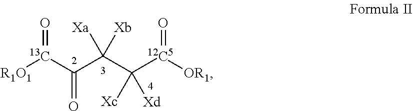

wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, provided that at least one of Xa, Xb, Xc, and Xd is deuterium,
or a pharmaceutically acceptable salt thereof.

(81) In aspect (81) is provided the kit of any one of aspect 44-80, wherein the perfluorinated SABRE catalyst is separated from the co-ligand and/or the solvent.

The following examples further illustrate the invention but, of course, should not be construed as in any way limiting its scope.

### Example 1

This example illustrates a method of preparing 1H,1H,2H,2H-perfluorooctyl-N,N′-bis(2,4,6-trimethylphenyl)-9,10-diamine:

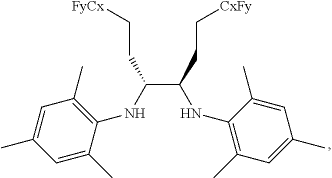

wherein x=6 and y=13.

A 1.7 M solution of tert-butyl lithium in pentane (5 mL, 9 mmol, 8 equiv.) was added to a solution of 1H,1H,2H,2H-perfluorooctyl iodide (2 g, 4 mmol, 4 equiv.) in dry Et₂O (60 mL) at −78° C. After the mixture had been stirred for 20 min at −78° C., the solid N,N′-dimesitylethanediimine (0.30 g, 1.03 mmol, 1 equiv.) was added portion wise. The reaction mixture was stirred for 4 h. The reaction was slowly warmed to −30° C. and quenched with a saturated solution of ammonium chloride (0.6 mL).

Water (20 mL) was added, the organic layer was separated, and the aqueous layer was extracted with diethyl ether (3×15 mL). The combined organic layers were dried over anhydrous MgSO₄, and concentrated. The crude product was purified by flash column chromatography (4:1 hexane/dichloromethane (DCM)) to yield diamine, diimine, and threo-diamine (0.6 g, 60% yield, white solid). ¹H NMR (400 MHz, CDCl₃) δ 1.64-1.82 (m, 2H, CH₂CHHCH(NHAr)), 2.00 (s, 12H, o-CH₃), 2.07˜2.27 (m, 4H,CHHCHHCH(NHAr)), 2.33 (s, 6H, p-CH₃), 2.67˜2.91 (m, 2H, CHHCH₂CH(NHAr)), 2.95 (br s, 2H, NH), 3.23 (br d, ³J_{H-H}=10 Hz, 2H, CH), 6.82 (s, 4H, Ar—CH) ppm. ¹⁹F NMR (282.23 MHz, CDCl₃) δ -83.8 (t, ³J_{F-F}=10 Hz, 6F, CF₃), −114.4 (m, 4F, CF₂CH₂), −121.9 (m, 4F, CF₂CF₂CH₂), −122.8 (m, 4F, CF₃CF₂CF₂CF₂), −123.4 (m, 4F, CF₃CF₂CF₂), −126.1 (m, 4F, CF₃CF₂) ppm. ¹³C NMR (400 MHz, CDCl₃) δ 18.3 (o —CH₃), 20.3 (p —CH₃), 21.7 (m, CF₂CH₂CH₂), 29.2 (t, ²J_{F-C}=22.2 Hz, CF₂CH₂), 57.1 (CH), 107-115 (m, 8 CF₂), 117.4 (qt, ¹J_{F-C}=288.3 Hz, ²J_{F-C}=33.3 Hz, CF₃), 118.7 (tt, ¹J_{F-C}=255.1 Hz, ²J_{F-C}=31.0 Hz, CF₂CH₂), 128.6 (Ar—C), 129.9 (Ar—CH), 131.4 (Ar—C),140.4 (Ar—C) ppm. MS (ESI), m/z (%): 990 [M+H]⁺(100).

### Example 2

This example illustrates the synthesis of trans-4,5-bis(3,3,4,4,5,5,6,6,7,7,8,8,8-tridecafluorooctyl)-1,3-bis(2,4,6-trimethylphenyl)-4,5-dihydroimidazolium tetrafluoroborate:

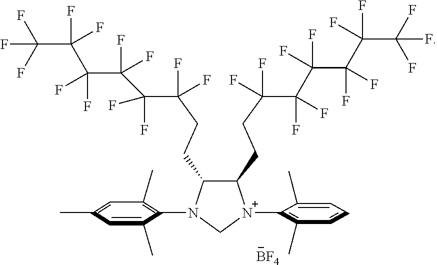

A mixture of 1H,1H,2H,2H-Perfluorooctyl-N,N′-bis(2,4,6-trimethylphenyl)-9,10-diamine (0.250 g, 0.25 mmol) from Example 1, ammonium tetrafluoroborate (-10% molar excess) (0.030 g, 0.25 mmol), and triethyl orthoformate (0.5 mL) was heated to 125° C. and stirred for 15 h. After cooling to room temperature, the solution was evaporated and the solid was triturated with diethyl ether (6×3 mL). The residue was redissolved in acetone, filtered, and concentrated to yield to the dihydroimidazolium tetrafluoroborate. (0.2 g, 70% yield, yellowish solid). ¹H NMR (400 MHz, acetone-d₆) δ 2.15˜2.70 (m, 8H, CF₂CH₂CH₂), 2.34 (s, 6H, p-CH₃), 2.51 (s, 6H, o-CH₃), 2.94 (br s, 2H, NH), 5.16 (m, 2H, CH₂CH₂CH), 7.17 (s, 4H, Ar—CH), 9.06 (s, 1H, N—CH═N) ppm. ¹⁹F NMR (400 MHz, acetone-d₆) δ-81.6 (t, ⁴J_{F-F}=10 Hz, 6F, CF₃), −115.1 (m, 4F, CF₂CF₂CH₂), −122.5 (m, 4F, CF₂CF₂CH₂), −123.6 (m, 4F, CF₃CF₂CF₂CF₂), −124.5 (m, 4F, CF₃CF₂CF₂), −126.9 (m, 4F, CF₃CF₂CF₂), −150.9 (4F, BF₄) ppm. ¹³C NMR (400 MHz, acetone-d₆) δ 17.4 (o —CH₃), δ 17.7 (o —CH₃), 19.9 (p —CH₃), 24.4 (m, CF₂CH₂CH₂), 26.2 (t, ²J_{F-C}=22.2 Hz, CF₂CH₂), 67.9 (CH), 105-118 (m, 8 CF₂), 118 (qt, ¹J_{F-C}=288.3 Hz, ²J_{F-C}=33.3 Hz, CF₃), 118.9 (tt, ¹J_{F-C}=254 Hz, ²J_{F-C}=31.0 Hz, CF₂CH₂), 129.5 (Ar—C), 130.1 (Ar—CH), 130.5 (Ar—CH), 135.8 (Ar—C), 136 (Ar—C),140.8 (Ar—C) ppm, 159.7 (N—C═N) ppm. MS (ESI) m/z (%): 999 [M+H]⁺(100).

### Example 3

This example illustrates the synthesis of trans-4,5-Bis(3,3,4,4,5,5,6,6,7,7,8,8,8-tridecafluorooctyl)-1,3bis(2,4,6-trimethylphenyl)-4,5-dihydroimidazolium chloride:

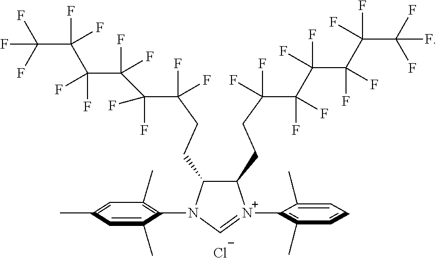

Dihydroimidazolidium tetrafluoroborate salt (0.5 g, 0.46 mmol) was dissolved in MeOH (1.5 mL) and passed through a short column of ion exchange resin Amberlite 400. The column was washed with MeOH until no spot was visible via TLC under UV. The solvent was removed, and the resulting yellowish solid was dried with a vacuum pump to yield product (0.48 g, 99%). ¹H NMR (400 MHz, acetone-d₆) δ 2.15˜2.70 (m, 8H, CF₂CH₂CH₂), 2.34 (s, 6H, p-CH₃), 2.51 (s, 6H, o-CH₃), 2.94 (br s, 2H, NH), 5.16 (m, 2H, CH₂CH₂CH), 7.17 (s, 4H, Ar—CH), 9.06 (s, 1H, N—CH═N) ppm. ¹⁹F NMR (400 MHz, acetone-d₆) δ-81.8 (t, ⁴J_{F-F}=10 Hz, 6F, CF₃), −115.1 (m, 4F, CF₂CF₂CH₂), −122.5 (m, 4F, CF₂CF₂CH₂), −123.6 (m, 4F, CF₃CF₂CF₂CF₂), −124.5 (m, 4F, CF₃CF₂CF₂), −127 (m, 4F,CF₃CF₂CF₂) ppm. ¹³C NMR (400 MHz, acetone-d₆) δ 17.4 (o —CH₃), δ 17.7 (o —CH₃), 19.9 (p —CH₃), 24.4 (m, CF₂CH₂CH₂), 26.2 (t, ²J_{F-C}=22.2 Hz, CF₂CH₂), 67.9 (CH), 105-118 (m, 8 CF₂), 118 (qt, ¹J_{F-C}=288.3 Hz, ²J_{F-C}=33.3 Hz, CF₃), 118.9 (tt, ¹J_{F-C}=254 Hz, ²J_{F-C}=31.0 Hz, CF₂CH₂), 129.5 (Ar—C), 130.1 (Ar—CH), 130.5 (Ar—CH), 135.8 (Ar—C), 136 (Ar—C),140.8 (Ar—C) ppm, 159.7 (N—C═N) ppm. MS (ESI) m/z (%): 999 [M+H]⁺ (100).

### Example 4

This example illustrates a method of synthesis of a fluorinated SABRE catalyst containing a transition metal in accordance with an aspect of the invention.

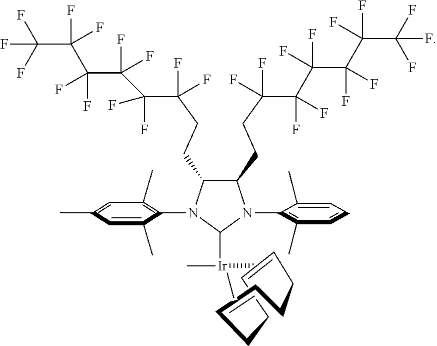

Potassium tert-butoxide (112 mg, 1.00 mmol, 2.5 eq.) was added to a stirred solution of trans-4,5-bis(3,3,4,4,5,5,6,6,7,7,8,8,8-tridecafluorooctyl)-1,3-bis(2,4,6-trimethylphenyl)-4,5-dihydroimidazolium chloride (320 mg, 0.88 mmol, 2.2 eq.) from Example 3 in tetrahydrofuran (10 mL) at room temperature in a glove box. The resulting suspension was stirred for 30 minutes. A solution of [Ir(COD)Cl]₂ (268 mg, 0.40 mmol, 1.0 eq.) was added and the resulting solution was stirred at room temperature overnight (Cowley et al., J Am. Chem. Soc., 133, 6134-6137 (2011)). The solvent was removed under reduced pressure to give the crude product and dried overnight under vacuum. This sample was dissolved in hexane and added to a 60 mL filter packed with SiO₂ gel in hexane. The crude solution was absorbed on the top of the silica gel, then hexane was added, to elute the compound, to give 104 mg (40% yield). ¹H NMR (400 MHz, CDCl₃) δ 1.04, 1.18, 1.49, 1.76, 2.13, 2.22, 2.25, 2.4, 2.48, 2.66, 2.94, 3.73, 4.01, 4.2, 6.84, 6.91 6.97 ppm. ¹⁹F NMR (400 MHz, CDCl₃) δ −81.08, −114.33, −122.05, −123.07, −124.02, −126.39 ppm. ¹³C NMR (100 MHz, CDCl₃) δ 18.7, 20.95, 21.07, 21.34, 26.45, 27.21, 29.83, 30.74, 31.42, 49.93, 55.12, 68.11, 68.47, 83.26, 86.69, 108.32, 111.0, 112.95, 115.85, 117.75, 118.69, 129.02, 129.3, 130.36, 130.55, 134.71, 134.8, 135.32, 136.33, 137.92, 138.28, 138.45, 138.5, 206.72 ppm. MS (ESI) m/z (%): 1299 [M+H]⁺(100).

### Example 5

This example illustrates a method of hyperpolarizing a [1-¹³C]pyruvate in accordance with an aspect of the invention, as shown in Scheme 1.

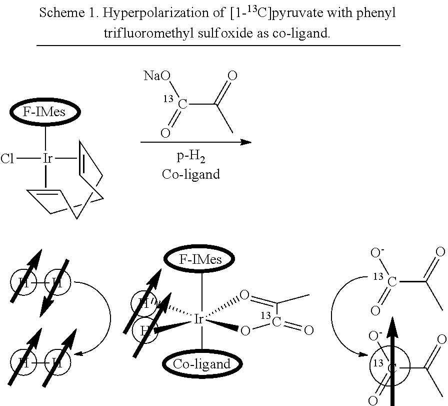

wherein co-ligand=phenyl trifluoromethyl sulfoxide.

In the reaction scheme above, hyperpolarization of [1-¹³C]pyruvate was performed using SABRE in SHield Enabled Alignment Transfer to Heteronuclei (SABRE-SHEATH) (Theis et al., J Am. Chem. Soc., 137, 1404-1407 (2015) and Truong et al., J Phys. Chem. C, 119, 8786-8797 (2015)) tailored for the ¹³C nucleus (Barskiy et al., ChemPhysChem, 18, 1493-1498 (2017)) using the co-ligand approach developed by Duckett and co-workers (Iali et al., Angew. Chemie —Int. Ed., 58, 10271-10275 (2019)). Sodium [1-¹³C]-pyruvate and deuterated methanol-d₄ solvent were purchased from Sigma-Aldrich and used without any further purification. The [IrCl(COD)(F-IMes)] SABRE catalyst used for this Example was prepared according to Example 4. The active catalyst used herein was prepared with a fixed ratio of substrate to Ir(F-IMes) SABRE catalyst of Example 4, and phenyl trifluoromethyl sulfoxide (PTFSO) in 0.6 mL of methanol-d₄ in a 5 mm NMR tube.

Parahydrogen was generated using a Gas-Delivery Manifold. Ultra-high-purity hydrogen gas (Airgas) was fed into a ParaHydrogen flow cryostat (Xeus technology LTD) and enriched to about 50% parahydrogen in the presence of a spin-exchange catalyst (Fe₂O₃) at liquid nitrogen temperature (77K). The p-H₂ flow was directed via PTFE tubing to a mass flow controller (MFC, Sierra Instruments SmartTrak 100 series) set at 90 scc/m and directed to a conventional 5 mm NMR tube (Norell) to allow bubbling through the sample. The entire pH₂ line was pressurized to 100 psi.

The magnetic shield condition was as follows. Magnetic fields near or below ˜1 μT were achieved with an apparatus consisting of a solenoid coil placed inside a mu-metal shield (Magnetic Shield Corporation, model No. ZG-206). The shield was degaussed using internal homebuilt coils driven by a Variac when necessary. The solenoid had a 41 mm diameter (40 mm core, 20 cm long windings with 220 turns AWG20 (0.9 mm) Cu wire and with 220Ω resistor in series. The solenoid coil was driven by commercial 1.5V batteries with a variable-resistance decade box in series to provide finer control of the internal magnetic field inside the shield. Typical values of the field within the shield were between ±1.2μT, with SABRE SHEATH experiments typically between −0.7 μT and +0.8 μT in the sample region. The values were monitored between SABRE experiments using a Lakeshore Cryotronics Gaussmeter (Model No. 475 DSP with HMMA-2512-VR Hall Probe).

MR experiments were performed using a 1 T Magritek Spinsolve benchtop NMR spectrometer. All ¹³C NMR spectra were taken with ¹H decoupling turned off throughout the duration of the experiment. Time required to manually transfer the sample from the shield region to the magnet for low-field NMR acquisition was usually <5 s.

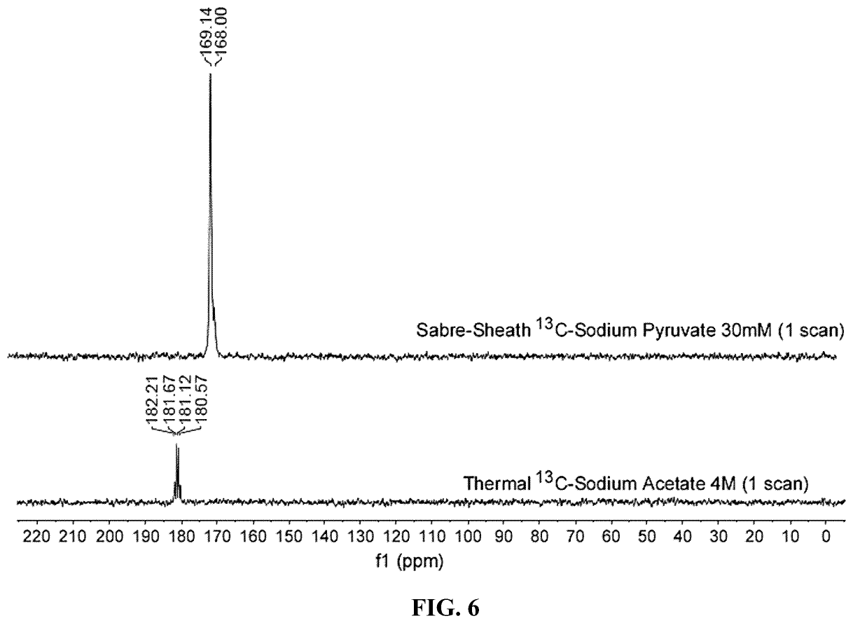

The efficient hyperpolarization transfer from nascent p-H₂-derived hydrides to the ¹³C nuclear spin of [1-¹³C]pyruvate was attained by performing SABRE in sub-microtesla magnetic fields using SABRE in SHield Enables Alignment Transfer to Heteronuclei (SABRE-SHEATH) using a solution mixture of [IrCl(H)₂(PTFSO)₂(F-IMes)], [1-¹³C]pyruvate) and p-H₂ in deuterated methanol. FIG. 6 depicts, in the top curve, a single-scan HP ¹³C spectrum obtained for the hyperpolarized probe. The bottom curve shows a single-scan thermally polarized ¹³C signal from 4 M sodium [1-¹³C] acetate using similar acquisition parameters. Enhancement is ε-9000 and polarization is about P(¹³C) ˜1%.

All experiments were performed with the solution containing fluorinated catalyst, co-ligand (phenyl trifluoromethyl sulfoxide) and [1-¹³C] Pyruvate in 0.6 mL CD₃OD. The ratio and concentration can be further adjusted to attain better enhancement and polarization. The results were obtained for 8 mM fluorinated catalyst, 16 mM phenyl trifluoromethyl sulfoxide (PTFSO) and 30 mM [1-¹³C] Pyruvate in 0.6 mL CD₃OD. The experiments were performed at room temperature, ˜100 scc/m p-H₂ flow rate and 96 PSI p-H₂ overpressure. The parahydrogen used in this example came from a low-cost 50% p-H₂ generator. Each experiment, the p-H₂ bubbling was applied for ˜1 min, the sample was quickly transferred to the 1 T NMR spectrometer for detection and the sample was then returned to the mu-metal shield to continue p-H₂ bubbling for the next experiment. The ¹³C signal enhancement was computed by comparing HP signal area-undercurve (AUC) to external ¹³C signal thermal signal reference (4M sodium [1-¹³C]acetate) using Eq. 1:

$$ε ( {}^{13} C ) = \frac{{S}_{HP}}{{S}_{REF}} \cdot \frac{{C}_{REF}}{{C}_{HP}} \cdot \frac{{A}_{REF}}{{A}_{HP}} , \qquad ( 1 )$$

where S_{HP} and S_{REF} are ¹³C signals from HP [1-¹³C] pyruvate and thermal signal reference [1-¹³C]acetate, C_{REF} and C_{HP} are concentrations of thermal signal reference [1-¹³C]acetate (4 M) and of HP [1-¹³C]pyruvate, respectively, and A_{REF} and A_{HP} are effective cross-sections of the NMR tubes for the thermal signal reference [1-¹³C]acetate and HP [1-¹³C]pyruvate samples.

### Example 6

This example illustrates a method of hyperpolarizing a [1-¹³C]pyruvate in accordance with an aspect of the invention, as shown in Scheme 2.

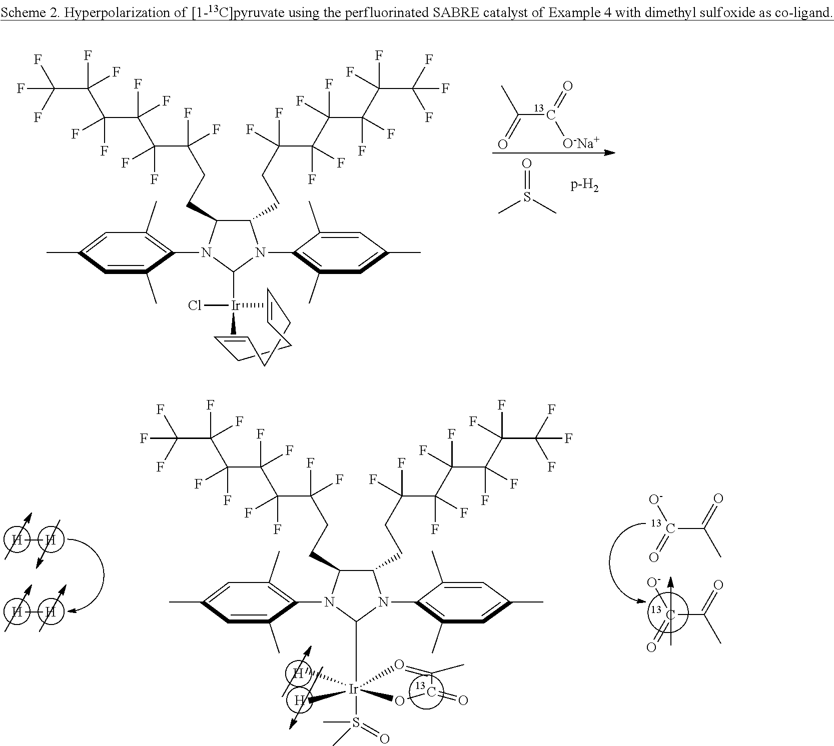

In the reaction scheme above, hyperpolarization of [1-¹³C]pyruvate was performed using SABRE in SHield Enables Alignment Transfer to Heteronuclei (SABRE-SHEATH) (Theis et al., J. Am. Chem. Soc., 137, 1404-1407 (2015) and Truong et al., J. Phys. Chem. C, 119, 8786-8797 (2015)) tailored for the ¹³C nucleus (Barskiy et al., ChemPhysChem, 18, 1493-1498 (2017)) using the co-ligand approach developed by Duckett and co-workers (Iali et al., Angew. Chemie —Int. Ed., 58, 10271-10275 (2019)). Sodium [1-¹³C]-pyruvate and deuterated methanol-d₄ solvent were purchased from Sigma-Aldrich and used without any further purification. The [IrCl(COD)(F-IMes)] SABRE catalyst used for this Example was prepared according to Example 4. The active catalyst used herein was prepared with a fixed ratio of substrate [1-¹³C]pyruvate, Ir(F-IMes) SABRE catalyst of Example 4, and co-ligand dimethyl sulfoxide (DMSO), in 0.6 mL of methanol-d₄ in a 5 mm NMR tube.

Parahydrogen enriched to about 70 to 95% was used and directed via PTFE tubing to a mass flow controller (MFC, Sierra Instruments SmartTrak 100 series) set between 50 to 120 scc/m into a medium wall 5 mm NMR tube (Norell) to allow bubbling through the sample. The entire pH₂line was pressurized values between 50 and 110 psi.

The polarization transfer magnetic field was established as follows. Magnetic fields near or below ˜1 μT were achieved with an apparatus consisting of a solenoid coil placed inside a three-layered mu-metal shield (6 in. ID & 15 in. in length, part number ZG-206, Magnetic Shield Corp., Bensenville, IL). The magnetic field was created using a custom-built solenoid coil and a triple independent channel DC power supply (KEITHLEY 2231A-30-3). The solenoid had a 41 mm diameter (40 mm core, 20 cm long windings with 220 turns AWG20 (0.9 mm) Cu wire and with 220Ω resistor in series. The solenoid coil was driven with a variable-resistance decade box in series to provide finer control of the internal magnetic field inside the shield. Typical values of the field within the shield were between ±1.2μT, with SABRE SHEATH experiments typically between −0.7 μT and +0.8 μT in the sample region.

MR experiments were performed using an 80 MHz Magritek Spinsolve benchtop NMR spectrometer. The following acquisition parameters were used:
spectra width (SW)=5 kHz;
dwell time (DT)=150 μs;
number of scans (ns)=1,
receiver gain=16;
excitation pulse angle (a)=90°;
¹³C resonance frequency=20.25232790 MHz.

All ¹³C NMR spectra were taken with ¹H decoupling turned off throughout the duration of the experiment. Time required to manually transfer the sample from the shield region to the magnet for low-field NMR acquisition was usually <5 s.

The hyperpolarization transfer from p-H₂-derived iridium hydrides to the ¹³C nuclear spin of [1-¹³C]pyruvate was attained by performing SABRE in sub-microtesla magnetic fields using SABRE in SHield Enables Alignment Transfer to Heteronuclei (SABRE-SHEATH) using a solution mixture of [IrCl(H)₂(DMSO)₂(F-IMes)], [1-¹³C]pyruvate, co-ligand (dimethyl sulfoxide) and p-H₂ in 0.5 mL deuterated or non-deuterated methanol.

The ¹³C signal enhancement was computed by comparing HP signal area-undercurve (AUC) to external ¹³C signal thermal signal reference (4M sodium [1-¹³C]acetate) using Eq.:

$$ε ( {}^{13} C ) = \frac{{S}_{HP}}{{S}_{REF}} \cdot \frac{{C}_{REF}}{{C}_{HP}} \cdot \frac{{A}_{REF}}{{A}_{HP}} , \qquad ( 1 )$$

where S_{HP} and S_{REF} are ¹³C signals from HP [1-¹³C] pyruvate and thermal signal reference [1-¹³C]acetate, C_{REF} and C_{HP} are concentrations of thermal signal reference [1-¹³C]acetate (4 M) and of HP [1-¹³C]pyruvate, respectively, and A_{REF} and A_{HP} are effective cross-sections of the NMR tubes for the thermal signal reference [1-¹³C]acetate and HP [1-¹³C]pyruvate samples. The percentage of ¹³C polarization (% P¹³c) was computed by multiplying the signal enhancement (ε¹³c) by thermal ¹³C nuclear spin polarization at 1.88 T (1.5681*10⁻⁴%) in accordance with Equation S2: (% P¹³c), =ε¹³c 1.56181*10⁻⁶ *100.

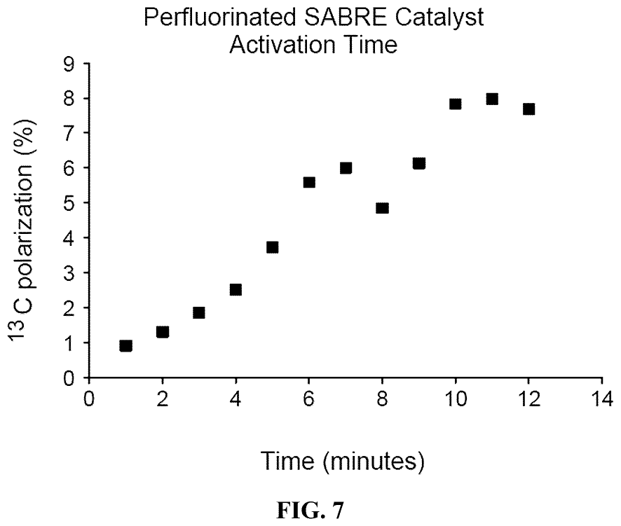

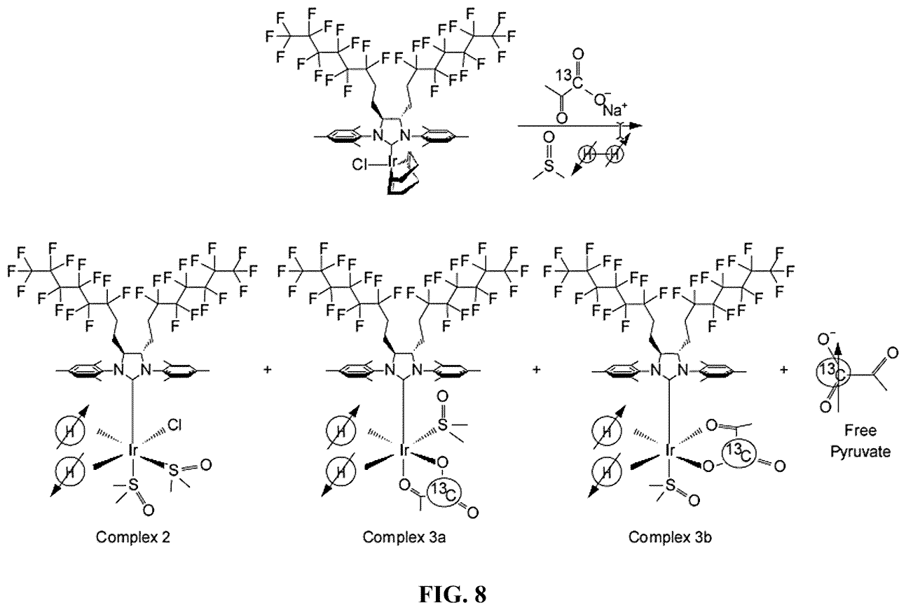

The fluorinated SABRE catalyst activation took less than 15 minutes, with the ¹³C polarization percentage shown in FIG. 7, and is performed by bubbling ˜95% p-H₂ at a flow rate of 90 standard cubic centimeters per minute (scc/m) at 8 atm p-H₂ partial pressure, which leads to the formation of Complex 2, Complex 3a, Complex 3b, and pyruvate, as depicted in FIG. 8, in accord with the notation introduced by Duckett and co-workers (Iali et al., Angew. Chemie —Int. Ed., 58, 10271-10275 (2019)). Without wishing to be bound by any particular theory, it is believed that Complex 3B is the primary SABRE-active species.

### Example 7

This example demonstrates the effects on hyperpolarization of [1-¹³C]pyruvate, exhibited by changes in parahydrogen pressure and flow rate, as well as the effect of magnetic transfer field, temperature, and concentration of the fluorinated catalyst and DMSO. In addition, the relaxation dynamics of the [1-¹³C]pyruvate were also studied.

Hyperpolarization of [1-¹³C]pyruvate was repeated using SABRE in SHield Enables Alignment Transfer to Heteronuclei (SABRE-SHEATH), as described in Example 6 above, and the effects of parahydrogen pressure and flow rate, as well as the effect of magnetic transfer field, temperature, and concentration of the fluorinated catalyst and DMSO, were studied.

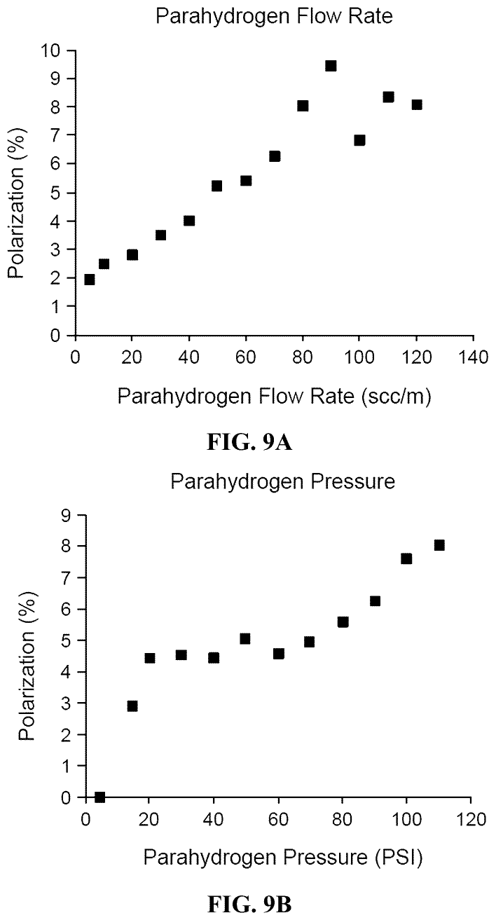

p-H₂ parameters such as the pressure and flow rate were evaluated and the polarization percentage results are set forth in FIGS. 9A and 9B. The NMR samples contained in 30 mM sodium [1-¹³C]pyruvate, 2.6 mM fluorinated SABRE catalyst, and 40 mM dimethyl sulfoxide (DMSO), the mixing field was at 0.4 μT and temperature at 0° C. As is apparent from the results set forth in FIGS. 9A and 9B, as the parahydrogen flow rate and pressure increased, the polarization percentage also increased.

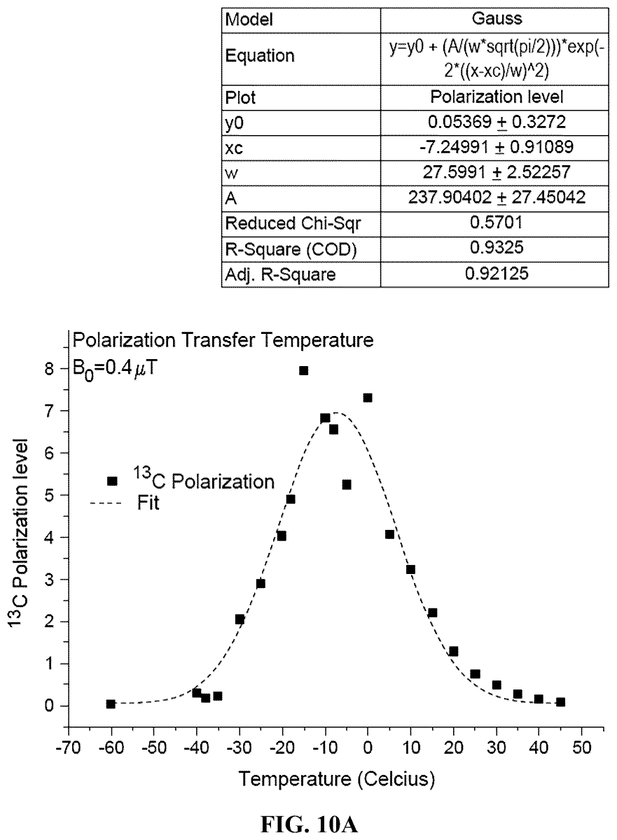

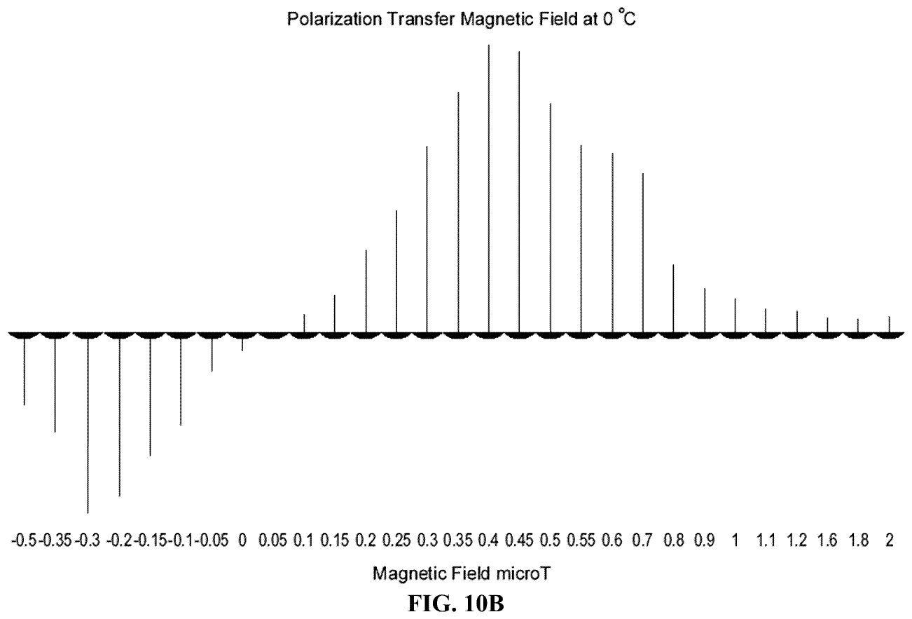

The temperature and magnetic field in the micro Tesla regime were evaluated and the 13C polarization level and polarization transfer magnetic field at 0° C. are set forth in FIGS. 10A and 10B, respectively. The NMR samples contained in 30 mM sodium [1-¹³C]pyruvate, 2.6 mM fluorinated SABRE catalyst, and 40 mM dimethyl sulfoxide (DMSO), p-H₂ flow and pressure 70 scc/m and 100 PSI. As is apparent from the results set forth in FIGS. 10A and 10B, the best polarization transfer occurs at temperatures between −20° C. and 5° C. and a mixing field between 0.3 μT and 0.5 μT. The optimum temperature is −7.24° C. and the optimum mixing field is 0.4 μT.

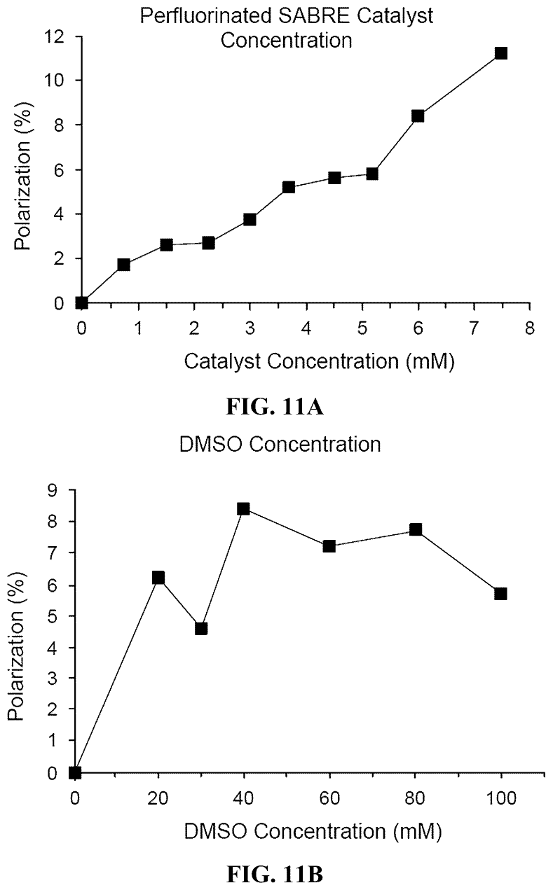

The perfluorinated SABRE catalyst and DMSO concentrations were evaluated at a temperature of 0° C., a magnetic transfer field of 0.4 μT, a p-H₂ flow of 90 scc/m, and a p-H₂ pressure of 110 PSI, and the polarization percentages are set forth in FIGS. 11A and 11B. As is apparent from the results set forth in FIG. 11A, the polarization percentage increases as the perfluorinated SABRE catalyst concentration increases. However, the polarization percentage remains relatively consistent at concentrations above 20 mM.

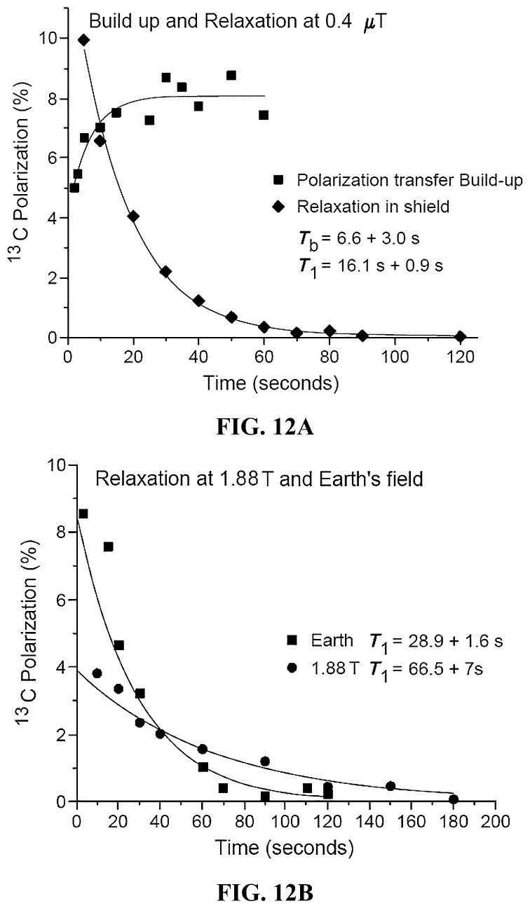

As is apparent from the results set forth in FIGS. 12A and 12B, the relaxation dynamics of [1-¹³C]-pyruvate show that the total P_{13C} (bound+free) build-up time (T_{b}=6.6±3.0 s) is substantially shorter than the corresponding T₁ value of 16.1±0.9 s, which allows to reach P_{13C} levels up to 13.48%. In addition, relaxation dynamics at earth field and 1.8 T are about the same as a non-fluorinated SABRE catalyst T₁=28.9±1.6 s and 66.5±7 s, respectively.

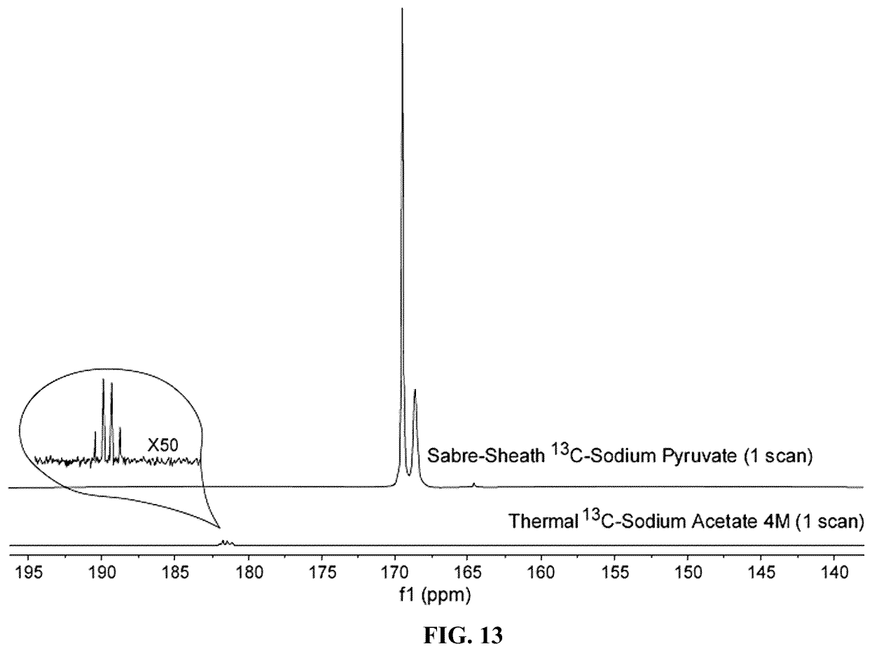

The simultaneous exchange of p-H₂ and [1-¹³C]pyruvate on activated Ir(F-IMes) catalyst leads to buildup of ¹³C hyperpolarization. In that respect, FIG. 13 shows a representative spectrum of ¹³C-hyperpolarized [1-¹³C]-pyruvate with signal enhancement ε of ˜86500 fold, corresponding to P_{13C} of ˜13.48% obtained via comparison of the NMR signal intensity with a reference sample. The NMR samples contained in 20 mM sodium [1-¹³C]pyruvate, 2.6 mM fluorinated SABRE catalyst, and 40 mM dimethyl sulfoxide (DMSO), the mixing field was at 0.4 μT and temperature at 0° C. with a parahydrogen pressure and flow at 110 PSI and 90 scc/m, respectively.

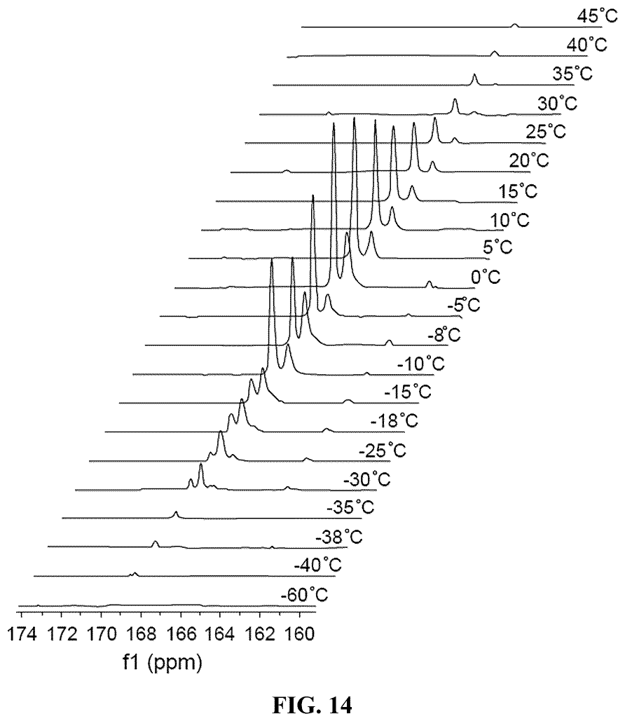

Temperature has a profound effect on the exchange rates of [1-¹³C]pyruvate on Complex 3b of FIG. 7. In recent work, Adelabu et al. (ChemPhysChem, 23, e202100839 (2022)) showed that the monotonic disappearance of free HP resonance at low temperatures happened due to the slow exchange rate of Complex 3b into the free state. At room temperature (e.g., 22° C.), the exchange of [1-¹³C]pyruvate with the polarization transfer complex was faster, leading to hyperpolarization of both free and bound 3b species in the expected pyruvate: pre-catalyst ratio. In order to rapidly release the HP pyruvate from 3b, the HP solution was rapidly warmed up then the sample was inserted in the NMR detector (TomHon et al., J Am. Chem. Soc., 144(1) 282˜287 (2022)). FIG. 14 shows a variable temperature SABRE-SHEATH experiment using the saturated perfluorinated SABRE catalyst of Example 4. The NMR samples (in deuterated methanol) contained in 25 mM sodium [1-¹³C]pyruvate, 6 mM perfluorinated SABRE catalyst, and 47 mM dimethyl sulfoxide (DMSO), wherein the mixing field was at 0.4 μT, and the parahydrogen pressure and flow rate were set at 110 PSI and 90 scc/m, respectively.

As demonstrated by FIG. 14, the exchange rate of Complex 3b into the free state is faster even at low temperature, such as −10° C., where most of the HP [1-¹³C]pyruvate is in a free state. By decreasing the temperature of ¹³C SABRE-SHEATH to 0° C., P_{13C} maximum is achieved. The exchange remains fast enough to build-up preferentially the “free” HP [1-¹³C]pyruvate over Complex 3b.

### Example 8

This example illustrates an exemplary method for isolating hyperpolarized sodium [1-¹³C]pyruvate, which includes extraction and filtration.

Hyperpolarization of sodium [1-¹³C]pyruvate was performed using SABRE in SHield Enables Alignment Transfer to Heteronuclei (SABRE-SHEATH) tailored for the ¹³C nucleus using dimethyl sulfoxide (DMSO) as a co-ligand, [IrCl(COD)(F-IMes)] SABRE catalyst, and parahydrogen enriched to about 70 to 95% in deuterated methanol-d₄ solvent, as described in Examples 6 and 7. The SABRE samples were prepared in 0.5 mL CD₃OD, using 30 mM sodium [1-¹³C]pyruvate, 2.6 mM perfluorinated SABRE catalyst of Example 4, and 35 mM dimethyl sulfoxide (DMSO). The parahydrogen flow rate was established at 90 scc/m and pressurized to 8 bars, the mixing field was 0.4 μT, and the temperature was 0° C.

After the hyperpolarization procedure was completed, the sample was rapidly removed from the 0.40 μT field, depressurized, and 20% in volume (125 P L) of heavy water (D₂O) was added to the solution to precipitate the perfluorinated SABRE catalyst. The resulting mixture was transferred into a 1 mL plastic syringe mounted to a Luer-locked filter (Waters Oasis Prime HLB Plus Light Cartridge (Part Number: 186008866), and the aqueous solution was guided through the filter into a 5 mm NMR tube, already located into the adjacent 1.8 T benchtop NMR spectrometer. The whole procedure took about 1.15 to 1.30 minutes and no HP ¹³C signal was observed in any sample. The relaxation study presented above indicates that [1-¹³C]-T₁ relaxation time of pyruvate at earth field was substantially shorter (e.g., [1-¹³C]-T₁=28.9±1.6 s at Earth's field). Automation and faster solution transfer should allow the observation of HP [1-¹³C]-pyruvate signal in aqueous solutions.

### Example 9

This example illustrates an exemplary method for isolating hyperpolarized sodium [1-¹³C]pyruvate, which includes extraction by precipitation with organic solvent.

Precipitation and redissolution of HP [1-13C]pyruvate, which takes place in the same NMR tube where hyperpolarization, was performed (Schmidt et al., ACS Sensors, 7(11), 3430-3439 (2022)). The SABRE samples were prepared in CD₃OD, using between 20 and 30 mM sodium [1-¹³C]pyruvate, 7.5 mM fluorinated SABRE catalyst, and 50 mM dimethyl sulfoxide (DMSO), as described in Examples 6 and 7. 100 μL of this solution was transferred into 5 mm NMR tube and exposed to the SABRE-SHEATH hyperpolarization conditions with the same set-up and optimum conditions described in Examples 6 and 7. The solution was located inside a 3-layer mu-metal of 3″ I.D. and 9″ depth to shield external magnetic fields, combined with a custom-made solenoid to generate a static magnetic field Bo of 0.4 μT. The NMR tubes were pressurized (110 psi, i.e., approximately 8 bar total pressure) with p-H₂ bubbling through the solution at a flow of 90 scc/m to activate the catalyst and to ¹³C-hyperpolarize the sodium [1-¹³C]pyruvate solution. Activation of the catalyst took place for 15 min at ambient temperature and magnetic field. For polarization build-up, the sample was placed in the static magnetic field (typically about 0.4 μT) and a water bath to regulate the reaction temperature at 0° C. After polarization, the NMR tube was rapidly transferred inside the NMR spectrometer at 1.8 T and kept at room temperature. The precipitation of pyruvate is performed after depressurization by adding 400 μL of ethyl acetate (EtOAc) to the HP solution and redissolved by adding 300 μL heavy water (D₂O) to reconstitute the pyruvate in water.

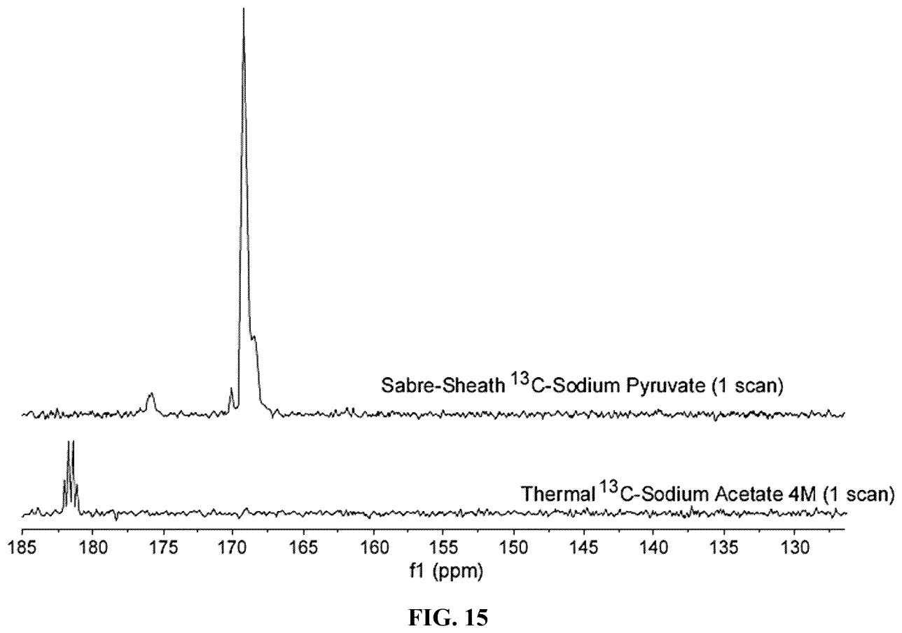

The NMR spectrum was acquired immediately after reconstitution in water using a 1.8 T benchtop NMR, and the results are set forth in the top spectrum of FIG. 15. In addition, the bottom spectrum of FIG. 15 shows a single-scan thermally polarized ¹³C signal from 4 M sodium [1-¹³C] acetate using similar acquisition parameters.

In addition, the ¹³C-pyruvate concentrations were determined by LC-MS using a calibration curve for naturally occurring isotopic pyruvate. The pyruvate aqueous samples were further analyzed by ICP-MS (inductively coupled plasma Multi-Element Scan) for Iridium elemental content after the reconstitution SABRE-SHEATH methodology. The Iridium content was determined to be only about 150 ppb to about 300 ppm.

### Example 10

This example illustrates a method of the synthesis of 2,2,2-trifluoro-N-(4-iodo-2,6-dimethylphenyl)acetamide:

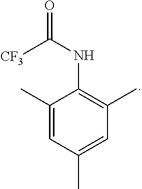

To a stirring solution of 2,6-diisopropylaniline (4.92 mL, 40.00 mmol, 1.0 equiv) in diethyl ether (50 mL) were added iodine (11.17 g, 44.00 mmol, 1.1 equiv) and a saturated sodium bicarbonate solution (30 mL). The solution was stirred at room temperature for 3 hours and gas evolution was observed. Excess iodine was destroyed by addition of sodium thiosulfate (1.33 g, 8.40 mmol). The phases were separated, and the aqueous phase was further extracted with diethyl ether (2×20 mL). The combined organic phases were washed with water (200 mL) and saturated solution of sodium thiosulfate. The organic phase was evaporated to dryness in vacuo to yield the product as a brown oil which slowly became solid under vacuum.

Hexane was added to dissolve product and the solution is filtered with Celite and evaporated and dried under vacuum. No further purification was necessary. ¹H NMR (400 MHz, CDCl₃): δ=7.14 (s, 2H), 4.45 (s, 2H), 2.04 (s, 6H).

### Example 11

This example illustrates a method of synthesis of N-(2,6-dimethyl-4-(3,3,4,4,5,5,6,6,7,7,8,8,9,9,10,10,10-tridecafluorooct-1-en-1-yl)phenyl)-2,2,2-trifluoroacetamide:

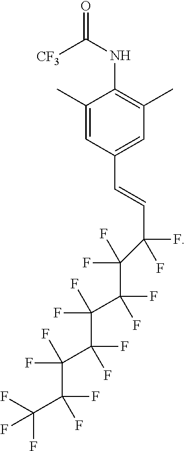 [SMILES: Cc1cc(/C=C/C(F)(F)C(F)(F)C(F)(F)C(F)(F)C(F)(F)C(F)(F)C(F)(F)C(F)(F)F)cc(C)c1NC(=O)C(F)(F)F]

In a glove box, palladium(II) acetate (0.16 g, 0.051 equiv, 0.71 mmol), sodium acetate (1.72 g, 1.50 equiv, 21.0 mmol), tricyclohexylphosphane (660 mg, 0.168 equiv, 2.35 mmol), tetrabutylammonium bromide (30 mg, 0.0067 equiv, 93 μmol) and 2,2,2-trifluoro-N-(4-iodo-2,6-dimethylphenyl)acetamide (4 g, 0.8 equiv, 0.01 mol) were added to a Schlenk flask with 10 mL of dimethyl formamide. The Schlenk flask was warmed to 90° C., then 3,3,4,4,5,5,6,6,7,7,8,8,9,9,10,10,10-tridecafluorooct-1-ene (4.84 g, 1 equiv, 14.0 mmol) was added to the reaction mixture and further heated to 120° C. The solution was stirred for 19 hours. After cooling, the mixture was filtered through Celite and washed with diethyl ether (50 mL). Water (50 mL) and diethyl ether (30 mL) were added, the organic phase was separated, and aqueous phase was extracted with Et₂O (3×15 mL). The combined organic layers were dried over anhydrous MgSO₄ and concentrated under reduced pressure to afford the crude product. Purification by column chromatography (hexane/DCM 4:1) gave fluoroamide N-(2,6-dimethyl-4-(3,3,4,4,5,5,6,6,7,7,8,8,9,9,10,10,10-tridecafluorooct-1-en-1-yl)phenyl)-2,2,2-trifluoroacetamide, which was further crystallized (5 g, 70%, white needles).

### Example 12

This example illustrates a synthesis of N-(2,6-dimethyl-4-(3,3,4,4,5,5,6,6,7,7,8,8,9,9,10,10,10-tridecafluorooctyl)phenyl)-2,2,2-trifluoroacetamide:

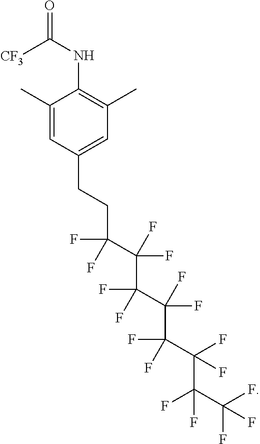 [SMILES: Cc1cc(CCC(F)(F)C(F)(F)C(F)(F)C(F)(F)C(F)(F)C(F)(F)C(F)(F)C(F)(F)F)cc(C)c1NC(=O)C(F)(F)F]

To a solution of N-(2,6-dimethyl-4-(3,3,4,4,5,5,6,6,7,7,8,8,9,9,10,10,10-tridecafluorooct-1-en-1-yl)phenyl)-2,2,2-trifluoroacetamide (0.40 g, 0.70 mmol) in ethyl acetate (10 mL) in a glass autoclave, 10% Pd/C (0.08 g, 0.07 mmol) was added. The autoclave was evacuated, filled with hydrogen gas to 500 kPa and stirred for 5 hours at room temperature. The mixture was filtered through Celite and washed with EtOAc (30 mL). The solvent was removed on vacuum rotary evaporator to afford N-(2,6-dimethyl-4-(3,3,4,4,5,5,6,6,7,7,8,8,9,9,10,10,10-tridecafluorooctyl)phenyl)-2,2,2-trifluoroacetamide.

### Example 13

This example illustrates a synthesis of 2,6-dimethyl-4-(3,3,4,4,5,5,6,6,7,7,8,8,9,9,10,10,10-tridecafluorooctyl)aniline:

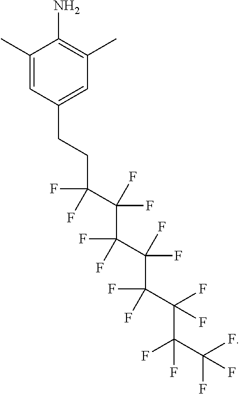 [SMILES: Cc1cc(CCC(F)(F)C(F)(F)C(F)(F)C(F)(F)C(F)(F)C(F)(F)C(F)(F)C(F)(F)F)cc(C)c1N]

To a solution of N-(2,6-dimethyl-4-(3,3,4,4,5,5,6,6,7,7,8,8,9,9,10,10,10-tridecafluorooctyl)phenyl)-2,2,2-trifluoroacetamide (0.14 g, 250 μmol) in n-butanol (1.5 mL), sodium hydroxide (0.10 g, 250 mmol) was added and the reaction mixture was heated to 110° C. for 21 hours. After cooling to room temperature, water (4 mL) and ethyl acetate (4 mL) were added and the organic phase was separated, washed with 1M solution of HCl (4 mL), saturated solution of NaHCO₃(4 mL), and brine (4 mL). The aqueous phase was neutralized and extracted with ethyl acetate (3 times ×4 mL). The organic layers were combined and dried over anhydrous magnesium sulfate and concentrated under reduced pressure to afford 2,6-dimethyl-4-(3,3,4,4,5,5,6,6,7,7,8,8,9,9,10,10,10-tridecafluorooctyl)aniline (0.50 g, 92%, brown crystals).

### Example 14

This example illustrates a synthesis of N,N′-Bis[2,6-dimethyl-4-(3,3,4,4,5,5,6,6,7,7,8,8,9,9,10,10,10-tridecafluorooctyl)phenyl]ethane-1,2-diimine:

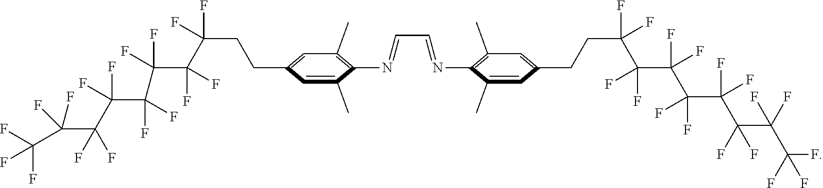

To the solution of 2,6-dimethyl-4-(3,3,4,4,5,5,6,6,7,7,8,8,9,9,10,10,10-tridecafluorooctyl)aniline (0.5 g, 1 mmol) in ethanol (5 mL), 40% aqueous solution of glyoxal (0.1 mL, 1 mmol) and catalytic amount of formic acid (few drops) were added. The reaction mixture was stirred for 20 hours at room temperature, during which yellow precipitate was formed. The solid was filtered and washed with cold ethanol (3×5 mL) to give clean N,N-bis(2,6-dimethyl-4-(3,3,4,4,5,5,6,6,7,7,8,8,9,9,10,10,10-tridecafluorooctyl)phenyl)ethane-1,2-diimine (0.2 g, 20%, yellow powder).

### Example 15

This example illustrates a synthesis of 1,3-bis(2,6-dimethyl-4-(3,3,4,4,5,5,6,6,7,7,8,8,9,9,10,10,10-tridecafluorooctyl)phenyl)-1H-imidazol-3-ium-2-ide, chloride salt:

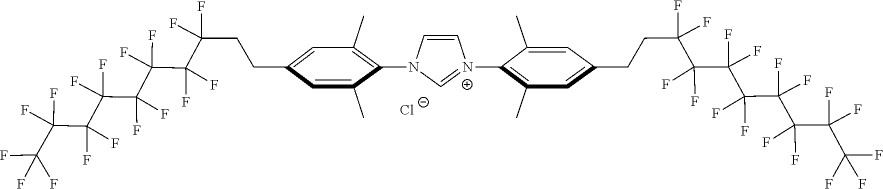

A flask was charged with N,N-bis(2,6-dimethyl-4-(3,3,4,4,5,5,6,6,7,7,8,8,9,10,10,10-tridecafluorooctyl)phenyl)ethane-1,2-diimine (2.40 g, 2.08 mmol) and tetrahydrofuran (80 mL). The mixture was cooled to 0° C. and suspension of paraformaldehyde (262 mg, 2.91 mmol) and conc. HCl (112 mg, 3.12 mmol) in dioxane (0.78 mL) was slowly added. The mixture was heated to reflux overnight. Purification was carried out by column chromatography. ¹H NMR (400 MHz, CDCl₃) δ 2.07, 2.55, 2.92, 7.37, 8.28, 9.65. MS (ESI) m/z (%): 1168.8 [M-TFA]⁺(100).

### Example 16

This example illustrates a synthesis of a fluorinated SABRE catalyst in accordance with an aspect of the invention.

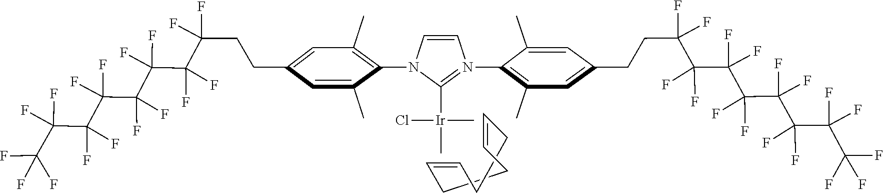

Potassium tert-butoxide (2.5 eq.) was added to a stirred solution of 1,3-bis(2,6-dimethyl-4-(3,3,4,4,5,5,6,6,7,7,8,8,9,9,10,10,10-tridecafluorooctyl)phenyl)-1H-imidazol-3-ium-2-ide, chloride (2.2 equiv) in tetrahydrofuran at room temperature in a glove box. The resulting suspension was stirred for 30 min. A solution of [Ir(COD)Cl]⁺(1.0 eq.) was added and the resulting solution was stirred at room temperature for 2 hours. The solvent was removed under reduced pressure to give the crude product and dried overnight under vacuum. This sample was purified with flash chromatography DCM/hexane (4:1) to obtain the fluorinated SABRE catalyst. MS (ESI) m/z (%): 1469 [M-Cl]⁺ (100).

### Example 17

This example illustrates the hyperpolarization of sodium pyruvate using the SABRE catalyst in accordance with an aspect of the invention, as shown in Scheme 3.

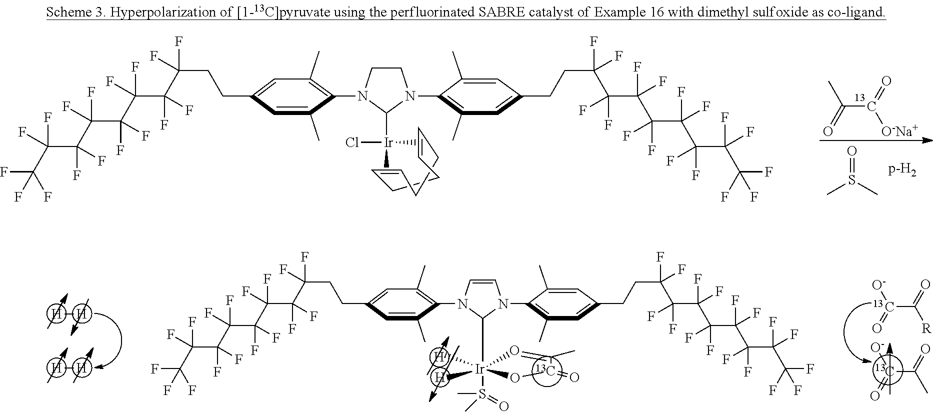

In the reaction scheme above, the efficient hyperpolarization transfer from p-H₂-derived hydrides to the ¹³C nuclear spin of [1-¹³C]pyruvate was attained by performing SABRE in sub-microtesla magnetic fields using SABRE in SHield Enables Alignment Transfer to Heteronuclei (SABRE-SHEATH) using a solution mixture of [IrCl(H)₂(DMSO)₂(F-IMes)], [1-¹³C]pyruvate) and p-H₂ in deuterated methanol.

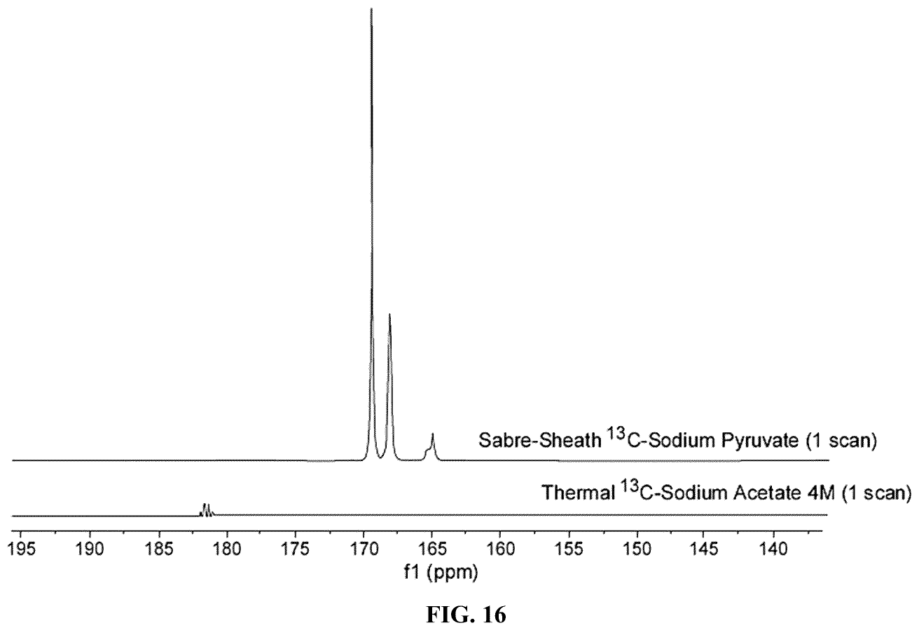

The SABRE samples were prepared in CD₃OD, using 40 mM sodium [1-¹³C]pyruvate, 6.6 mM perfluorinated SABRE catalyst of Example 16, and 50 mM dimethyl sulfoxide (DMSO), as described in Examples 6 and 7. The SABRE samples were exposed to the SABRE-SHEATH hyperpolarization conditions with the same set-up and optimum conditions described in Examples 6 and 7. The NMR tubes were pressurized (110 psi, i.e., approximately 8 bar total pressure) with p-H₂ bubbling through the solution at a flow of 90 scc/m to activate the catalyst and to ¹³C-hyperpolarize the sodium [1-¹³C]pyruvate solution. Activation of the catalyst took place for about 15 minutes at ambient temperature and magnetic field. For polarization build-up, the sample was placed in the magnetic field (typically about 0.4 μT) and a water bath at 5° C. to regulate the reaction temperature. The spectrum was acquired immediately following manual sample transfer to a 1.8 T benchtop NMR after 5 seconds, and the results are set forth in the top spectrum of FIG. 16. In addition, the bottom spectrum of FIG. 16 shows a single-scan thermally polarized ¹³C signal from 4 M sodium [1-¹³C] acetate using similar acquisition parameters. As is apparent from the results set forth in FIG. 16, the signal enhancement is ε-16900 and polarization is about P(¹³C) 2.17%.

### Example 18

This example illustrates a method of synthesis of N-(2,6-dimethyl-4-(3,3,4,4,5,5,6,6,7,7,8,8,8-tridecafluorooct-1-en-1-yl)phenyl)-2,2,2-trifluoroacetamide:

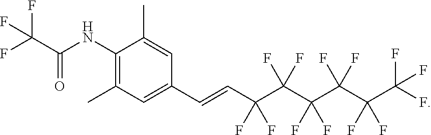 [SMILES: Cc1cc(/C=C/C(F)(F)C(F)(F)C(F)(F)C(F)(F)C(F)(F)C(F)(F)F)cc(C)c1NC(=O)C(F)(F)F]

In a glove box, palladium(II) acetate (0.16 g, 0.051 equiv, 0.71 mmol), sodium acetate (1.72 g, 1.50 equiv, 21.0 mmol), tricyclohexylphosphane (660 mg, 0.168 equiv, 2.35 mmol), tetrabutylammonium bromide (30 mg, 0.0067 equiv, 93 μmol) and 2,2,2-trifluoro-N-(4-iodo-2,6-dimethylphenyl)acetamide (4 g, 0.8 equiv, 0.01 mol) were added to a Schlenk flask with 10 mL of dimethyl formamide. The Schlenk flask was warmed to 90° C., then 3,3,4,4,5,5,6,6,7,7,8,8,8-tridecafluorooct-1-ene (4.84 g, 1 equiv, 14.0 mmol) was added to the reaction mixture and further heated to 120° C. The solution was stirred for 19 hours. After cooling, the mixture was filtered through Celite and washed with diethyl ether (50 mL). Water (50 mL) and diethyl ether (30 mL) were added, the organic phase was separated, and aqueous phase was extracted with Et₂O (3×15 mL). The combined organic layers were dried over anhydrous MgSO₄ and concentrated under reduced pressure to afford the crude product. Purification by column chromatography (hexane/DCM 4:1) gave fluoroamide N-(2,6-dimethyl-4-(3,3,4,4,5,5,6,6,7,7,8,8,8-tridecafluorooct-1-en-1-yl)phenyl)-2,2,2-trifluoroacetamide, which was further crystallized (5 g, 70%, white needles).

### Example 19

This example illustrates a synthesis of 2,6-dimethyl-4-(3,3,4,4,5,5,6,6,7,7,8,8,8-tridecafluorooctyl)aniline:

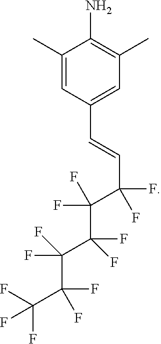 [SMILES: Cc1cc(/C=C/C(F)(F)C(F)(F)C(F)(F)C(F)(F)C(F)(F)C(F)(F)F)cc(C)c1N]

To a solution of N-(2,6-dimethyl-4-(3,3,4,4,5,5,6,6,7,7,8,8,8-tridecafluorooct-1-en-1-yl)phenyl)-2,2,2-trifluoroacetamide (0.14 g, 250 μmol) in n-butanol (1.5 mL), sodium hydroxide (0.10 g, 250 mmol) was added and the reaction mixture was heated to 110° C. for 21 hours. After cooling to room temperature, water (4 mL) and ethyl acetate (4 mL) were added and the organic phase was separated, washed with 1M solution of HCl (4 mL), saturated solution of NaHCO₃(4 mL), and brine (4 mL). The aqueous phase was neutralized and extracted with ethyl acetate (3 times×4 mL). The organic layers were combined and dried over anhydrous magnesium sulfate and concentrated under reduced pressure to afford 2,6-dimethyl-4-(3,3,4,4,5,5,6,6,7,7,8,8,8-tridecafluorooctyl)aniline (0.50 g, 92%, brown crystals).

### Example 20

This example illustrates a synthesis of N,N′-Bis[2,6-dimethyl-4-(3,3,4,4,5,5,6,6,7,7,8,8,8-tridecafluorooct-1-en-1-yl)phenyl]ethane-1,2-diimine:

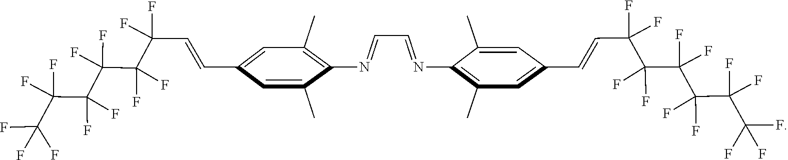

To the solution of 2,6-dimethyl-4-(3,3,4,4,5,5,6,6,7,7,8,8,8-tridecafluorooct-1-en-1-yl)aniline (0.5 g, 1 mmol) in ethanol (5 mL), 40% aqueous solution of glyoxal (0.1 mL, 1 mmol) and catalytic amount of formic acid (few drops) were added. The reaction mixture was stirred for 20 hours at room temperature, during which yellow precipitate was formed. The solid was filtered and washed with cold ethanol (3×5 mL) to give clean N,N-bis(2,6-dimethyl-4-(3,3,4,4,5,5,6,6,7,7,8,8,8-tridecafluorooctyl)phenyl)ethane-1,2-diimine (0.2 g, 20%, yellow powder).

### Example 21

This example illustrates a synthesis of 1,3-bis(2,6-dimethyl-4-(3,3,4,4,5,5,6,6,7,7,8,8,8-tridecafluorooct-1-en-1-yl)-1H-imidazol-3-ium-2-ide, chloride salt:

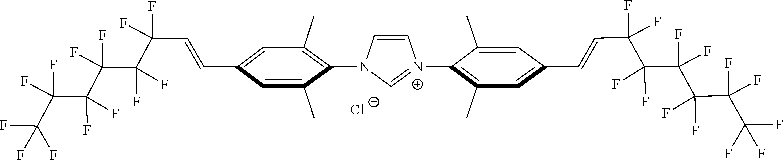

A flask was charged with N,N-bis(2,6-dimethyl-4-(3,3,4,4,5,5,6,6,7,7,8,8,8-tridecafluorooct-1-en-1-yl)phenyl)ethane-1,2-diimine (2.40 g, 2.08 mmol) and tetrahydrofuran (80 mL). The mixture was cooled to 0° C. and suspension of paraformaldehyde (262 mg, 2.91 mmol) and conc. HCl (112 mg, 3.12 mmol) in dioxane (0.78 mL) was slowly added. The mixture was heated to reflux overnight. Purification was carried out by column chromatography. ¹H NMR (400 MHz, CDCl₃) δ 2.27, 6.71, 7.32, 7.36, 7.67, 8.16, 9.59 ppm. ¹⁹F NMR (400 MHz, CDCl₃) δ-82.43, −112.52, −122. 59, −123.89, −124.17, −127.33 ppm. MS (ESI) m/z (%): 964.8 [M+H]⁺ (100).

### Example 22

This example illustrates a synthesis of a fluorinated SABRE catalyst in accordance with an aspect of the invention.

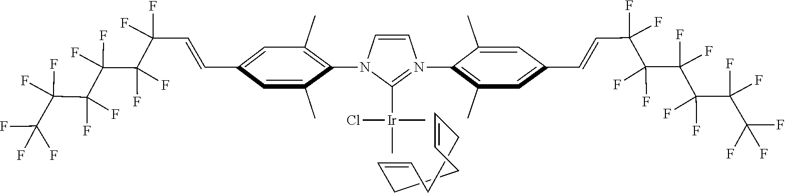

Potassium tert-butoxide (2.5 eq.) was added to a stirred solution of 1,3-bis(2,6-dimethyl-4-(3,3,4,4,5,5,6,6,7,7,8,8,8-tridecafluorooct-1-en-1-yl)phenyl)-1H-imidazol-3-ium-2-ide, chloride (2.2 equiv) in tetrahydrofuran at room temperature in a glove box. The resulting suspension was stirred for 30 min. A solution of [Ir(COD)Cl]₂ (1.0 eq.) was added and the resulting solution was stirred at room temperature for 2 hours. The solvent was removed under reduced pressure to give the crude product and dried overnight under vacuum. This sample was purified with flash chromatography DCM/hexane (4:1) to obtain the fluorinated SABRE catalyst. MS (ESI) m/z (%): 1266 [M-Cl]⁺ (100).

### Example 23

This example illustrates the hyperpolarization of sodium pyruvate using the SABRE catalyst in accordance with an aspect of the invention, as shown in Scheme 4.

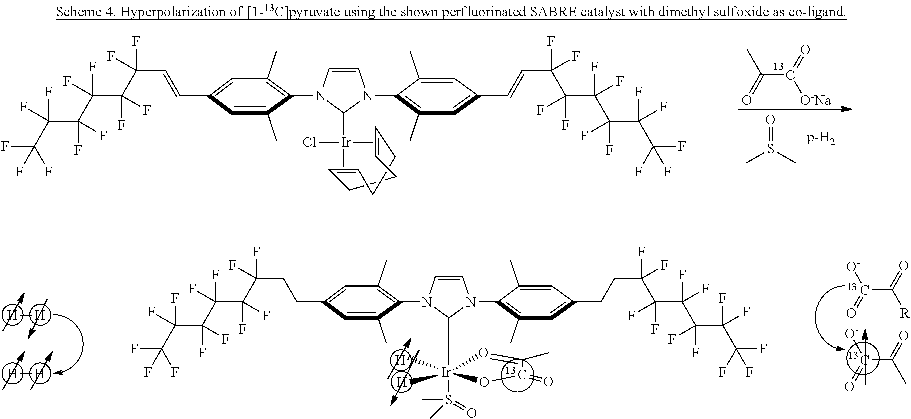

In the reaction scheme above, the efficient hyperpolarization transfer from p-H₂-derived hydrides to the ¹³C nuclear spin of [1-¹³C]pyruvate was attained by performing SABRE in sub-microtesla magnetic fields using SABRE in SHield Enables Alignment Transfer to Heteronuclei (SABRE-SHEATH) using a solution mixture of [IrCl(H)₂(DMSO)₂(F-IMes)], [1-¹³C]pyruvate) and p-H₂ in deuterated methanol.

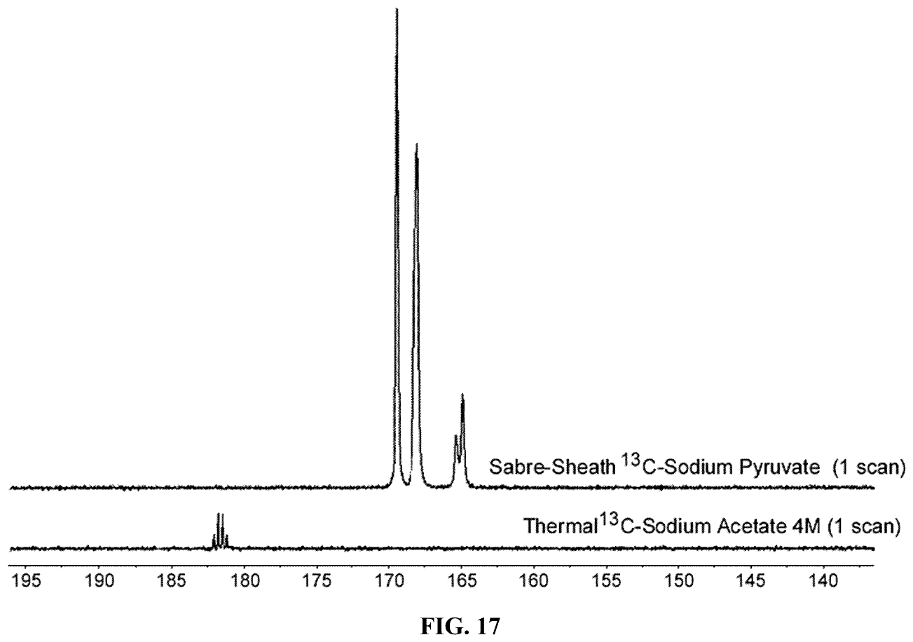

The SABRE samples were prepared in CD₃OD, using 20 mM sodium [1-¹³C]pyruvate, 7.6 mM perfluorinated SABRE catalyst shown in Scheme 4, and 50 mM dimethyl sulfoxide (DMSO), as described in Examples 6 and 7. The SABRE samples were exposed to the SABRE-SHEATH hyperpolarization conditions with the same set-up and optimum conditions described in Examples 6 and 7. The NMR tubes were pressurized (110 psi, i.e., approximately 8 bar total pressure) with p-H₂ bubbling through the solution at a flow of 90 scc/m to activate the catalyst and to ¹³C-hyperpolarize the sodium [1-¹³C]pyruvate solution. Activation of the catalyst took place for about 15 minutes at ambient temperature and magnetic field. For polarization build-up, the sample was placed in the magnetic field (typically about 0.4 μT) and a water bath at 5° C. to regulate the reaction temperature. The spectrum was acquired immediately following manual sample transfer to a 1.8 T benchtop NMR after 5 seconds, and the results are set forth in the top spectrum of FIG. 17. In addition, the bottom spectrum of FIG. 17 shows a single-scan thermally polarized ¹³C signal from 4 M sodium [1-¹³C] acetate using similar acquisition parameters. As is apparent from the results set forth in FIG. 17, the signal enhancement is a-19000 and polarization is about P(¹³C) 4.91%.

### Example 24

This example illustrates a method of hyperpolarizing a [1-¹³C]pyruvate in accordance with an aspect of the invention, using a fluorous mixture instead of only deuterated methanol.

The hyperpolarization procedure of Example 6 was repeated using a mixture of nonafluorobutyl methyl ether (NFBME) and deuterated methanol instead of only deuterated methanol.

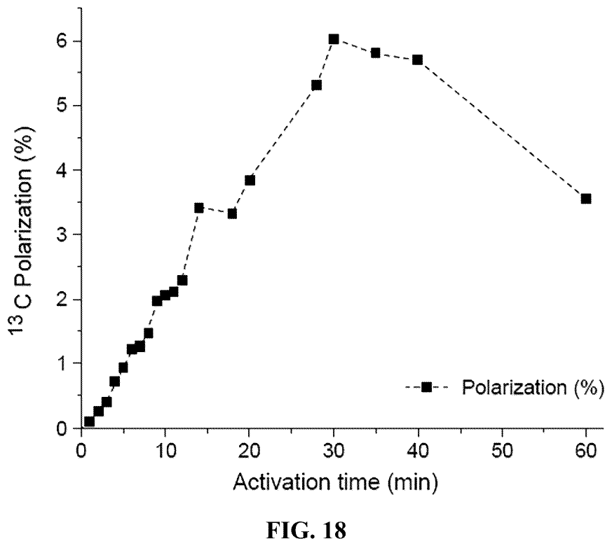

The fluorinated SABRE catalyst activation took less than 25 minutes, with the ¹³C polarization percentage shown in FIG. 18, and is performed by bubbling ˜95% p-H₂ at a flow rate of 90 standard cubic centimeters per minute (scc/m) at 8 atm p-H₂ partial pressure. The NMR samples contained about 25 mM sodium [1-¹³C]pyruvate, 7.4 mM perfluorinated SABRE catalyst of Example 4, and 40 mM dimethyl sulfoxide (DMSO) in 0.3 mL NFBME and 0.2 mL MeOD with the mixing field at 0.4 μT and a temperature of 0° C.

### Example 25

This example demonstrates the effects on hyperpolarization of [1-¹³C]pyruvate in a fluorous mixture, exhibited by changes in parahydrogen pressure and flow rate, as well as the effect of magnetic transfer field. In addition, the relaxation dynamics of the [1-¹³C]pyruvate were also studied.

Hyperpolarization of [1-¹³C]pyruvate was repeated using SABRE in SHield Enables Alignment Transfer to Heteronuclei (SABRE-SHEATH), as described in Example 24 above, and the effects of parahydrogen flow rate, as well as the effect of magnetic transfer field, were studied.

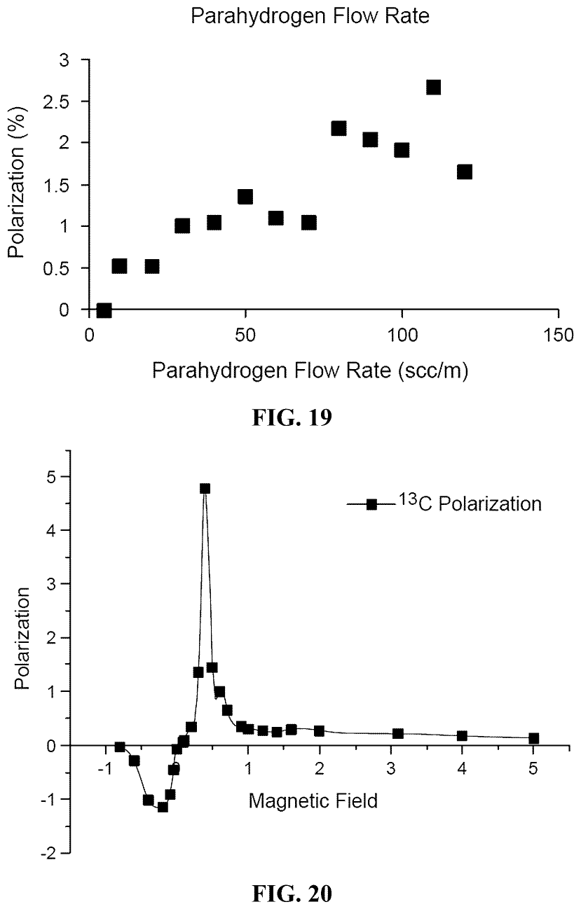

p-H₂ flow rate was evaluated and the polarization percentage results are set forth in FIG. 19. The NMR samples contained about 22 mM sodium [1-¹³C]pyruvate, 7.4 mM perfluorinated SABRE catalyst of Example 4, and 45 mM dimethyl sulfoxide (DMSO) in 0.3 mL NFBME and 0.2 mL MeOD with the mixing field at 0.4 μT and a temperature of 0° C. As is apparent from the results set forth in FIG. 19, as the parahydrogen flow rate increased, the polarization percentage also increased.

The magnetic field in the micro Tesla regime was evaluated and the ¹³C polarization transfer magnetic field at 0° C. is set forth in FIG. 20. The NMR samples contained 22 mM sodium [1-¹³C]pyruvate, 7.4 mM perfluorinated SABRE catalyst of Example 4, and 46 mM dimethyl sulfoxide (DMSO) in 0.2 mL NFBME and 0.2 mL MeOD. The p-H₂ flow rate and pressure were 50 ssc/m and 110 PSI, respectively. As is apparent from the results set forth in FIG. 20, the best polarization transfer occurs at a mixing field between 0.3 μT and 0.5 μT. The optimum mixing field is 0.4 μT.

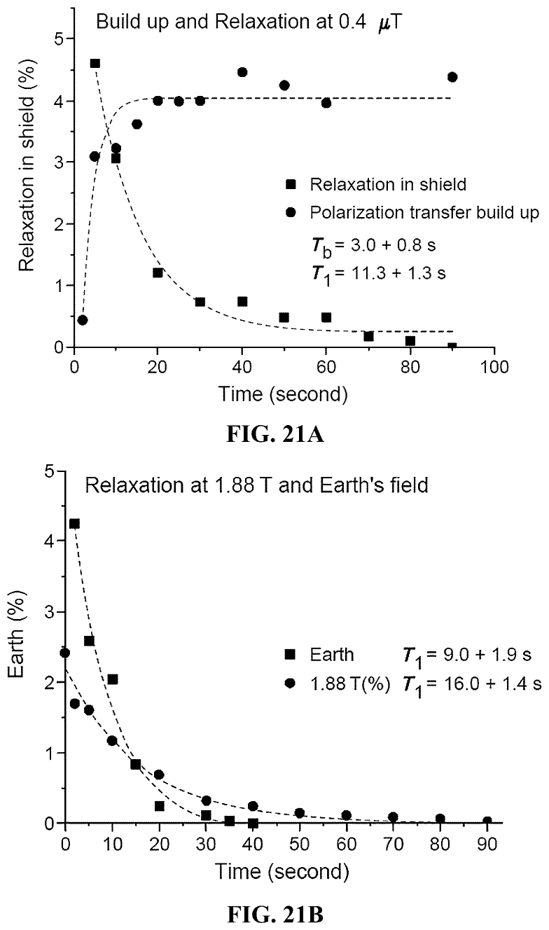

As is apparent from the results set forth in FIGS. 21A and 21B, the relaxation dynamics of [1-¹³C]-pyruvate show that the total P_{13C} (bound+free) build-up time (T_{b}=3.0±0.8 s) is substantially shorter than the corresponding T₁ value of 11.3±1.3 s, which allows to reach P_{13C} levels up to 6.02%. In addition, relaxation dynamics at earth field and 1.8 T are about the same as a non-fluorinated SABRE catalyst T₁=9.0±1.9 s and 16.0±1.4 s, respectively.

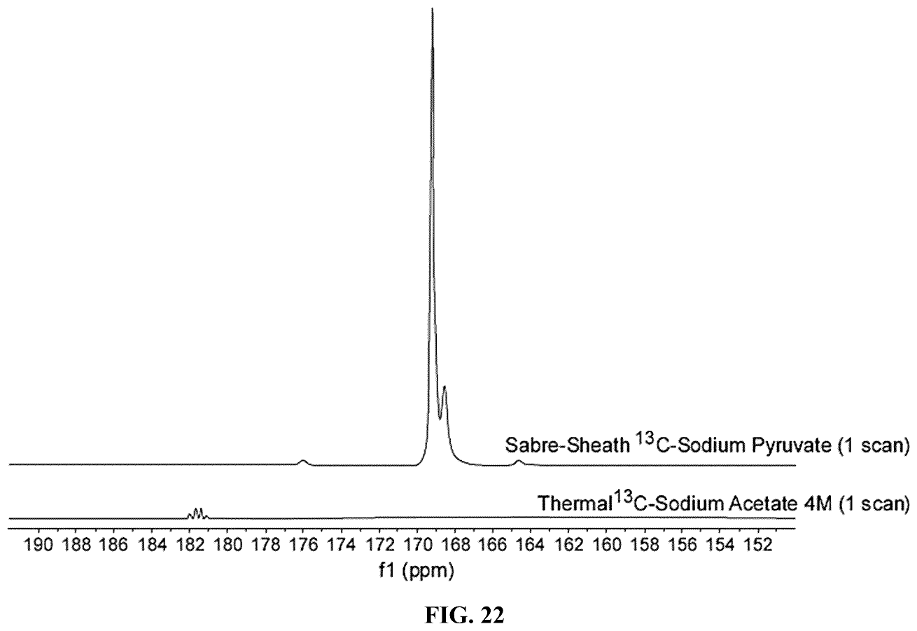

The simultaneous exchange of p-H₂ and [1-¹³C]pyruvate on activated Ir(F-IMes) catalyst leads to buildup of ¹³C hyperpolarization. In that respect, the top spectrum of FIG. 22 shows a representative spectrum of ¹³C-hyperpolarized [1-¹³C]-pyruvate with signal enhancement ε of ˜38600 fold, corresponding to P_{13C} of ˜6.02% obtained via comparison of the NMR signal intensity to a reference sample (i.e., the bottom spectrum of FIG. 22, which shows a single-scan thermally polarized ¹³C signal from 4 M sodium [1-¹³C] acetate using similar acquisition parameters). The NMR samples contained 23 mM sodium [1-¹³C]pyruvate, 7.4 mM perfluorinated SABRE catalyst of Example 4, and 46 mM dimethyl sulfoxide (DMSO) in 0.2 mL NFBME and 0.2 mL MeOD. The p-H₂ flow rate and pressure were 90 ssc/m and 110 PSI, respectively, with the mixing field at 0.4 μT and a temperature of 0° C.

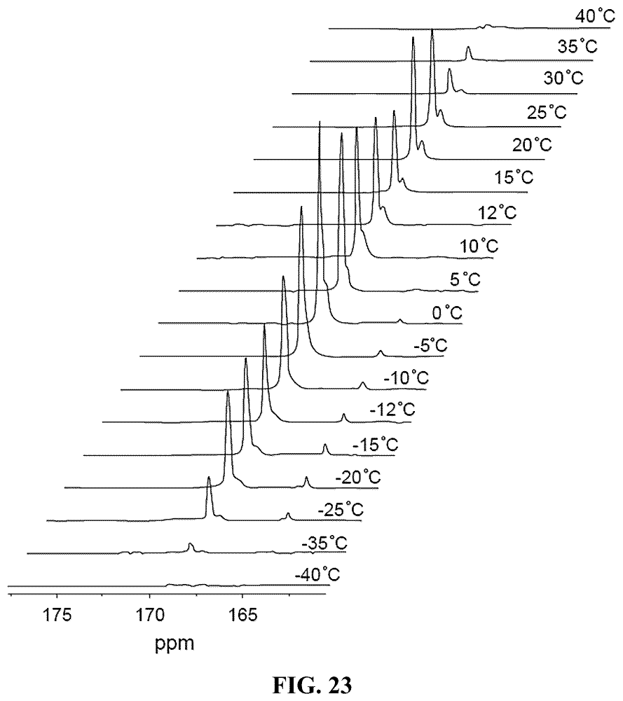

Temperature has a profound effect on the exchange rates of [1-¹³C]pyruvate on Complex 3b of FIG. 7. In recent work, Adelabu et al. (ChemPhysChem, 23, e202100839 (2022)) showed that the monotonic disappearance of free HP resonance at low temperatures happened due to the slow exchange rate of Complex 3b into the free state. At room temperature (e.g., 22° C.), the exchange of [1-¹³C]pyruvate with the polarization transfer complex was faster, leading to hyperpolarization of both free and bound 3b species in the expected pyruvate:pre-catalyst ratio. In order to rapidly release the HP pyruvate from 3b, the HP solution was rapidly warmed up then the sample was inserted in the NMR detector (TomHon et al., J. Am. Chem. Soc., 144(1) 282˜287 (2022)). FIG. 23 shows a variable temperature SABRE-SHEATH experiment using the saturated perfluorinated SABRE catalyst of Example 4. The NMR samples (in nonafluorobutyl methyl ether (NFBME) and deuterated methanol) contained in 23 mM sodium [1-¹³C]pyruvate, 7.4 mM perfluorinated SABRE catalyst of Example 4, and 46 mM dimethyl sulfoxide (DMSO), wherein the mixing field was at 0.4 μT, and the parahydrogen pressure and flow rate were set at 110 PSI and 90 scc/m, respectively.

As demonstrated by FIG. 23, the exchange rate of Complex 3b into the free state is faster even at low temperature, such as −10° C., where most of the HP [1-¹³C]pyruvate is in a free state. By decreasing the temperature of ¹³C SABRE-SHEATH to 0° C., P_{13C} maximum is achieved. The exchange remains fast enough to build-up preferentially the “free” HP [1-¹³C]pyruvate over Complex 3b.

### Example 26

This example illustrates an exemplary method for isolating hyperpolarized sodium [1-¹³C]pyruvate, which includes biphasic extraction with an aqueous phase and a fluorinated phase.

The hyperpolarization procedure of Example 6 was repeated using a mixture of nonafluorobutyl methyl ether (NFBME) and deuterated methanol instead of only deuterated methanol. The SABRE sample was prepared with 23 mM sodium [1-¹³C]pyruvate, 7.4 mM perfluorinated SABRE catalyst of Example 4, and 46 mM dimethyl sulfoxide (DMSO) in 0.2 mL NFBME and 0.2 mL MeOD, wherein the mixing field was at 0.4 μT, and the parahydrogen pressure and flow rate were set at 110 PSI and 90 scc/m, respectively.

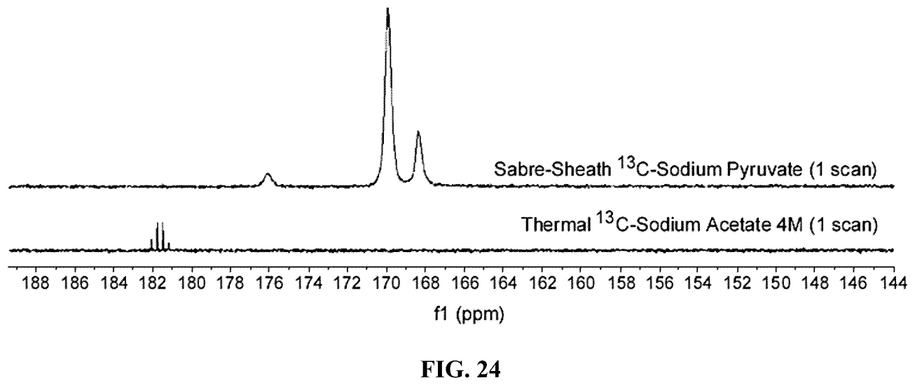

After the hyperpolarization procedure was completed, the sample was rapidly removed from the 0.40 μT field and transferred inside the NMR spectrometer at 1.8 T at room temperature. The NMR sample was depressurized and 400 μL of heavy water (D₂O) was added to the solution in order to drive the hyperpolarized sodium [1-¹³C]pyruvate into the aqueous phase. The pyruvate signal in the aqueous phase was collected, but the separation led to the formation of an emulsion, indicating that both bound and free pyruvate are present. See FIG. 24. In that respect, the top spectrum of FIG. 24 shows a representative spectrum of ¹³C-hyperpolarized [1-¹³C]-pyruvate with signal enhancement ε of ˜10800 fold, corresponding to P_{13C} of ˜1.68% obtained via comparison of the NMR signal intensity to a reference sample (i.e., the bottom spectrum of FIG. 24, which shows a single-scan thermally polarized ¹³C signal from 4 M sodium [1-¹³C] acetate using similar acquisition parameters).

The aqueous phase containing the sodium [1-¹³C]pyruvate was evacuated and tested for iridium content and pyruvate concentration. The ICP-MS study showed a content of 637 ppb of iridium and the LC-MS showed about 50-75% of the sodium [1-¹³C]pyruvate concentration was collected after filtration.

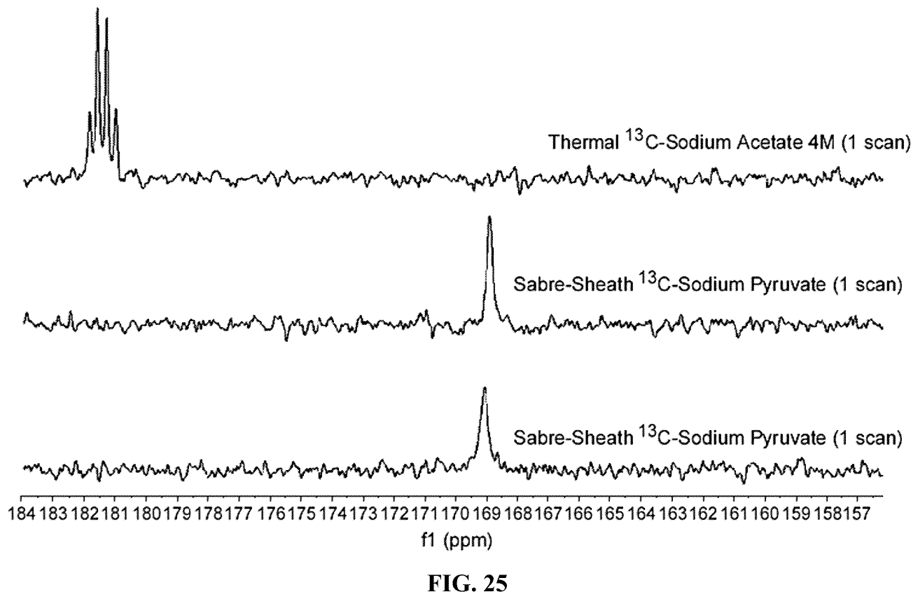

The fluorous mixture containing the perfluorinated SABRE catalyst in NFBME and MeOD was re-used for further sodium [1-¹³C]pyruvate hyperpolarization. After evacuation of the water phase, a solution containing 23 mM sodium [1-¹³C]pyruvate and 47 mM dimethyl sulfoxide in 0.2 mL CD₃OD was added to the fluorous mixture containing the perfluorinated SABRE catalyst from 4 days earlier. The hyperpolarization of sodium [1-¹³C]pyruvate was repeated, and showed a polarization of the [1-¹³C] pyruvate with a signal enhancement ε of ˜3090 fold, corresponding to P_{13C} of ˜0.48%, which was obtained via comparison of the NMR signal intensity to a reference sample, as shown in FIG. 25. The polarization of [1-¹³C]pyruvate is repeatable and showed about the same polarization level, demonstrating that the perfluorinated SABRE catalyst remains active for at least 4 days after initial use.

### Example 27

This example illustrates a synthesis of 1,3-dimesityl-4,5-bis(2-(perfluorophenyl)propyl)-4,5-dihydro-1H-imidazol-3-ium, triflate salt:

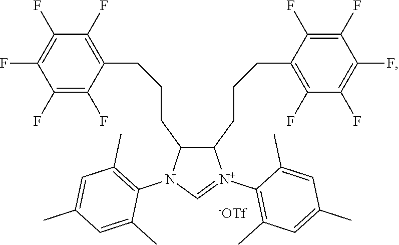 [SMILES: Cc1cc(C)c(N2C=[N+](c3c(C)cc(C)cc3C)C(CCCc3c(F)c(F)c(F)c(F)c3F)C2CCCc2c(F)c(F)c(F)c(F)c2F)c(C)c1 ; "-OTf"]

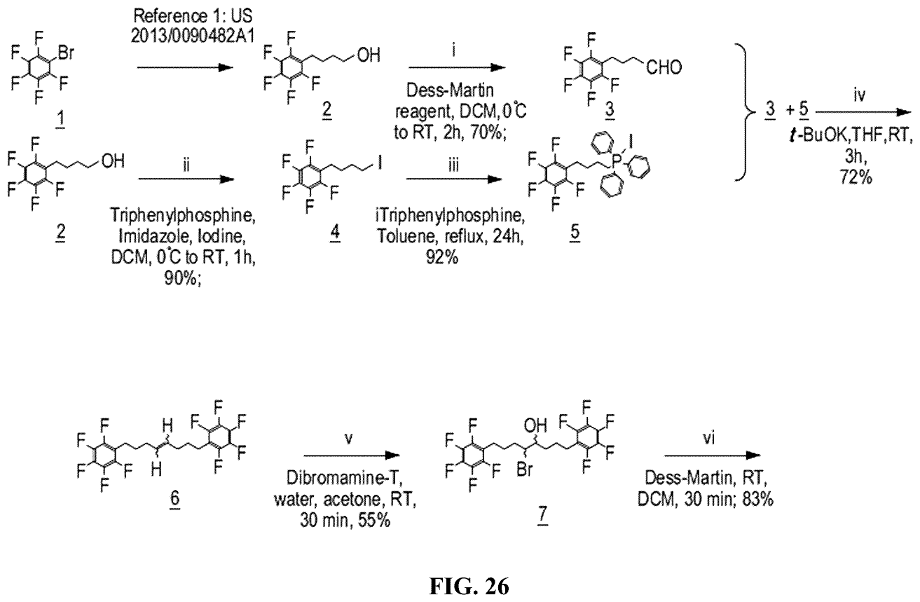

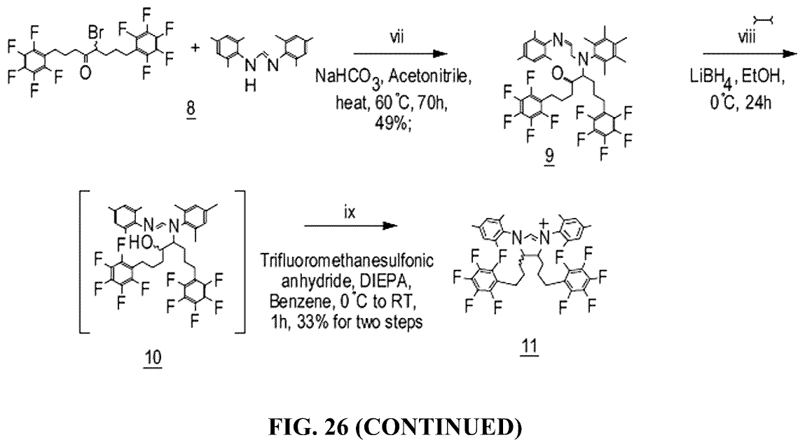

which was prepared in accordance with the synthesis sequence set forth in FIG. 26.

4-Pentafluorophenylbutanal (3). To a solution of the alcohol (2) (4.8 g, 20.0 mmol) in DCM (40 mL), was added Dess-Martin periodinane (16.96 g, 40.0 mmol) portionwise at 0° C. while being stirred under argon (5 minutes). The reaction was continued until the starting alcohol was consumed (2 hours). The resulting solution was concentrated to about 10.0 mL and adsorbed onto 10.0 g of silica and dried to a free flowing powder. The silica with the crude product was applied to a silica column (120.0 g), and elution with 5% ethyl acetate in hexanes yielded the product as colorless oil. Yield: 3.33 g (70%). ¹H NMR (CDCl₃): ¹H NMR (400 MHz, CDCl₃) δ 9.71 (t, J=1.3 Hz, 1H), 2.69 (tt, J=7.9, 1.8 Hz, 2H), 2.44 (td, J=7.3, 1.2 Hz, 2H), 1.86 (p, J=7.3 Hz, 2H). ¹³C NMR (101 MHz, CDCl₃) δ 200.89, 42.81, 21.49.

1,2,3,4,5-Pentafluoro-6-(4-iodobutyl)benzene (4). An ice-cooled solution of the alcohol (2) (16.0 g, 66.67 mmol) was stirred with imidazole (5.89 g, 86.67 mmol) and triphenylphosphine (20.96 g, 80.0 mmol) in DCM (100 mL) under argon. Iodine (20.32 g, 80.0 mmol) was added portionwise over a period of 15 minutes at 0° C. while being vigorously stirred until a slight yellow color persisted. The reaction mixture was diluted with hexanes (300 mL) and filtered. The filtrate was washed with saturated sodium thiosulfate (3×50 mL), water (3×100 mL), and dried with sodium sulfate. The clear solution was filtered and concentrated to an oil that was chromatographed over silica gel (220 g). Elution with hexanes yielded compound (4) as colorless oil. Yield: 21.0 g (90%). ¹H NMR (400 MHz, cdcl₃) δ 3.20 (t, J=6.8 Hz, 1H), 2.73 (tt, J=7.5, 1.8 Hz, 1H), 1.86 (dq, J=8.6, 6.8 Hz, 1H), 1.76-1.66 (m, 1H). ¹³C NMR (101 MHz, cdcl₃) δ 37.84, 30.01, 21.27, 5.39.

Iodo-(4-(pentafluorophenyl)butyl)triphenyl-λ⁵-phosphane (5). A solution of the iodobutane (4) (21.0 g, 60.0 mmol) and triphenylphosphine (17.29 g, 66.0 mmol) was refluxed under argon for 24 hours. The precipitated solid was filtered and washed with anhydrous ether and dried under high vacuum for 20 hours. Yield: 33.78 g (92%). ¹H NMR (400 MHz, CDCl₃) δ 7.73 (m, 15H), 3.92, 3.86 (m, 2H), 2.76, 2.74 (m, 2H), 2.07, 2.025 (m, 2H), 1.6 (m, 2H). ¹³C NMR (101 MHz, CDCl₃) δ 135.23, 135.20, 133.75, 133.65, 130.65, 130.53, 118.32, 117.46, 29.43, 29.27, 23.05, 22.55, 21.78, 21.74, 21.66.

Compound 6. To a solution of the phosphonium salt (5) (6.79 g, 11 mmol) in anhydrous THF (40 mL) was added potassium tert-butoxide in THF (2M, 12.5 mL, 25.0 mmol) dropwise and stirred under argon for 30 minutes. Aldehyde (3) (2.2 g, 9.24 mmol) in THF (5 mL) was added dropwise at room temperature and stirred for 3 hours. The solution was concentrated, and purified on flash silica (120 g). Elution with hexanes yielded the product as a colorless oil as mixture of cis/trans isomers (90:10). Yield: 3.2 g (72%). ¹H NMR (400 MHz, CDCl₃) δ 5.41 (m, 2H), 2.72, 2.68 (m, 4H), 2.12, 2.10, 2.06 (m, 4H), 1.64 (m, 4H).¹³C NMR (101 MHz, CDCl₃) δ 129.46, 29.12, 26.76, 21.94

Compound 7. Olefin (6) (3.6 g, 8.1 mmol) in acetone (20 mL) and water (0.5 mL) was cooled to 0° C., and dibromamine-T (2.93 g, 8.91 mmol, Org. Biomol. Chem., 8, 1424-1430 (2010)) was added with vigorous stirring. After the addition, the solution was brought to room temperature and stirred until the starting material disappeared (30 minutes). The solution was quenched with solid sodium thiosulfate (2.0 g) and stirred until the color of bromine was completely discharged. The mixture was concentrated under reduced pressure, and the residue was diluted with water (50 mL) and extracted with EtOAc (3×30 mL). The combined organic layers were dried (MgSO₄), filtered, and concentrated under reduced pressure. The residue was chromatographed over flash silica (80 g) and eluted with 10% EtOAc in hexanes to yield the bromohydrin (7) as mixture of diastereomers and as colorless oil. Yield: 2.41 g (55%). ¹H NMR (400 MHz), CDCl₃) δ 4.02 (m, 1H), 3.48 (m, 1H), 2.73 (4H, CH₂—C₆F₅), 1.6˜2.0 (m, 9H, —CH₂— and OH)). ¹³C NMR (101 MHz, CDCl₃) δ 143.76, 138.35, 129.62, 126.82, 59.74, 57.12, 35.29, 34.50, 27.39, 25.48, 21.74, 21.34.

Compound 8. The bromohydrin (7) (2.2 g, 4.06 mmol) was dissolved in DCM (10.0 mL) and stirred with 5.0 g of molecular sieves. Dess-Martin reagent (3.44 g, 8.12 mmol) was added and stirred until the starting material was completely consumed (30 minutes). The whole mixture was adsorbed onto flash silica (20.0 g) and dried to a free flowing powder. The silica with the crude product was loaded onto a flash silica (80.0 g) column and eluted with 0 to 20% EtOAc in hexanes over 20 minutes (80.0 mE/min; elution rate). Bromoketone (8) was eluted at 5% EtOAc in hexanes as a colorless syrup. Yield: 1.82 g (83%). ¹H NMR (400 MHz, CDCl₃) δ 4.25 (dd, J=8.3, 6.0 Hz, 1H), 2.94˜2.81 (m, 1H), 2.80-2.68 (m, 4H), 2.67˜2.52 (m, 1H), 2.09-1.86 (m, 4H), 1.86-1.59 (m, 2H). ¹³C NMR (101 MHz, CDCl₃) δ 202.53, 52.19, 38.18, 32.31, 26.87, 23.21, 21.62, 21.45.

Compound 9. Sodium bicarbonate (0.56 g, 6.68 mmol) was added to a stirred solution of bromoketone (8) (1.8 g, 3.34 mmol) in anhydrous acetonitrile (10.0 mL). Trimethylaniline formamidine (0.94 g, 3.34 mmol) was then added to the stirred solution as a solid, and the reaction mixture was heated to 60° C. with the exclusion of moisture. After the consumption of the bromoketone (70 hours), the reaction mixture was filtered, concentrated under reduced pressure, and chromatographed over flash silica (80 g). Elution with 20% DCM in hexanes yielded the ketoamidine (10) as a colorless syrup. Yield: 1.2 g (49%). ¹H NMR 6 (CDCl₃) δ.91 9s, 1H), 6.81 (s, 1H), 6.71 (s, 1H), 6.68 (s, 2H), 4.53 (m, 1H), 3.08 (m, 1H), 2.57 (m, 2 h), 2.43 (m, 2H), 2.35 (m, 2H), 2.36 (s, 3 h), 2.2 (s, 3H), 2.13 (s, 3H), 2.01 (s, 3H), 1.92 (s, 6H), 1.8 (m, 2H). ¹³C NMR (101 MHz, CDCl₃) δ 208.19, 151.90, 138.19, 131.45, 129.47, 128.74, 64.33, 60.38, 41.49, 28.39, 25.50, 22.96, 20.56, 18.44, 18.41, 18.23, 14.18. MS: 739.1 [M+H].

Compound 11 (triflate salt). To an ice-cooled solution of the keto amidine (0.994 g, 1.35 mmol) in ethanol, lithium borohydride (2M in THF, 0.675 ml, 1.35 mmol) was added and stirred under argon for 24 h at 0-5° C. The reaction mixture was poured into 50 ml of water and extracted with ethyl acetate (3×50 ml). The combined organic layers were dried (sodium sulfate), and filtered, and concentrated to a paste at room temperature. The above paste was dissolved in 5.0 ml of anhydrous benzene and cooled to 0° C. DIPEA (0.523 g, 4.05 mmol) was added and stirred under argon. Trifluoromethanesulfonic anhydride (1.104 g, 1.34 mmol) was then added dropwise and stirring was continued for 1 h. LC/MS indicated the complete consumption of peak corresponding to 741. The mixture was adsorbed onto 10.0 g of silica gel and dried to a free flowing powder. The silica with the crude product was loaded onto an empty loading column placed on the top of a 80.0 g silica column and chromatographed. Elution with 4% MeOH in DCM yielded the product as a mixture of cis/trans isomers and as brown paste. Yield: 0.338 g (33%). ¹H NMR (CDCl₃) δ 8.82 (s, 1H), 6.93 (s, 4H), 4.15 (m, 2H), 2.61 (m, 4H), 2.25, 2.23, 2.21 (3S, 18H), 1.77 (m, 4H), 1.41 (m, 4H). ¹³C NMR (101 MHz, CDCl₃) δ 159.41, 141.00, 135.66, 134.43, 130.60, 129.96, 128.58, 68.76, 66.17, 32.48, 26.91, 24.65, 21.71, 20.97, 18.38, 18.11. MS: 723.0 [M+H].

### Example 28

This example illustrates the synthesis of 1,3-dimesityl-4,5-bis(4,4,5,5,6,6,7,7,8,8,9,9,9-tridecafluorononyl)-1H-imidazol-3-ium triflate:

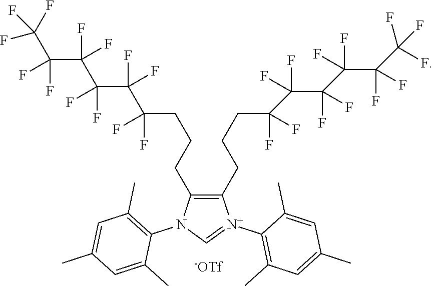 [SMILES: Cc1cc(C)c(-n2c[n+](-c3c(C)cc(C)cc3C)c(CCCC(F)(F)C(F)(F)C(F)(F)C(F)(F)C(F)(F)C(F)(F)F)c2CCCC(F)(F)C(F)(F)C(F)(F)C(F)(F)C(F)(F)C(F)(F)F)c(C)c1 ; "-OTf"]

### 1,3-dimesityl-4,5-bis(4,4,5,5,6,6,7,7,8,8,9,9,9-tridecafluorononyl)-1H-imidazol-3-ium triflate was prepared according to the steps set forth in Scheme 5

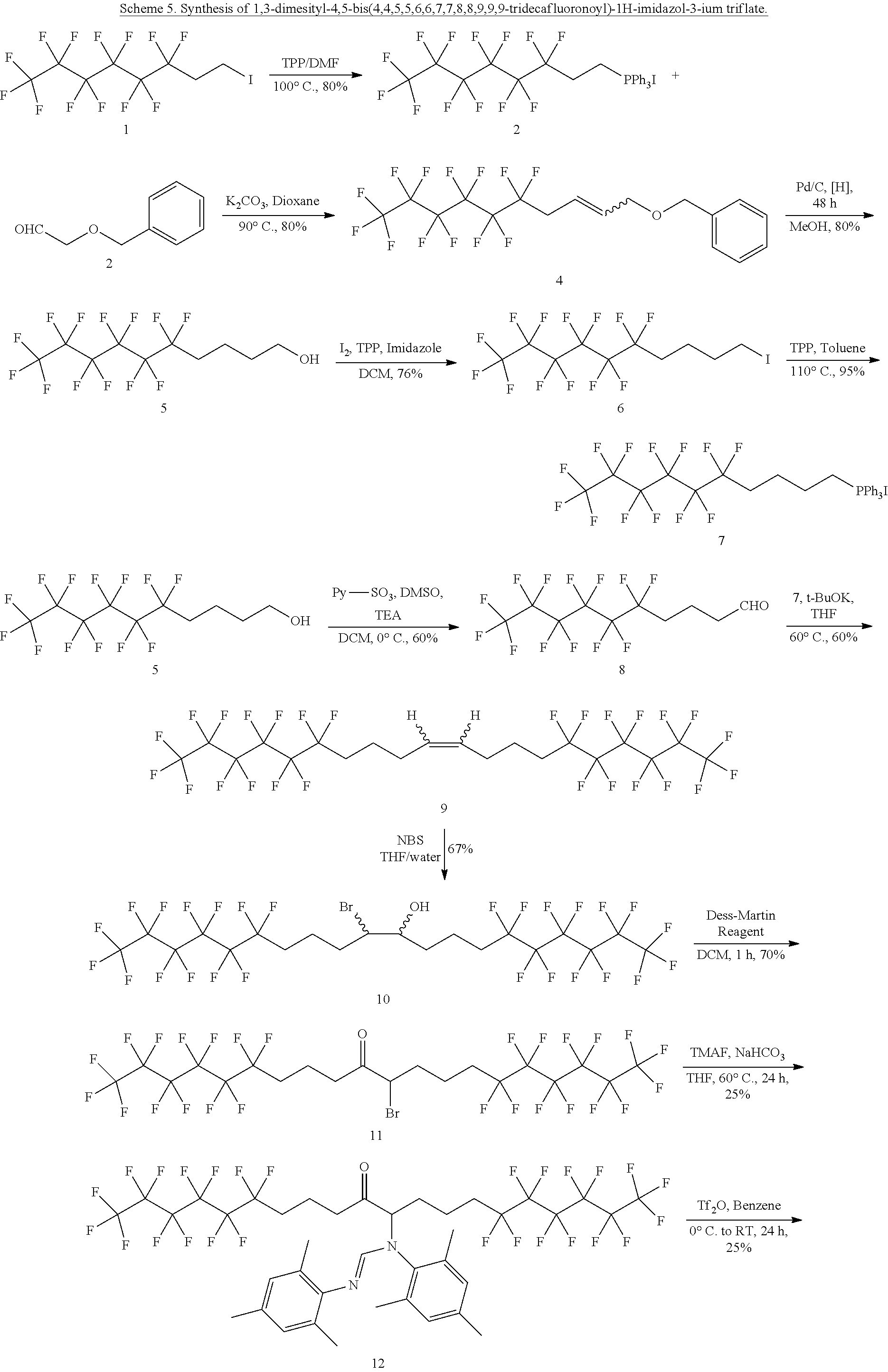

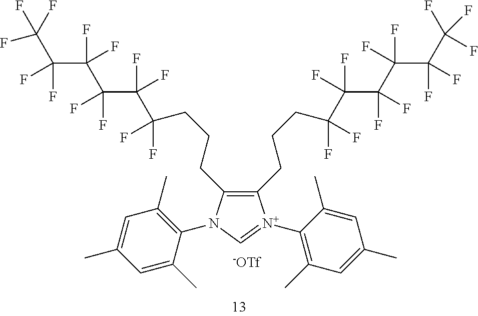 [SMILES: Cc1cc(C)c(-n2c[n+](-c3c(C)cc(C)cc3C)c(CCCC(F)(F)C(F)(F)C(F)(F)C(F)(F)C(F)(F)C(F)(F)F)c2CCCC(F)(F)C(F)(F)C(F)(F)C(F)(F)C(F)(F)C(F)(F)F)c(C)c1 ; "-OTf"]

A flask was charged with Ph₃P (7.64 g, 29.13 mmol), ICH₂CH₂C₆F₁₃ (1, 12.40 g, 26.16 mmol) and dimethylformamide (DMF) (15 ml). The mixture was stirred vigorously for 24 hours at 105° C. The DMF was removed by vacuum and the waxy solid was triturated with diethyl ether (100 ml), collected by filtration, washed with diethyl ether, and dried by vacuum to give phosphonium salt (2) as a white solid (16.95 g, 88%) [Rocaboy et al. (J. Phys. Org. Chem., 13: 596-603 (2000))].

A round-bottomed flask was charged with 2-benzyloxybenzaldehyde (3, 7.5 g, 50.0 mmol), phosphonium salt 2 (44.0 g, 60.0 mmol), K₂CO₃ (10.0 g, 72.4 mmol), reagent-grade 1,4-dioxane (100 ml), and water (2.0 ml) and fitted with a condenser (no inert atmosphere). The mixture was stirred at 95° C. for 20 hours. The volatiles were removed by reduced pressure, and CH₂Cl₂ (200 ml) and water (50 ml) were added to the orange residue. The layers were separated ad the aqueous layer was extracted with CH₂Cl₂ (3×50 ml) and the CH₂Cl₂ layers were combined and dried (MgSO₄). The solvent was removed by reduced pressure and the oily solid was placed over 100.0 g of silica gel plug and eluted with hexanes. The solvent was removed by reduced pressure to give the olefin (4) as colorless oil (17.0 g, 88%, 90:10 Z/E). ¹H NMR (400 MHz, CDCl₃) δ 7.41-7.28 (m, 5H), 6.02-5.93 (m, 1H), 5.64 (dt, J=9.5, 7.3 Hz, 1H), 4.52 (s, 2H), 4.09 (dd, J=6.3, 1.6 Hz, 2H), 2.99˜2.82 (m, 2H).

Olefin 4 (17.0 g) was dissolved in methanol (100.0 ml), and palladium on carbon (10%, 50% by weight water, 3.4 g) was added and hydrogenated at 60 psi for 24 hours. The reaction mixture was filtered through a pad of celite and concentrated to yield the alcohol (5) as a colorless oil (14.0 g, 82%) [Tetrahedron Letters, 43, 6141-6143 (2002)].

The alcohol 5 (13.5 g, 34.4 mmol), triphenylphosphine (10.8 g, 41.3 mmol), and imidazole (3.52 g, 51.6 mmol) were dissolved in anhydrous dichloromethane (100.0 ml) and cooled in an ice bath. Iodine (10.5 g, 41.3 mmol) was added as a solid in portions with stirring over 30 minutes. Additional iodine (0.1 equiv.) was added until a pink color persisted. The solution was quenched with saturated sodium dithionite (100.0 ml) and the organic layer was separated. The aqueous layer was extracted with dichloromethane (3×50.0 ml), and the combined organic layer was washed with water, and dried (Na₂SO₄). The solution was filtered, concentrated under reduced pressure, and the residue was purified by silica gel column. Elution with hexanes yielded the iodide (6) as colorless oil (13.2 g, 76.4%). Loss of iodine was noticed while on standing at RT, and the iodide was taken to next step immediately. ¹H NMR (400 MHz, CDCl₃) δ 3.20 (t, J=6.8 Hz, 2H), 2.19-1.85 (m, 2H), 1.75 (ddd, J=15.6, 9.7, 6.0 Hz, 2H).

The iodide 6 (2.9, 5.78 mmol) and triphenylphosphine (1.57 g, 6.0 mmol) were dissolved in anhydrous toluene (25.0 ml) and refluxed for 48 hours under argon. The solution was cooled and concentration under reduced pressure yielded a gum that was triturated with 250 mL of anhydrous ether. The precipitated white solid was filtered and washed with ether and dried under vacuum to yield the phosphonium salt (7) as colorless solid (4.4 g, 99%). ¹H NMR (400 MHz, CDCl₃) δ 3.20 (t, J=6.8 Hz, 2H), 2.1 (m, 2H), 1.92 (dt, J=14.5, 7.0 Hz, 2H), 1.75 (tt, J=10.8, 6.2 Hz, 2H). ¹³C NMR (101 MHz, CDCl₃) δ 135.23, 133.71, 130.65, 130.53, 118.32, 117.46, 114.10, 29.43, 29.26, 23.05, 22.55, 21.78, 21.74, 21.66.

A solution of the alcohol 5 (1.176 g, 3.0 mmol) and triethylamine (1.52 g, 15.0 mmol) was cooled to 0° C. DMSO (2.34 g, 30.0 mmol) was added and stirred, and pyridine-sulfur trioxide complex (1.52 g, 15.0 mmol) was then added in portions over 5 minutes. The resulting mixture was stirred at 0° C. until the alcohol was completely consumed (1 hour). The mixture was diluted with 30 ml of DCM and washed with 2×20 ml of water followed by saturated sodium chloride, dried (potassium carbonate), filtered, concentrated under reduced pressure at room temperature, and the residue was chromatographed over 40 g of flash silica. Elution with 10% ethyl acetate in hexanes yielded the aldehyde (8) as a colorless oil (0.72 g, 61.54%). ¹H NMR (400 MHz, CDCl₃) δ 9.80 (t, J=1.0 Hz, 1H), 2.61 (td, J=7.0, 1.0 Hz, 2H), 2.22˜2.03 (m, 4H), 2.03-1.90 (m, 2H).

A suspension of the phosphonium salt 7 (1.693 g, 2.215 mmol) in THF (10.0 ml) was cooled to −40° C., and potassium-t-butoxide in THF (1 M solution, 2.4 mL) was added dropwise and stirred until the solids went into solution to give an orange color. The solution was warmed to 0° C. and stirred for 30 minutes. The aldehyde 8 (0.72 g, 1.845 mmol) was then added through a cannula under a positive pressure of argon, and after the addition, the temperature was raised to 50° C., and the reaction was continued under argon for 24 hours. The reaction was quenched with 5.0 ml of saturated ammonium chloride and extracted with 3×30 ml of EtOAc. The combined organic layers were washed with water, saturated sodium chloride, and dried (MgSO₄). The resulting residue was filtered, and concentrated under reduced pressure, and purified on a 40.0 g of flash column. Elution with hexanes yielded the olefin (9) as mixture of cis/trans isomers as colorless oil (0.828 g, 60%). ¹H NMR (400 MHz, CDCl₃) δ 5.4 (m, 2H), 2.61 (m, 2.09, 8H), 1.70 (m, 4H).

To a solution of the olefin 9 (804 mg, 1.074 mmol) in water and THF (6 ml, 1:3), N-Bromosuccinimide (crystallized, 385.2 mg, 2.15 mmol) was added in portions over 10 minutes and stirred at room temperature for 2 hours. TLC indicated the complete disappearance of olefin. The mixture was quenched with 5.0 ml of sodium bisulfite and extracted with 2×20 mL of EtOAc, washed with water, and dried (sodium sulfate). The solution was filtered, concentrated, and the residue was chromatographed over 24 g of silica. Elution with 1:1 hexanes/DCM yielded the bromohydrin (10) as a mixture of diastereomers (612.0 mg, 67.4%). ¹H NMR (400 MHz, CDCl₃) δ 4.03 (s, OH), 3.95 (dd, J=9.2, 4.0 Hz, 1H), 3.69 (s, OH), 3.47 (d, J=7.6 Hz, 1H), 2.13-1.53 (m, 11H). ¹⁹F NMR (376 MHz, CDCl₃) δ-80.85 (t, J=10.0 Hz), −113.91-−114.73 (m), −121.95 (p, J=13.5 Hz), −122.74-−123.08 (m), −123.56 (dq, J=23.6, 11.3 Hz), −126.18 (td, J=14.7, 6.2 Hz). ¹³C NMR (101 MHz, CDCl₃) δ 115.74, 77.18, 73.49, 63.16, 34.90, 34.86, 30.86, 30.64, 30.42, 30.21, 29.99, 18.92, 17.77.

To an ice-cooled solution of bromohydrin 10 (612.0 mg, 0.724 mmol) in DCM (10.0 ml), Dess-Martin reagent (460.7 mg, 1.086 mmol) was added in portions with stirring over 5 minutes. The solution was stirred at room temperature until all the alcohol was consumed (TLC, 1 hour). The reaction mixture was diluted with 25.0 mL of EtOAc and washed with 3×25 ml of saturated sodium bicarbonate. The organic layer was dried (sodium sulfate), filtered, and concentrated to a paste. The crude mixture was purified by flash column chromatography (40.0 g). Elution with 30% DCM in hexanes yielded bromoketone (11) as a colorless gum (440.0 mg, 70%). ¹H NMR (400 MHz, CDCl₃) δ 4.19 (dd, J=8.3, 5.9 Hz, 1H), 3.00-2.84 (m, 1H), 2.71˜2.58 (m, 1H), 2.15-1.86 (m, 8H), 1.78 (tdd, J=15.7, 8.4, 4.9 Hz, 1H), 1.68-1.52 (m, 1H). ¹⁹F NMR (376 MHz, cdcl₃) δ-80.93, −114.31, −121.95, −122.88, −123.59, −126.26. ¹³C NMR (101 MHz, cdcl₃) δ 202.15, 51.89, 37.92, 32.27, 30.48, 30.26, 30.03, 29.94, 29.72, 29.50, 18.41, 18.37, 14.86, 14.82, 14.77.

To a solution of the bromoketone 11 (440.0 mg, 0.522 mmol) in acetonitrile (5.0 mL), N,N′-dimesitylformamidine was added as a solid (176.0 mg, 0.63 mmol) followed by NaHCO₃(132.0 mg, 1.57 mmol). The solution was heated with stirring at 60° C. under argon for 24 hours. The mixture was filtered, washed with 3×10 mL of EtOAc, and the filtrates were concentrated and purified by column chromatography (40.0 g of flash silica). Elution with 1:1 hexanes/DCM yielded amidino ketone (12) as a colorless gum. Yield: 138.2 mg (25.4%). ¹H NMR (400 MHz, cdcl₃) δ 7.06 (s, 1H), 6.94 (s, 1H), 4.65 (s, 1H), 3.30 (s, 1H), 2.66˜2.49 (m, 1H), 2.28 (s, 3H), 2.20 (s, 3H), 2.15 (s, 3H), 2.07 (s, 6H), 1.93 (d, J=8.1 Hz, 3H), 1.46 (d, J=17.0 Hz, 3H). ¹⁹F NMR (376 MHz, cdcl₃) δ-80.64-−81.09 (m), −114.35, −114.72, −122.06, −122.98, −123.66 (d, J=107.9 Hz), −126.22. ¹³C NMR (101 MHz, cdcl₃) δ 207.98, 152.08, 138.26, 131.57, 129.71, 128.86, 128.47, 77.31, 77.19, 76.99, 76.67, 64.30, 41.52, 29.68, 28.74, 20.52, 18.52, 18.50, 18.28, 16.95, 14.29. M>S.: [M+H] 1043.0.

To a solution of the amidino ketone 12 (100.0 mg, 0.1 mmol) in anhydrous benzene (0.5 ml) and diisopropylethylamine (65.0 mg, 0.5 mmol) was added and cooled in ice. Trifluoromethanesulfonic anhydride (142.0 mg, 0.5 mmol) was added and stirred under argon. The reaction mixture was allowed to warm to room temperature and stirred overnight (20 hours). The solution was adsorbed on to 5.0 g of flash silica and dried to a free-flowing powder under vacuum. The silica with the crude product was loaded on to a flash silica column (40.0 g) and eluted with 2% methanol in dichloromethane to yield the product as a brownish gum. The gum was triturated with isopropyl ether (5.0 mL) to yield an off-white solid that was filtered and washed with the same solvent (3×5 0.0 mL). The collected solid was dried under vacuum to yield the product (13) as an off-white powder (triflate salt) (26.4 mg, 25%). ¹H NMR (400 MHz, CDCl₃) δ 9.25 (s, 1H), 7.11 (s, 4H), 2.70-2.62 (m, 4H), 2.39 (s, 6H), 2.37˜2.21 (m, 2H), 2.11 (s, 12H), 2.08-1.97 (m, 2H), 1.74-1.63 (m, 4H). ¹⁹F NMR (376 MHz, CDCl₃) δ-78.85, −80.91, −114.11, −122.05, −122.99, −123.49, −126.33. ¹³C NMR (101 MHz, CDCl₃) δ 142.03, 137.59, 134.28, 131.81, 130.28, 128.13, 30.26, 30.04, 29.82, 22.55, 21.13, 19.71, 17.58, 17.36. M. S: [M+H] 1025.0.

### Example 29

This example illustrates the synthesis of 1,3-dimesityl-4,5-bis(4,4,5,5,6,6,7,7,8,8,9,9,9-tridecafluorononyl)-1H-imidazol-3-ium chloride:

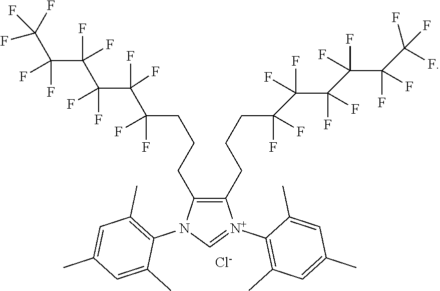 [SMILES: Cc1cc(C)c(-n2c[n+](-c3c(C)cc(C)cc3C)c(CCCC(F)(F)C(F)(F)C(F)(F)C(F)(F)C(F)(F)C(F)(F)F)c2CCCC(F)(F)C(F)(F)C(F)(F)C(F)(F)C(F)(F)C(F)(F)F)c(C)c1 ; [Cl-]]

1,3-dimesityl-4,5-bis(4,4,5,5,6,6,7,7,8,8,9,9,9-tridecafluorononyl)-1H-imidazol-3-ium triflate (100 mg, 94.2 μmol) from Example 28 was dissolved in MeOH (1.5 mL) and passed through a short column of ion exchange resin Amberlite 400. The column was washed with MeOH until no spot was visible via TLC under UV. The solvent was removed, and the resulting yellowish solid was dried with a vacuum pump to yield product (0.1 g, 99%). MS (ESI) m/z (%): 999 [M+H]⁺(100).

### Example 30

This example illustrates a method of synthesis of a fluorinated SABRE catalyst containing a transition metal in accordance with an aspect of the invention.

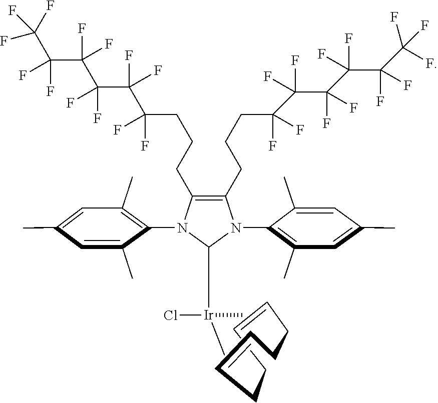

Potassium tert-butoxide (112 mg, 1.00 mmol, 2.5 eq.) was added to a stirred solution of 1,3-dimesityl-4,5-bis(4,4,5,5,6,6,7,7,8,8,9,9,9-tridecafluorononyl)-1H-imidazol-3-ium chloride (100 mg, 94.2 μmol, 2.2 eq) from Example 29 in tetrahydrofuran (10 mL) at room temperature in a glove box. The resulting suspension was stirred for 30 minutes. A solution of [Ir(COD)C_{1]}₂ (28.8 mg, 42.8 μmol, 1.0 eq.) was added and the resulting solution was stirred at room temperature overnight (Cowley et al., J. Am. Chem. Soc., 133, 6134-6137 (2011)). The solvent was removed under reduced pressure to give the crude product and dried overnight under vacuum. The solvent was removed under reduced pressure to give the crude product and dried overnight under vacuum. This sample was purified with flash chromatography DCM/hexane (4:1). MS (ESI) m/z (%): 1325 [M-Cl]⁺(100).

### Example 31

This example illustrates a method of hyperpolarizing a [1-¹³C]pyruvate in a mixture of deuterated or non-deuterated methanol and fluorinated solvent (Novec™ 7100 fluid) in accordance with an aspect of the invention, as shown in Scheme 6.

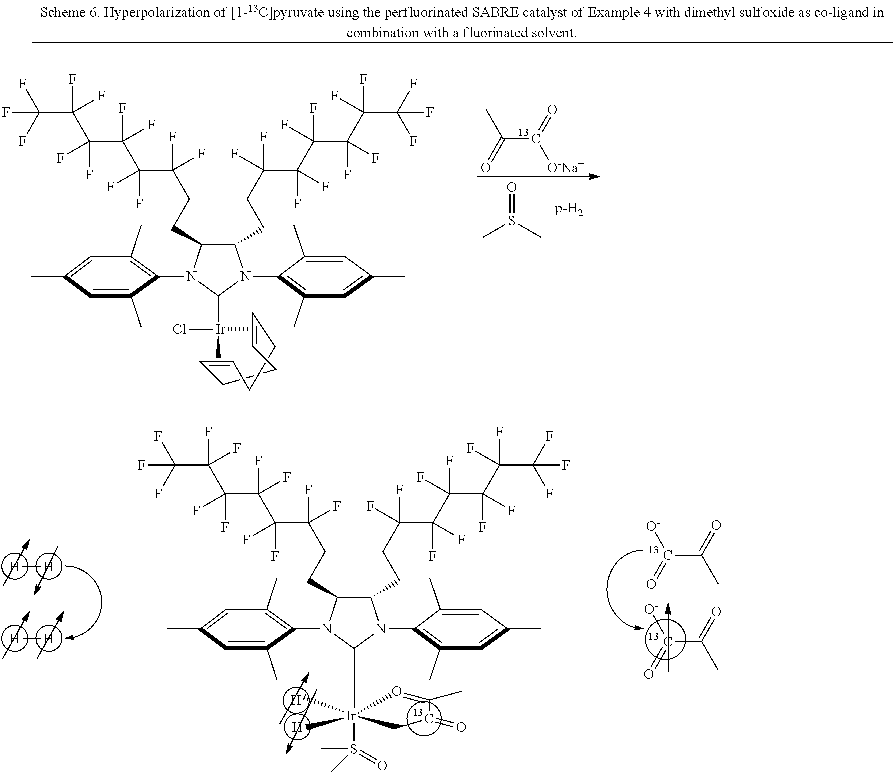

In the reaction scheme above, hyperpolarization of [1-¹³C]pyruvate was performed using SABRE in SHield Enables Alignment Transfer to Heteronuclei (SABRE-SHEATH) (Theis et al., J Am. Chem. Soc., 137, 1404-1407 (2015) and Truong et al., J Phys. Chem. C, 119, 8786-8797 (2015)) tailored for the ¹³C nucleus (Barskiy et al., ChemPhysChem, 18, 1493-1498 (2017)) using the co-ligand approach developed by Duckett and co-workers (Iali et al., Angew. Chemie —Int. Ed., 58, 10271-10275 (2019)) in combination with a fluorinated solvent. Sodium [1-¹³C]-pyruvate and deuterated methanol-d₄ solvent were purchased from Sigma-Aldrich and the non-deuterated solvent 7100 fluid was purchased from Novec™. The compounds were used after being extensively purged from oxygen by bubbling Argon for 10 minutes and then further degassed by 3 cycles of freeze-thaw (FTC). The [IrCl(COD)(F-IMes)] SABRE catalyst precursor was synthesized from F-IMes, which was prepared according to Example 4. The active catalyst was prepared with a substrate [1-¹³C]-pyruvate in 0.2 mL of methanol-d₄, Ir(F-IMes) SABRE pre-catalyst in 0.3 mL Novec™ 7100 fluid, and a dimethyl sulfoxide (DMSO) co-ligand in a 5 mm NMR tube.

The method of hyperpolarizing set forth in Example 6 was repeated except where indicated below. In particular, the hyperpolarization transfer from p-H₂-derived Iridium hydrides to the ¹³C nuclear spin of [1-¹³C]pyruvate was attained by performing SABRE in sub-microtesla magnetic fields using SABRE in SHield Enables Alignment Transfer to Heteronuclei (SABRE-SHEATH) using a solution mixture of [IrCl(H)₂(DMSO)₂(F-IMes)], [1-¹³C]pyruvate, co-ligand (dimethyl sulfoxide) and p-H₂ in a mixture of deuterated or non-deuterated methanol and Novec™ 7100 fluid.

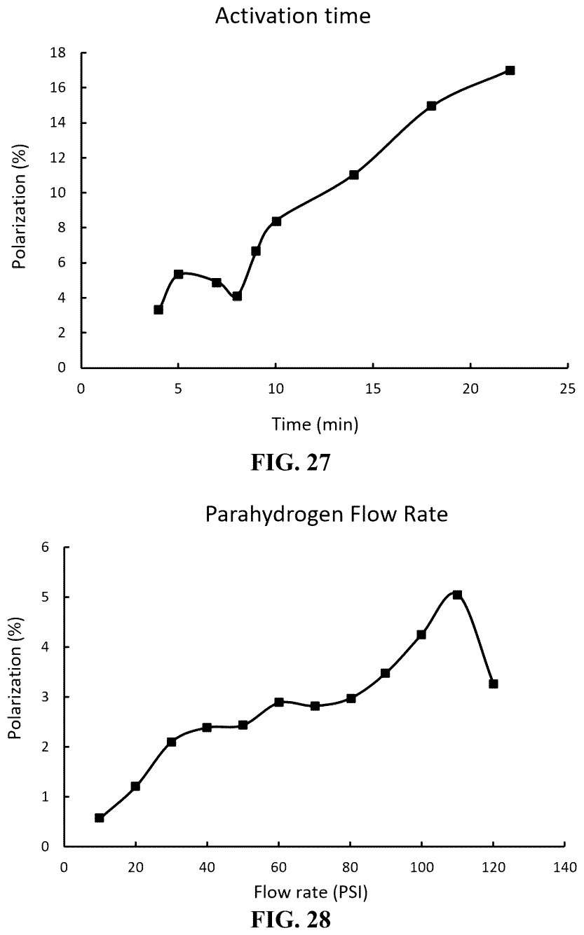

The fluorinated SABRE catalyst activation took less than 15 minutes, with the ¹³C polarization percentage shown in FIG. 27, was performed by bubbling ˜95% p-H₂ at a flow rate of 20 standard cubic centimeters per minute (scc/m) at 8 atm p-H₂ partial pressure, which leads to the formation of Complex 2, Complex 3a, Complex 3b, and pyruvate, as depicted in FIG. 8, in accord with the notation introduced by Duckett and co-workers (Iali et al., Angew. Chemie —Int. Ed., 58, 10271-10275 (2019)). The NMR samples consisted in about −25 mM sodium [1-¹³C]pyruvate in 0.2 mL CD₃OD, 7.4 mM fluorinated SABRE catalyst, and 40 mM dimethyl sulfoxide (DMSO) in 0.3 mL Novec™ 7100, the mixing field was at 0.4 μT and temperature at 0° C. Without wishing to be bound by any particular theory, it is believed that Complex 3B is the primary SABRE-active species.

Optimization of p-H₂ flow rate was evaluated and is presented in FIG. 28. The NMR samples consisted in 35 mM sodium [1-¹³C]pyruvate in 0.3 mL CD₃OD, 6 mM fluorinated SABRE catalyst, and 112 mM dimethyl sulfoxide (DMSO) in 0.2 mL Novec™ 7100 fluid, the mixing field was at 0.4 μT, and temperature at 0° C. When the flow rate exceeded 120 PSI, the solution developed excessive bubbling akin to a soapy solution, rendering it impractical to consistently measure polarization.

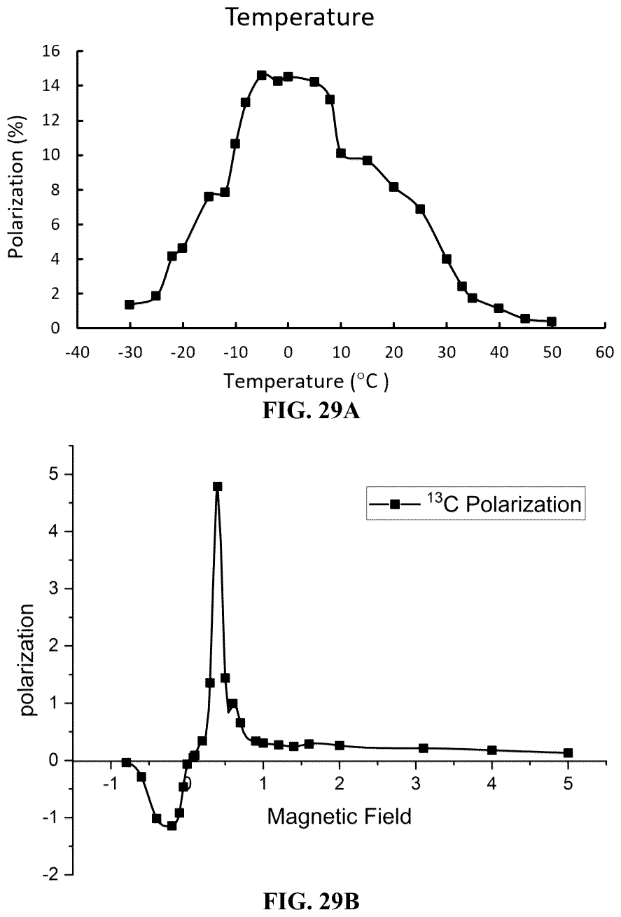

Optimization of the temperature and magnetic field in micro-Tesla regime were evaluated and the results are set forth in FIGS. 29A and 29B. The NMR samples consisted in 35 mM sodium [1-¹³C]pyruvate, 6 mM fluorinated SABRE catalyst in 0.3 mL CD₃OD, and 56 mM dimethyl sulfoxide (DMSO) in 0.2 mL Novec™ 7100 fluid, p-H₂ flow and pressure 90 ssc/m and 100 PSI, respectively. The optimum temperature was reacted at 0° C. and the mixing field at 0.4 μT.

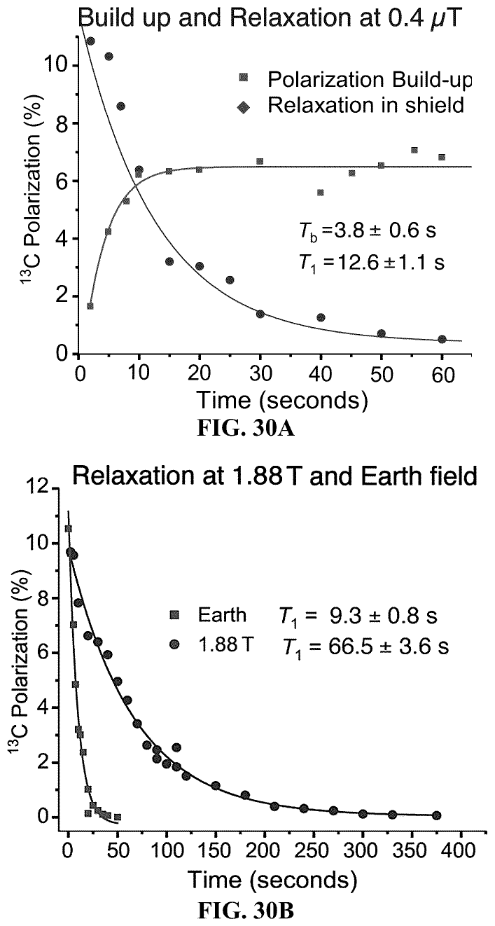

The relaxation dynamics are set forth in FIGS. 30A and 30B. As is apparent from the results set forth in FIGS. 30A and 30B, the relaxation dynamics of [1-¹³C]-pyruvate showed that the total P_{13C} (bound+free) build-up time (T_{b}=3.8±0.6 s) was shorter that the corresponding T₁ value of 12.6±1.1 s, which allowed to reach P_{13C} levels up to 6.02%. In addition, relaxation dynamics at earth field and 1.8T were evaluated at T₁=9.3±0.8 s and 66.5±3.6 s, respectively.

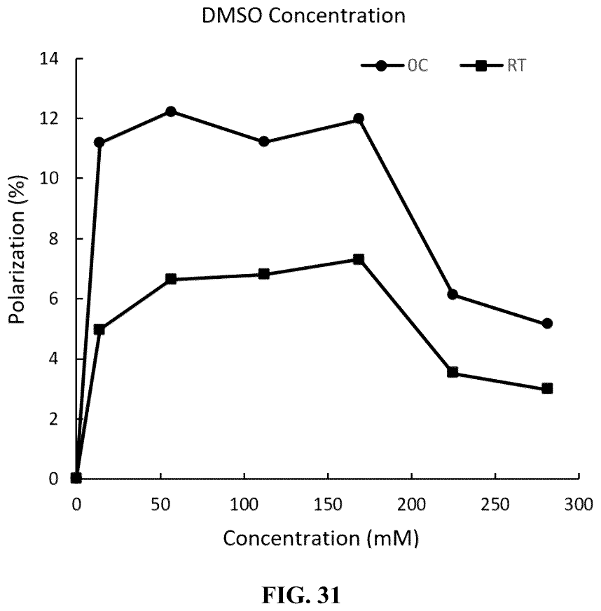

Optimization of the co-ligand (i.e., dimethyl sulfoxide (DMSO)) concentration was evaluated and the results are set forth in FIG. 31. The NMR sample consisted in 35 mM sodium [1-¹³C]pyruvate in 0.3 mL CD₃OD, and 6 mM fluorinated SABRE catalyst in 0.2 mL Novec™ 7100 fluid, p-H₂ flow and pressure 90 ssc/m and 100 PSI, respectively. The SABRE-SHEATH polarization was performed at 0° C. (i.e., OC) and room temperature (i.e., RT). The mixing field was fixed at 0.4 μT.

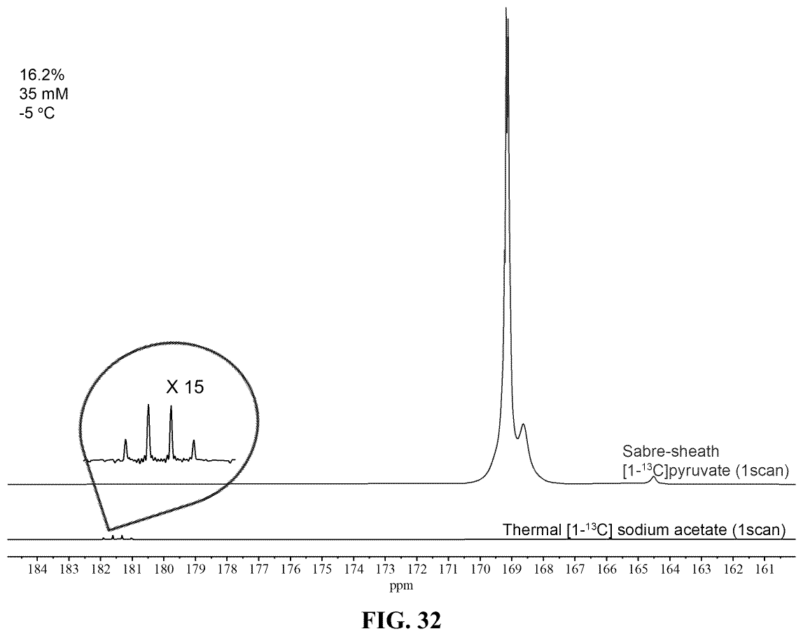

The exchange of p-H₂ and [1-¹³C]pyruvate on activated Ir F-IMes catalyst leads to buildup of ¹³C hyperpolarization, as shown in FIG. 32. In that respect, FIG. 32 provides a representative spectrum of ¹³C-hyperpolarized [1-¹³C]-pyruvate with signal enhancement ε of ˜104010 fold, corresponding to P_{13C} of ˜16.2% obtained via comparison of the NMR signal intensity with a reference sample. The NMR samples consisted in 35 mM sodium [1-¹³C]pyruvate in 0.3 mL CD₃OD, 6 mM fluorinated SABRE catalyst, and 56 mM dimethyl sulfoxide (DMSO) in 0.2 mL Novec™ 7100 fluid, the mixing field was at 0.4 μT and temperature at −5° C. with a 85% enriched parahydrogen pressure and flow at 100 PSI and 90 scc/m, respectively.

This example shows that the hyperpolarization of SABRE can be improved with fluorous solvents such as Novec™ 7100 fluid. Without wishing to be bound by any particular theory, it is believed that the increase in polarization may be due to improved parahydrogen hydrogen solubility in the fluorous solvent.

### Example 32

This example illustrates a method of hyperpolarizing a [1-¹³C]pyruvate in a mixture of deuterated or non-deuterated methanol and fluorinated solvent (Novec 7100 fluid) in combination with phenyl trifluoromethyl sulfoxide as a co-ligand in accordance with an aspect of the invention, as shown in Scheme 7.

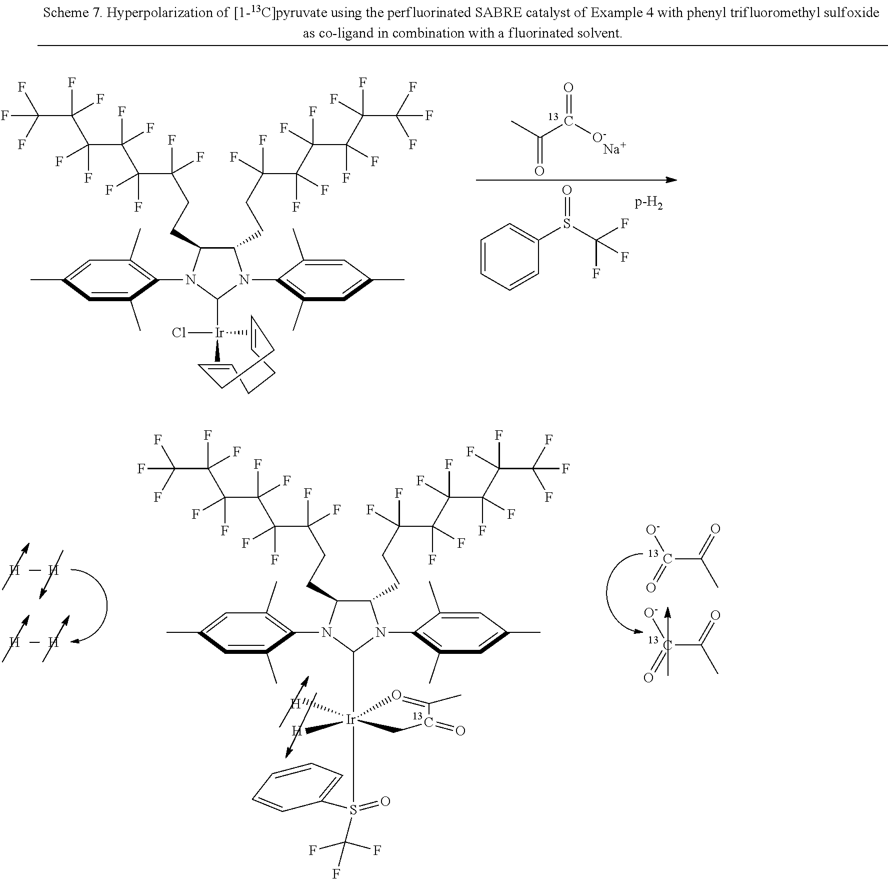

In the reaction scheme above, hyperpolarization of [1-13C]pyruvate was performed using SABRE in SHield Enables Alignment Transfer to Heteronuclei (SABRE-SHEATH) (Theis et al., J. Am. Chem. Soc., 137, 1404-1407 (2015) and Truong et al., J. Phys. Chem. C, 119, 8786-8797 (2015)) tailored for the 13C nucleus (Barskiy et al., ChemPhysChem, 18, 1493-1498 (2017)) using the co-ligand approach developed by Duckett and co-workers (Iali et al., Angew. Chemie —Int. Ed., 58, 10271-10275 (2019)) in combination with a fluorinated solvent. Sodium [1-13C]-pyruvate and deuterated methanol-d₄ solvent were purchased from Sigma-Aldrich and the non-deuterated solvent 7100 fluid was purchased from Novec™. The compounds were used after being extensively purged from oxygen by 3 cycles of freeze-thaw (FTC) and then further degassed by bubbling Argon for 10 minutes. The [IrCl(COD)(F-IMes)] SABRE catalyst precursor was synthesized from F-IMes, which was prepared according to Example 4. The active catalyst was prepared with a substrate [1-13C]-pyruvate in 0.2 mL of methanol-d₄, Ir(F-IMes) SABRE pre-catalyst in 0.3 mL Novec™ 710fid and a phenyl trifluoromethyl sulfoxide (PTMSO) co-ligand in a 5 mm NMR tube. The method of hyperpolarizing set forth in Example 6 was repeated except where indicated below. In particular, the hyperpolarization transfer from p-H₂-derived Iridium hydrides to the ¹³C nuclear spin of [1_13C]pyruvate was attained by performing SABRE in sub-microtesla magnetic fields using SABRE in SHield Enables Alignment Transfer to Heteronuclei (SABRE-SHEATH) using a solution mixture of [IrCl(H)₂(PTMSO)₂(F-IMes)], [1-¹³C]pyruvate, co-ligand (phenyl trifluoromethyl sulfoxide) and p-H₂ in a mixture of deuterated or non-deuterated methanol and Novec™ 7100 fluid.

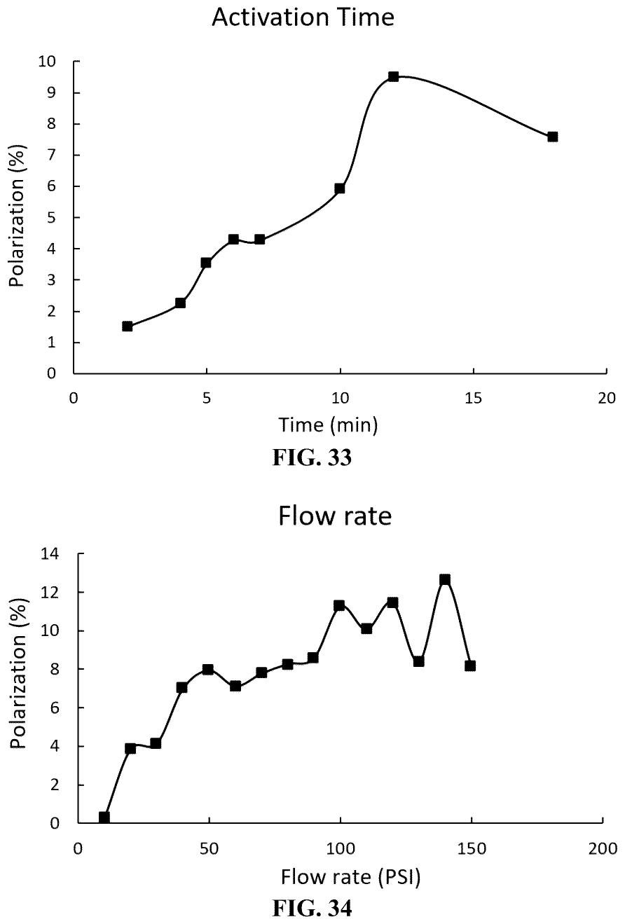

The fluorinated SABRE catalyst activation took less than 15 minutes, with the ¹³C polarization percentage shown in FIG. 33, was performed by bubbling ˜95% p-H₂ at a flow rate of 20 standard cubic centimeters per minute (scc/m) at 8 atm p-H₂ partial pressure, which leads to the formation of Complex 2, Complex 3a, Complex 3b, and pyruvate, as depicted in FIG. 8, in accord with the notation introduced by Duckett and co-workers (Iali et al., Angew. Chemie —Int. Ed., 58, 10271-10275 (2019)). The NMR samples consisted in about 35 mM sodium [1-¹³C]pyruvate in 0.2 mL CD₃OD, 6 mM fluorinated SABRE catalyst, and −117 mM phenyl trifluoromethyl sulfoxide (PTMSO) in 0.3 mL Novec™ 7100, the mixing field was at 0.4 μT, and room temperature. Without wishing to be bound by any particular theory, it is believed that Complex 3B is the primary SABRE-active species.

Optimization of p-H₂ flow rate was evaluated and is presented in FIG. 34. The NMR samples consisted in 35 mM sodium [1-¹³C]pyruvate in 0.3 mL CD₃OD, 6 mM fluorinated SABRE catalyst, and 175 mM phenyl trifluoromethyl sulfoxide (PTMSO) in 0.2 mL Novec™ 7100 fluid, the mixing field was at 0.4 μT, and room temperature. Similarly as described in Example 31, when the flow rate exceeded 120 PSI, the solution developed excessive bubbling akin to a soapy solution, rendering it impractical to consistently measure polarization.

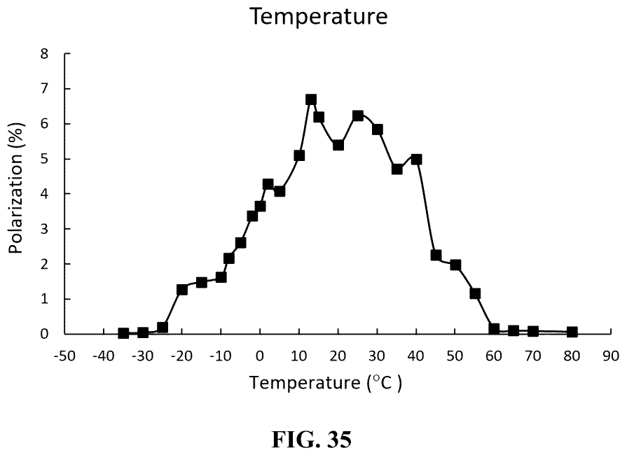

Optimization of the temperature and was evaluated and the results are set forth in FIG. 35. The NMR sample consisted in 35 mM sodium [1-¹³C]pyruvate, 6 mM fluorinated SABRE catalyst in 0.3 mL CD₃OD, and 117 mM phenyl trifluoromethyl sulfoxide (PTMSO) in 0.2 mL Novec™ 7100 fluid, p-H₂ flow and pressure 90 ssc/m and 100 PSI, respectively. The optimum temperature is reach at 13° C. and the mixing field at 0.4 PT.

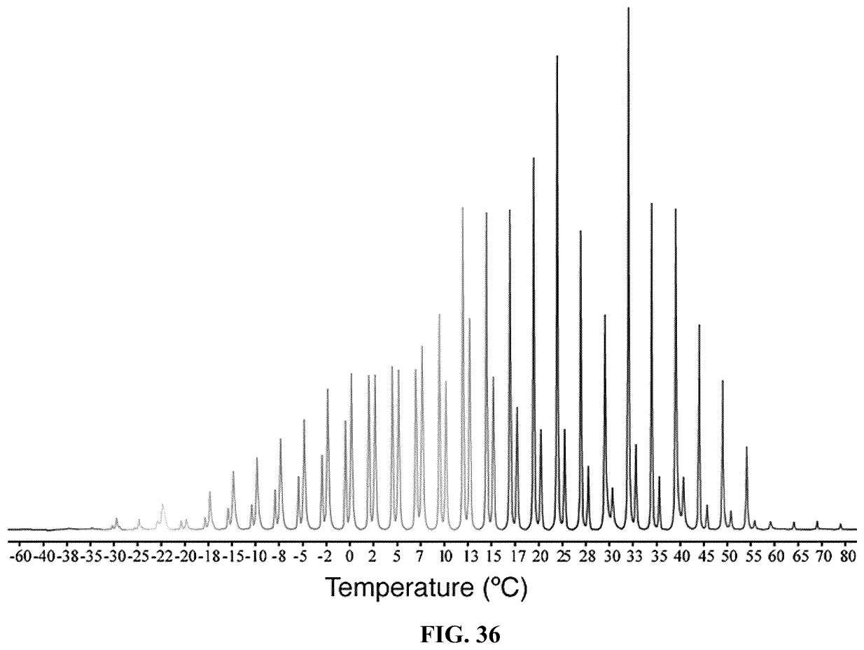

The temperature sweep provided in FIG. 36 defines the distribution among the four main species, which is similar to [IrCl(COD)(IMes)] (Adelabu et al., ChemPhysChem, 23, e202100839 (2022)) and different from the DMSO-based Ir F-IMes of Example 31. The exchange rates of Ir F-IMes and the original Ir Mes SABRE catalyst are very comparable, and these exchange rates are believed to facilitate most of the accumulation of the hyperpolarized free pyruvate's NMR signal within the time scale of the SABRE experiment. Without wishing to be bound by any particular theory, it is believed that the SABRE process yields substantial signal enhancement only if the substrate chemical exchange process is sufficiently fast. Conversely, a slow exchange rate prevents a bulk polarization to build up in the free molecule, leading to decay attributed to relaxation processes. See, for example, Shchepin et al. [J. Phys. Chem. Lett., 6 (10), 1961-1967 (2015)], Iali et al. [Chem. Sci., 9 (15), 3677-3684 (2018)], and Lloyd et al. [Catal. Sci. Technol., 4 (10), 3544-3554 (2014)]. The optimum polarization transfer temperature for hyperpolarized pyruvate with the PTMSO-based Ir F-IMes catalyst is found at TT of 13° C., which is similar than that of the original IMes SABRE catalyst.

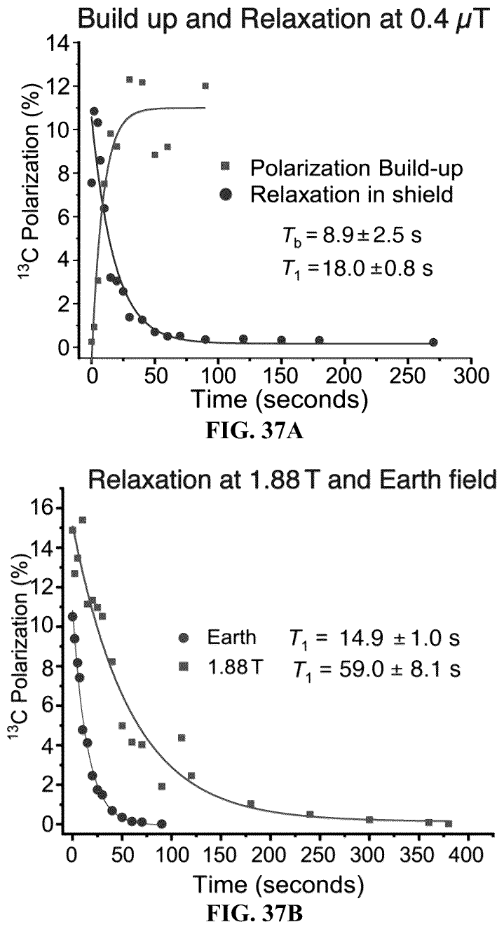

The relaxation dynamics are set forth in FIGS. 37A and 37B. As is apparent from the results set forth in FIGS. 37A and 37B, the relaxation dynamics of [1-¹³C]-pyruvate showed that the total P_{13C} (bound+free) build-up time (T_{b}=8.9±2.5 s) was substantially shorter that the corresponding T₁ value of 18.0±0.8 s, which allowed to reach P_{13C} levels up to 6.02%. In addition, relaxation dynamics at earth field and 1.8T were evaluated at T₁=14.9±1.0 s and 59.0±8.1 s, respectively.

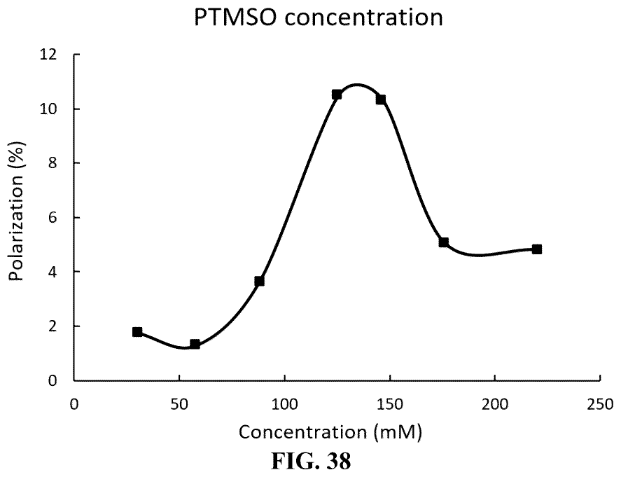

Optimization of the co-ligand (i.e., phenyl trifluoromethyl sulfoxide (PTMSO)) concentration was evaluated and the results are set forth in FIG. 38. The NMR sample consisted in 35 mM sodium [1-¹³C]pyruvate in 0.3 mL CD₃OD, and 6 mM fluorinated SABRE catalyst in 0.2 mL Novec™ 7100 fluid, p-H₂ flow and pressure 90 ssc/m and 100 PSI, respectively. The SABRE-SHEATH polarization was performed at room temperature. The mixing field was fixed at 0.4 μT.

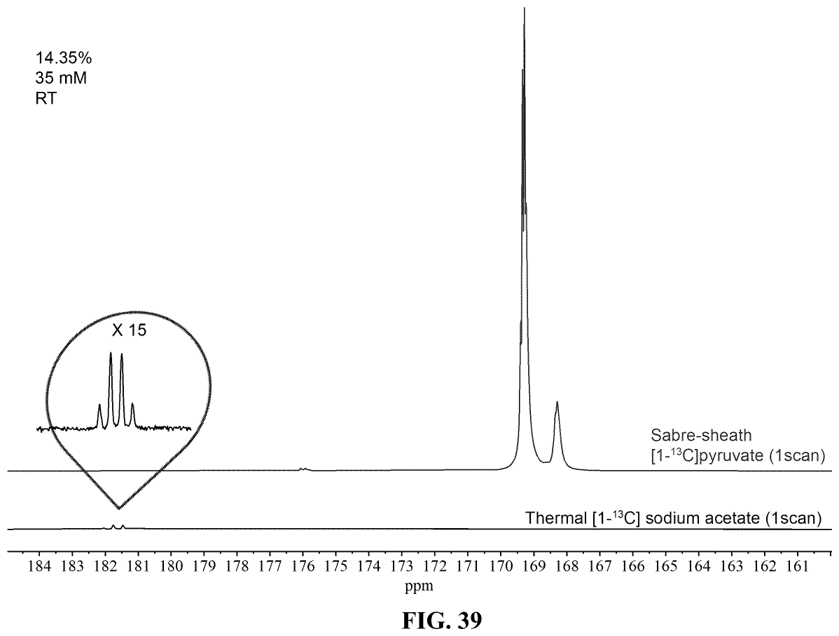

The exchange of p-H₂ and [1-¹³C]pyruvate on activated Ir F-IMes catalyst leads to buildup of ¹³C hyperpolarization, as shown in FIG. 39. In that respect, FIG. 39 provides a representative spectrum of ¹³C-hyperpolarized [1-¹³C]-pyruvate with signal enhancement ε of ˜92018 fold, corresponding to P_{13C} of ˜14.35% obtained via comparison of the NMR signal intensity with a reference sample. The NMR samples consisted in 35 mM sodium [1-¹³C]pyruvate in 0.3 mL CD₃OD, 6 mM fluorinated SABRE catalyst, and 117 mM phenyl trifluoromethyl sulfoxide (PTMSO) in 0.2 mL Novec™ 7100 fluid, the mixing field was at 0.4 μT and temperature at room temperature ° C. with a 85% enriched parahydrogen pressure and flow at 100 PSI and 90 scc/m, respectively.

The PTMSO-based Ir F-IMes catalyst of this example increased solubility of the perfluorinated SABRE catalyst in fluorinated solvent leading to a reduction in the iridium content in the aqueous phase. Without wishing to be bound by any particular theory, it is believed that other fluorinated DMSO derivatives such as 2,2,2-trifluoroethyl methane sulfonate or di-2,2,2-trifluoroethyl sulfoxide, for example, could provide beneficial properties such as reduced iridium content in the aqueous phase.

### Example 33

This example illustrates an exemplary method for isolating hyperpolarized sodium [1-¹³C]pyruvate using DMSO as a co-ligand, which includes extraction and filtration.

Hyperpolarization of sodium [1-¹³C]pyruvate was performed using SABRE in SHield Enables Alignment Transfer to Heteronuclei (SABRE-SHEATH) tailored for the ¹³C nucleus using dimethyl sulfoxide (DMSO) as a co-ligand, [IrCl(COD)(F-IMes)] SABRE catalyst precursor, and parahydrogen enriched to about 70 to 95% in deuterated methanol-d₄ solvent and Novec™ 7100, as described in Examples 31 and 32. The optimum parameters described previously in Examples 31 and 32, such as temperature, transfer field, para-hydrogen flow, and pressure, were used in this example. The SABRE samples consisted in 35 mM sodium [1-¹³C]pyruvate in 0.3 mL CD₃OD, 6 mM fluorinated [IrCl(COD)(F-IMes)] SABRE catalyst precursor, which was prepared according to Example 4, and 56 mM dimethyl sulfoxide (DMSO) in 0.2 mL Novec™ 7100, the para-hydrogen flow was established at 90 ss/m and pressurized at 8 bars, and the mixing field was at 0.4 μT and a temperature of 0° C.

After the hyperpolarization procedure was completed, the pyruvate hyperpolarization was measured at 13.6%, and the sample was rapidly removed from the 0.40 μT field and depressurized. Heavy water (D₂O) (200 μL) was added to the mixture, thereby resulting in the formation of an emulsion. The hyperpolarized pyruvate in solution was measure at 5.9%. The emulsion was transferred to a 1 mL plastic syringe held mounted to a Luer-locked filter (Waters Oasis Prime HLB Plus Light Cartridge (Part Number: 186008866) in order to break the emulsion. The aqueous solution was guided through the filter into a 5 mm NMR tube, already located into the adjacent 1.8 T benchtop NMR spectrometer, and the pyruvate hyperpolarization was measured at 0.34%. The whole procedure took about 1.30 minutes.

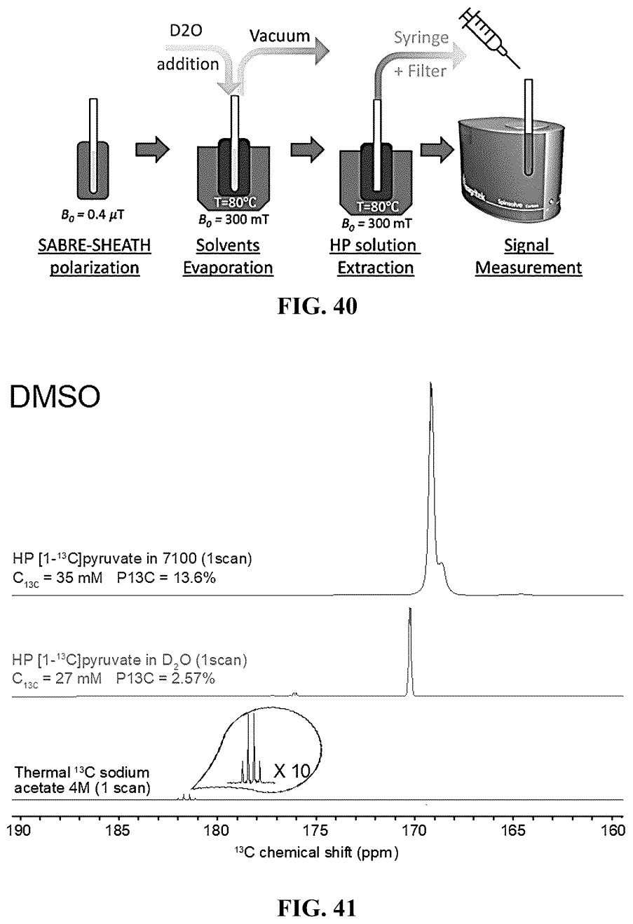

Inspired by these results, an alternative approach was explored which involved the rapid evaporation of methanol [Ding et al. (Chemistry-Methods, 2 (7), (2022)) and de Maissin et al. (Chemie Int. Ed., 62 (36), (2023))], as depicted in FIG. 40. This approach consisted of adding heavy water (D₂O) to the hyperpolarized SABRE sample in an NMR tube and subsequently placing the sample in a hot water bath while applying a vacuum (≈1 mbar) in order to evaporate the methanol and Novec™ 7100 (methanol and water do not form an azeotrope, and therefore, they evaporate at distinct boiling points). The sample was evacuated for 5 seconds at 80° C., resulting in the evaporation of the methanol and the Novec™ 7100. The final methanol concentration dropped from 12.36 M to 6.0 M and retained 35 mM of the pyruvate in the resulting ≈500 μL aqueous solution. Extended evacuation can be applied to increase the pyruvate concentration. Finally, the hyperpolarized pyruvate in solution was transferred to a 1 mL plastic syringe held mounted to a Luer-locked filter (Waters Oasis Prime HLB Plus Light Cartridge (Part Number: 186008866) and guided through the filter into a 5 mm NMR tube, which was already located in the adjacent 1.8 T benchtop NMR spectrometer. The results are set forth in FIG. 41. The pyruvate hyperpolarization was measured at 2.57%, down from an initial level of 13.6%. The whole procedure took about 2 minutes. In addition, the concentration of pyruvate, methanol, DMSO, and Novec™ 7100 in five separate samples was measured by LC-MS (liquid chromatography mass spectrometry) and iridium concentration was measured by ICP-MS (inductively coupled plasma mass spectrometry). The results are set forth in Table 1.

TABLE 1
LC-MS and ICP-MS Results for Example 33

| | Pyruvate | Methanol | DMSO | Novec ™ 7100 | Iridium |
|---|---|---|---|---|---|
| | conc. (mM) | conc. (mM) | conc. (mM) | conc. (mM) | conc. (ppb) |
| Sample 1 | 35.8 ± 6.75 | 10713 ± 1562.6 | 0.45 ± 0.061 | 0.123 ± 0.0335 | 596 |
| Sample 2 | 26.4 ± 1.23 | 6093 ± 433.8 | 3.17 ± 0.457 | 0.194 ± 0.0358 | 277 |
| Sample 3 | 27.7 ± 9.5 | 6537 ± 1009 | 3.52 ± 0.784 | 0.154 ± 0.0359 | — |
| Sample 4 | 25.0 ± 1.24 | 8013 ± 789.5 | 2.04 ± 0.236 | 0.174 ± 0.0205 | 310 |
| Sample 5 | 28.3 ± 2.9 | 9413 ± 1686.5 | 1.55 ± 0.151 | 0.157 ± 0.0497 | — |
| Sample 6 | 38.4 ± 2.56 | 7682 ± 528.2 | 3.51 ± 0.643 | 0.16 ± 0.046 | 683 |
| Sample 7 | 24.4 ± 9.12 | 5669 ± 605.2 | 4.29 ± 0.453 | 0.155 ± 0.05 | 417 |
| Sample 8 | 20.36 ± 1.49 | 6260 ± 463.8 | 2.74 ± 0.020 | 0.147 ± 0.013 | 705 |
| Sample 9 | 26.03 ± 1.45 | 7951 ± 501.4 | 1.64 ± 0.161 | 0.128 ± 0.037 | 93 |
| Sample 10 | 30.5 ± 1.95 | 10875 ± 671 | 1.44 ± 0.057 | 0.1853 ± 0.048 | 161 |

Note that samples 9 and 10 contained 1 mM of EDTA (ethylenediaminetetraacetic acid).

As is apparent from the results set forth in Table 1, between 57 and 100% of the sodium [1-¹³C]pyruvate concentration was collected after filtration.

### Example 34

This example illustrates an exemplary method for isolating hyperpolarized sodium [1-¹³C]pyruvate using phenyl trifluoromethyl sulfoxide (PTMSO) as a co-ligand, which includes extraction and filtration.

Hyperpolarization of sodium [1-¹³C]pyruvate was performed using SABRE in SHield Enables Alignment Transfer to Heteronuclei (SABRE-SHEATH) tailored for the ¹³C nucleus using phenyl trifluoromethyl sulfoxide (PTMSO) as a co-ligand, [IrCl(COD)(F-IMes)] SABRE catalyst precursor, and parahydrogen enriched to about 70 to 95% in deuterated methanol-d₄ solvent and Novec™ 7100, as described in Examples 31 and 32. The optimum parameters described previously in Examples 31 and 32, such as temperature, transfer field, para-hydrogen flow, and pressure, were used in this example. The SABRE samples consisted in 35 mM sodium [1-¹³C]pyruvate in 0.3 mL CD₃OD, 6 mM fluorinated [IrCl(COD)(F-IMes)] SABRE catalyst precursor, which was prepared according to Example 4, and 56 mM phenyl trifluoromethyl sulfoxide (PTMSO) in 0.2 mL Novec™ 7100, the para-hydrogen flow was established at 90 ss/m and pressurized at 8 bars, and the mixing field was at 0.4 μT and a temperature of 0° C.

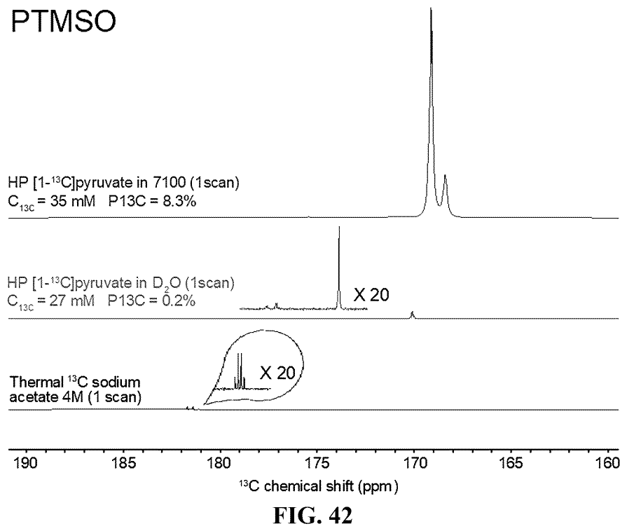

The alternative approach of Example 33, which included the rapid evaporation of methanol, was performed and the results are set forth in FIG. 42. The pyruvate hyperpolarization was measured at 0.2%, down from an initial level of 8.3%. The whole procedure took about 2 minutes. In addition, the concentration of pyruvate, methanol, PTMSO, and Novec™ 7100 in two separate samples was measured by LC-MS and iridium concentration was measured by ICP-MS. The results are set forth in Table 2.

TABLE 2
LC-MS and ICP-MS Results for Example 34

| | Pyruvate | Methanol | PTMSO | Novec ™ 7100 | Iridium |
|---|---|---|---|---|---|
| | conc. (mM) | conc. (mM) | conc. (mM) | conc. (mM) | conc. (ppb) |
| Sample 1 | 20.1 ± 6.76 | 7975 ± 2312.5 | 0.02 ± 0.018 | 0.133 ± 0.0619 | — |
| Sample 2 | 25.1 ± 1.30 | 14443 ± 873.1 | 0.01 ± 0.002 | 0.177 ± 0.0797 | — |
| Sample 3 | 12.1 ± 2.69 | 5057.9 ± 325.1 | 0.008 ± 0.002 | 0.133 ± 0.06 | 217 |
| Sample 4 | 25.1 ± 1.28 | 10666 ± 455 | 0.006 ± 0.001 | 0.176 ± 0.079 | 74 |

As is apparent from the results set forth in Table 2, between 57 and 71% of the sodium [1-¹³C]pyruvate concentration was collected after filtration.

### Example 35

This example illustrates an exemplary method for isolating hyperpolarized sodium [1-¹³C]pyruvate, which includes an extraction with an aqueous phase and a fluorinated phase.

Hyperpolarization of sodium [1-¹³C]pyruvate was performed using SABRE in SHield Enables Alignment Transfer to Heteronuclei (SABRE-SHEATH) tailored for the ¹³C nucleus using dimethyl sulfoxide (DMSO) or phenyl trifluoromethyl sulfoxide (PTMSO) as a co-ligand, [IrCl(COD)(F-IMes)] SABRE catalyst precursor, and parahydrogen enriched to about 70 to 95% in deuterated methanol-d₄ solvent and Novec™ 7100, as described in Examples 31 and 32. The optimum parameters described previously in Examples 31 and 32, such as temperature, transfer field, para-hydrogen flow, and pressure, were used in this example. The SABRE samples were prepared in a 9 inch NMR tube with 30 mM sodium [1-¹³C]pyruvate in 0.3 mL CD₃OD, 5 mM fluorinated [IrCl(COD)(F-IMes)] SABRE catalyst precursor, which was prepared according to Example 4, and 25 mM dimethyl sulfoxide (DMSO) or 98 mM phenyl trifluoromethyl sulfoxide (PTMSO) in 0.3 mL Novec™ 7100, the para-hydrogen flow was pressurized at 8 bars, the para-hydrogen flow was set at 20 ss/m for activation and then at 90 ss/m for hyperpolarization, and the mixing field was at 0.4 μT and a temperature of 0° C. for DMSO or room temperature for PTMSO.

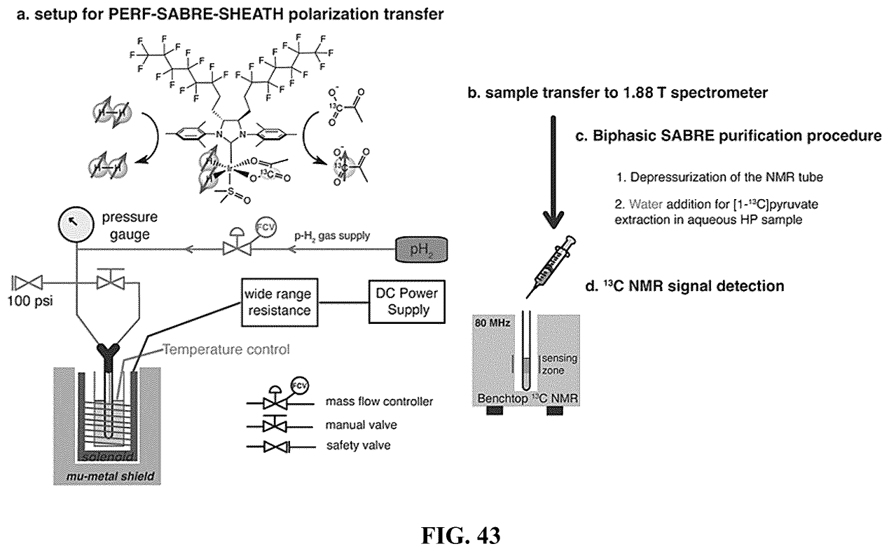

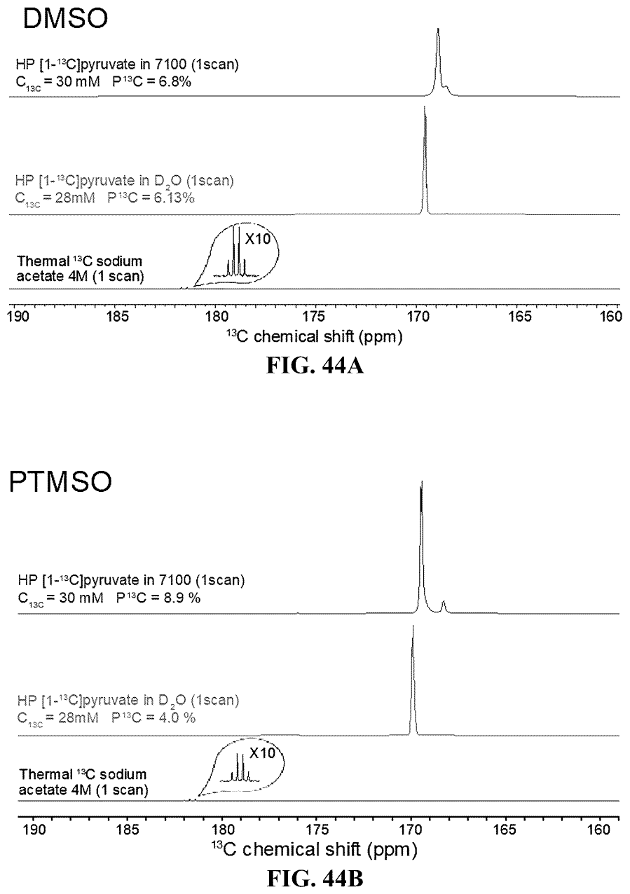

After the hyperpolarization procedure was completed, the sample was rapidly removed from the 0.4 μT field and transferred inside the NMR spectrometer at 1.8 T at room temperature. The NMR sample was depressurized and 300 μL of heavy water (D₂O) was added to the solution in order to drive the hyperpolarized sodium [1-¹³C]pyruvate into the aqueous phase and keep the perfluorinated SABRE catalyst in the organic fluorinated phase. The procedure is depicted in FIG. 43. Since the water phase is located above the spectrometer sensing zone, the 9-inch NMR tube is adjusted deeper into the spectrometer to facilitate the measurement of the pyruvate signal in the aqueous phase. The separation between the organic and aqueous phase was well defined. The results for DMSO and PTMSO are set forth in FIGS. 44A and 44B, respectively.

Signal enhancement in the water phase was measured at ε˜39300 fold and ˜25700 fold, corresponding to P_{13C} of ˜6.13% (down from its initial level of 6.8%) and 4% (down from its initial level of 8.9%), for DMSO and PTMSO, respectively. Those values were obtained via comparison of the NMR signal intensity, as shown in FIGS. 44A and 44B, with a reference sample. When DMSO was employed as the co-ligand, the presence of a residual pyruvate bound signal can be observed, indicating that a portion of the complex remains in the water phase. The ratio of methanol to the fluorinated solvent plays a vital role in achieving effective phase separation. If an insufficient amount of fluorinated solvent is utilized, an emulsion may form when water is added. In addition, the use in exces of DMSO, which is miscible in water, may contribute to the partial solubility of the complex in the aqueous phase, unlike the phenyl methyltrifluoro sulfoxide which is insoluble in methanol.

The aqueous phase containing the sodium [1-¹³C]pyruvate was evacuated and tested for iridium content (ICP-MS) and pyruvate concentration (LC-MS). The ICP-MS results showed a content of between 310 and 786 ppb iridium in all samples tested. The LC-MS and ICP-MS results for the concentration of pyruvate, methanol, DMSO or PTMSO, and Novec™ 7100, and iridium content in six separate samples are set forth in Table 3.

TABLE 3
LC-MS and ICP-MS Results for Example 35

| | Pyruvate | Methanol | DMSO/PTMSO | Novec ™ 7100 | Iridium |
|---|---|---|---|---|---|
| | conc. (mM) | conc. (mM) | conc. (mM) | conc. (mM) | conc. (ppb) |
| Sample 1 (PTMSO) | 30.53 ± 1.79 | 13277 ± 496.2 | 6.42 ± 0.42 | 0.182 ± 0.040 | 433 |
| Sample 2 (PTMSO) | 20.06 ± 1.37 | 9076 ± 442.6 | 3.18 ± 0.38 | 0.154 ± 0.039 | 719 |
| Sample 3 (PTMSO) | 20.46 ± 2.09 | 7618 ± 1021 | 2.7 ± 0.049 | 0.158 ± 0.050 | 310 |
| Sample 4 (PTMSO) | 20.66 ± 1.09 | 14442 ± 873.06 | 5.06 ± 0.352 | 0.178 ± 0.069 | 383 |
| Sample 5 (DMSO) | 35.16 ± 1.95 | 14784.7 ± 1634 | 1.53 ± 0.202 | 0.16 ± 0.03 | 786 |
| Sample 6 (DMSO) | 24.86 ± 3.05 | 11827 ± 1352 | 1.57 ± 0.29 | 0.17 ± 0.11 | 560 |

As is apparent from the results set forth in Table 3, between 66 and 10000 of the sodium [1-¹³C]pyruvate concentration was collected after filtration, with 100% of the sodium [1-¹³C]pyruvate concentration being collected from the DMSO sample.

### Example 36

This example illustrates an exemplary method for isolating hyperpolarized sodium [1-¹³C]pyruvate, which includes recycling and reusing the SABRE catalyst.

Hyperpolarization of sodium [1-¹³C]pyruvate was performed using SABRE in SHield Enables Alignment Transfer to Heteronuclei (SABRE-SHEATH) tailored for the ¹³C nucleus using dimethyl sulfoxide (DMSO) or phenyl trifluoromethyl sulfoxide (PTMSO) as a co-ligand, [IrCl(COD)(F-IMes)] SABRE catalyst precursor, and parahydrogen enriched to about 70 to 95% in deuterated methanol-d₄ solvent and Novec™ 7100, as described in Examples 31 and 32. The optimum parameters described previously in Examples 31 and 32, such as temperature, transfer field, para-hydrogen flow, and pressure, were used in this example. The SABRE samples were prepared in a 9 inch NMR tube with 30 mM sodium [1-¹³C]pyruvate in 0.3 mL CD₃OD, 5 mM fluorinated [IrCl(COD)(F-IMes)] SABRE catalyst precursor, which was prepared according to Example 4, and 25 mM dimethyl sulfoxide (DMSO) or 98 mM phenyl trifluoromethyl sulfoxide (PTMSO) in 0.3 mL Novec™ 7100, the para-hydrogen flow was pressurized at 8 bars, the para-hydrogen flow was set at 20 ss/m for activation and then at 90 ss/m for hyperpolarization, and the mixing field was at 0.4 μT and a temperature of 0° C. for DMSO or room temperature for PTMSO.

After the hyperpolarization procedure was completed, the sample was rapidly removed from the 0.4 μT field and transferred inside the NMR spectrometer at 1.8 T at room temperature. The NMR sample was depressurized and 300 μL of heavy water (D₂O) was added to the solution in order to drive the hyperpolarized sodium [1-¹³C]pyruvate into the aqueous phase and keep the perfluorinated SABRE catalyst in the organic fluorinated phase, as described in Example 35.

Given the distinct separation between the fluorous organic and aqueous phases, the aqueous phase was removed. In the case of the reactions containing DMSO, the organic fluorinated portion collected, about 100 μL, represents a small portion of the original volume. Without wishing to be bound by any particular theory, it is believed that in the case of DMSO, most of the methanol fraction is driven into the aqueous phase along with the DMSO excess as it is miscible in both the fluorinated phase and the aqueous phase. In contrast, when using PTMSO as a co-ligand, most (about 250 μL) of the organic phase was recovered.

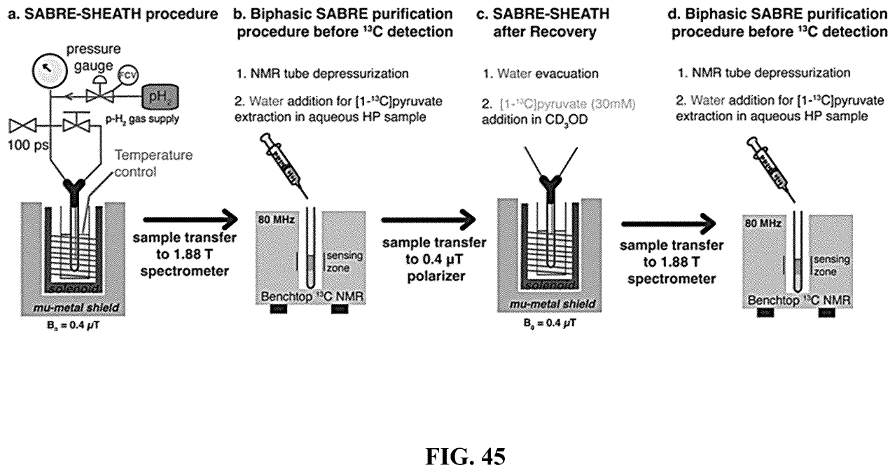

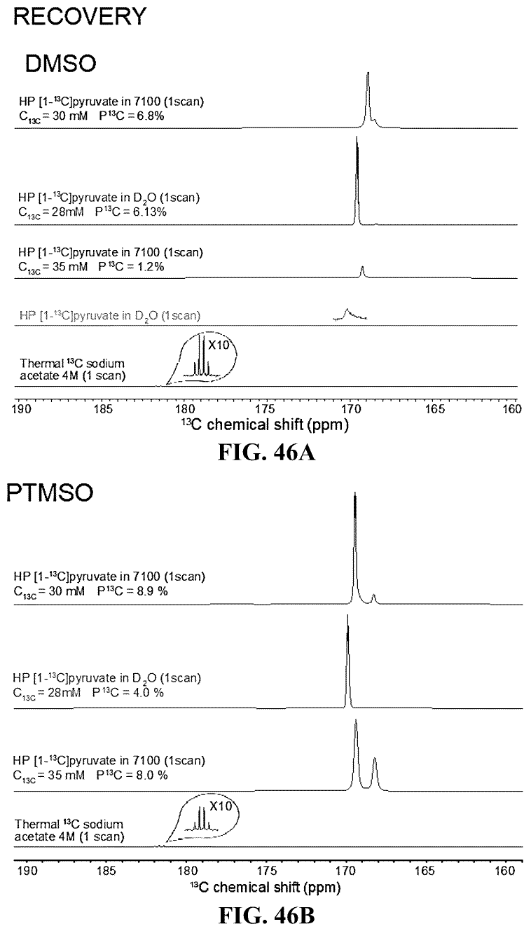

The perfluorinated catalyst in the fluorous organic phase was kept in the NMR tube and 300 μL deuterated methanol was added and contained 30 mM sodium [1-¹³C]pyruvate and the co-ligand being either 25 mM DMSO or 98 mM PTMSO. This mixture was then subjected to a second round of polarization in the SABRE-SHEATH polarizer. The procedure is depicted in FIGS. 45A-45D. The results for DMSO and PTMSO are set forth in FIGS. 46A and 46B, respectively. Signal enhancement in the water phase showed a polarization of the [1-¹³C] pyruvate with signal enhancement ε of ˜3090 fold, corresponding to P_{13C} of ˜1.2% (down from its initial level of 6.8%) for DMSO, and 8.0% (down from its initial level of 8.9%) for PTMSO. Those values were obtained via comparison of the NMR signal intensity with a reference sample.

The iterative process involving the hyperpolarization of [1-¹³C]pyruvate and its subsequent extraction in an aqueous phase, followed by the reutilization of the catalyst, was systematically conducted for a total of ten cycles. Impressively, no discernible diminishment in hyperpolarization activity was discerned after the second iteration of catalyst recycling. However, a noteworthy trend emerged during subsequent cycles. By the third recycle, a 39% diminution in [1-¹³C]pyruvate hyperpolarization in methanol was observed, marking the initial instance of a loss in polarization. Intriguingly, no further decline in hyperpolarization was noted until the sixth cycle, where a substantial 60.8% of the [1-¹³C]pyruvate polarization was lost.

Remarkably, the polarization remained relatively stable until the tenth cycle, which marked the conclusion of the experimental trials. It is imperative to highlight that the amount of co-ligand added in each cycle was not optimized, potentially contributing to the observed variations in hyperpolarization. Therefore, the potential for optimization persists, and the hyperpolarization for each cycle could be finely tuned by adjusting the precise amount of co-ligand added during the process.

Furthermore, the fluorinated catalyst exhibited a prolonged activity, retaining its efficacy for a duration of at least four days after the initial activation. This longevity suggests a robust and sustained catalytic performance. Consequently, this method presents a convenient and efficient approach, enabling the hyperpolarization of multiple batches of [1-¹³C]pyruvate using the same initial perfluorinated SABRE catalyst.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments can become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of preparing a hyperpolarized substrate, the method comprising:
(i) providing a perfluorinated SABRE catalyst comprising a d-block element and a perfluorinated ligand, wherein the perfluorinated ligand is of Formula (I):
[Lₘ-(NHC)—(Y—Z)_{q}] Formula (I),
or a salt thereof, and wherein
each L is independently selected from hydrogen, adamantyl, a substituted or unsubstituted aromatic group, or a substituted or unsubstituted heteroaromatic group,
NHC is a 4 to 7-membered N-heterocyclic carbenyl group where NHC is bound to the d-block element via a carbene,
each Y is independently selected from a bond or a spacer group,
each Z is a perfluorinated tag,
m is an integer from 1 to 4, and
q is an integer from 1 to 3;
(ii) providing a co-ligand to interact with the perfluorinated SABRE catalyst to facilitate formation of an active perfluorinated SABRE catalyst;
(iii) combining the active perfluorinated SABRE catalyst with parahydrogen and a substrate comprising a ½ spin nucleus or nuclei in a solvent to obtain a first reaction mixture;
(iv) hyperpolarizing the first reaction mixture obtained in (iii) by exposing the mixture to a magnetic field or by radiofrequency excitation to obtain a hyperpolarized active perfluorinated SABRE catalyst-substrate and/or a hyperpolarized substrate;
(v) performing an aqueous phase extraction and/or a fluorinated phase extraction to separate the hyperpolarized substrate from the perfluorinated SABRE catalyst;
(vi) providing additional co-ligand to interact with the perfluorinated SABRE catalyst to reactivate the perfluorinated SABRE catalyst;
(vii) combining the reactivated perfluorinated SABRE catalyst with additional parahydrogen and additional substrate comprising a ½ spin nucleus or nuclei in the solvent to obtain a second reaction mixture; and
(viii) hyperpolarizing the second reaction mixture obtained in (vii) by exposing the mixture to a magnetic field or by radiofrequency excitation to obtain additional hyperpolarized active perfluorinated SABRE catalyst-substrate and/or additional hyperpolarized substrate.

2. The method of claim 1, wherein the substrate comprises ¹H, ¹³C, ¹⁵N, ¹⁹F, ³¹p ²⁹Si, or a combination thereof.

3. The method of claim 2, wherein the substrate further comprises ²D.

4. The method of claim 1, wherein the co-ligand is a compound containing one or more sulfoxide groups, thioester groups, phosphine groups, amine groups, CO groups, isonitrile groups, nitrogen-containing heterocyclic groups, or a combination thereof.

5. The method of claim 1, wherein the solvent comprises water, methanol, ethanol, a fluorous solvent, or a mixture thereof.

6. The method of claim 1, wherein the solvent comprises a perfluoroalkane, diethyl ether, a nonafluorobutyl methyl ether, ethyl acetate, perfluorobutyl methyl ether, a fluorocarbon derivative of THF FC 75, a decafluoromethoxy trifluoromethyl pentane, a hexafluoro propanol, a perfluoromethyl cyclohexane, and a combination thereof.

7. The method of claim 1, wherein the solvent comprises water, methanol, ethanol, a nonafluorobutyl methyl ether, deuterated variants thereof, or a mixture thereof.

8. The method of claim 1, wherein the solvent is deuterated.

9. The method of claim 1, wherein the co-ligand is dimethyl sulfoxide.

10. The method of claim 1, wherein the co-ligand is fluorinated.

11. The method of claim 10, wherein the co-ligand is phenyl trifluoromethyl sulfoxide, 2,2,2-trifluoroethyl methane sulfonate, or di-2,2,2-trifluoroethyl sulfoxide.

12. The method of claim 1, wherein the substrate is selected from ketoglutarate, ketoisocaproate, pyruvate, N-acetyl cysteine, and salts or esters thereof.

13. The method of claim 1, wherein the substrate is selected from 1-¹³C-ketoglutarate, 1-¹³C-5-¹²C-ketoglutarate, 1-¹³C-pyruvate, 1-¹³C-N-acetyl cysteine, ¹⁵N₂-isoniazid (or pyridyl-4-carbo-bis-¹⁵N₂-hydrazide), ¹³C₂,¹⁵N₃-metronidazole, ¹⁵N₂-1-aminoisoquinoline (1-AIQ), deuterated versions thereof, and salts thereof.

14. The method of claim 1, wherein the substrate is of Formula (II): 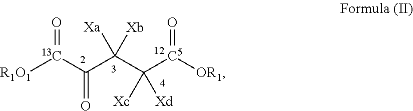
wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, provided that at least one of Xa, Xb, Xc, and Xd is deuterium,
or a pharmaceutically acceptable salt thereof.

15. The method of claim 1, wherein NHC is a 5-membered N-heterocyclic carbenyl group.

16. The method of claim 15, wherein the 5-membered N-heterocyclic carbenyl group is imidazole-based, imidazoline-based, or thiazole-based.

17. The method of claim 1, wherein the perfluorinated ligand is of Formula (Ia) or (Ib): 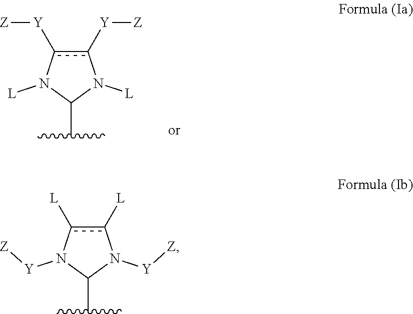
or a salt thereof, and wherein
each L independently is hydrogen, adamantyl, a substituted or unsubstituted aromatic group, or a substituted or unsubstituted heteroaromatic group,
each Y independently is a bond or a spacer group,
each Z independently is a perfluorinated tag, [symbol]
is a single bond or a double bond, and [symbol]
represents the bond to the d-block element via the carbene.

18. The method of claim 1, wherein the perfluorinated ligand is of Formula (Ic) or (Id): 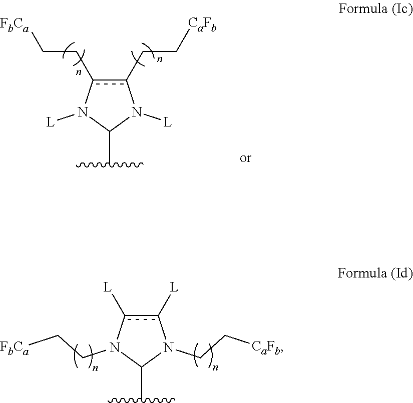
or a salt thereof, and wherein
each L independently is hydrogen, adamantyl, a substituted or unsubstituted aromatic group, or a substituted or unsubstituted heteroaromatic group,
a is 4 to 20,
b=2a+1 or b=a−1,
each n independently is an integer from 0 to 4, [symbol]
a single bond or a double bond, and [symbol]
represents the bond to the d-block element via the carbene.

19. The method of claim 1, wherein the perfluorinated ligand is of Formula (Ie) or (If): 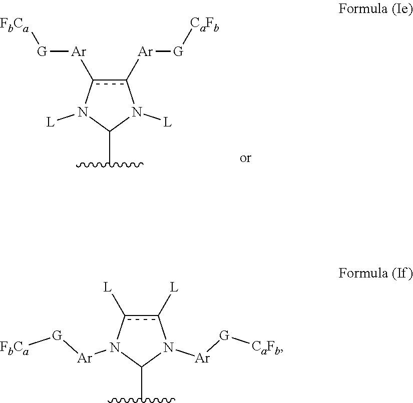
or a salt thereof, and wherein
each L independently is hydrogen, adamantyl, a substituted or unsubstituted aromatic group, or a substituted or unsubstituted heteroaromatic group,
each Ar independently is a substituted or unsubstituted aromatic group, or a substituted or unsubstituted heteroaromatic group,
each G independently is a bond, C₁₋₆ alkyl, C₁₋₆ alkenyl, or C₁₋₆ heteroalkyl,
a is 4 to 20,
b=2a+1 or b=a−1, [symbol]
is a single bond or a double bond, and [symbol]
represents the bond to the d-block element via the carbene.

20. The method of claim 1, where each L independently is hydrogen, adamantyl, 2-methylphenyl, 3-methylphenyl, 4-methylphenyl, 2,4-dimethylphenyl, 2,5-dimethylphenyl, 2,6-dimethylphenyl, 3,5-dimethylphenyl, 2,4,6-trimethylphenyl, 2-ethylphenyl, 3-ethylphenyl, 4-ethylphenyl, 2,4-diethylphenyl, 2,5-diethylphenyl, 2,6-diethylphenyl, 3,5-diethylphenyl, 2,4,6-triethylphenyl, 2-npropylphenyl, 3-npropylphenyl, 4-npropylphenyl, 2,4-di-npropylphenyl, 2,5-di-npropylphenyl, 2,6-di-npropylphenyl, 3,5-di-npropylphenyl, 2,4,6-tri-npropylphenyl, 2-isopropylphenyl, 3-isopropylphenyl, 4-isopropylphenyl, 2,4-di-isopropylphenyl, 2,5-di-isopropylphenyl, 2,6-di-isopropylphenyl, 3,5-di-isopropylphenyl, 2,4,6-tri-isopropylphenyl, 2-isobutylphenyl, 3-isobutylphenyl, 4-isobutylphenyl, 2,4-di-isobutylphenyl, 2,5-di-isobutylphenyl, 2,6-di-isobutylphenyl, 3,5-di-isobutylphenyl, 2,4,6-tri-isobutylphenyl, 2-secbutylphenyl, 3-secbutylphenyl, 4-secbutylphenyl, 2,4-di-secbutylphenyl, 2,5-di-secbutylphenyl, 2,6-di-secbutylphenyl, 3,5-di-secbutylphenyl, 2,4,6-tri-secbutylphenyl, 2-tbutylphenyl, 3-tbutylphenyl, 4-tbutylphenyl, 2,4-di-tbutylphenyl, 2,5-di-tbutylphenyl, 2,6-di-tbutylphenyl, 3,5-di-tbutylphenyl, 2,4,6-tri-tbutylphenyl, 2-cyclohexylphenyl, 3-cyclohexylphenyl, 4-cyclohexylphenyl, 2,4-di-cyclohexylphenyl, 2,5-di-cyclohexylphenyl, 2,6-di-cyclohexylphenyl, 3,5-di-cyclohexylphenyl, or 2,4,6-tri-cyclohexylphenyl.

21. The method of claim 1, wherein each Y independently is a bond, a substituted or unsubstituted C₁₋₁₀ alkyl group, a substituted or unsubstituted C₂₋₁₀ alkenyl group, a substituted or unsubstituted C₂₋₁₀ alkynyl group, a substituted or unsubstituted C₁₋₁₀ heteroalkyl group, a substituted or unsubstituted C₃₋₆ cycloalkyl group, a substituted or unsubstituted C₃₋₆ heterocycloalkyl group, a substituted or unsubstituted aryl group, a substituted or unsubstituted heteroaryl group, a substituted or unsubstituted alkaryl group, a substituted or unsubstituted arylalkyl group, or a linear or branched alkyleneoxy group.

22. The method of claim 1, wherein the perfluorinated tag is a perfluorinated C₃₋₆₀ group comprising only carbon and fluorine atoms.

23. The method of claim 1, wherein the perfluorinated ligand is 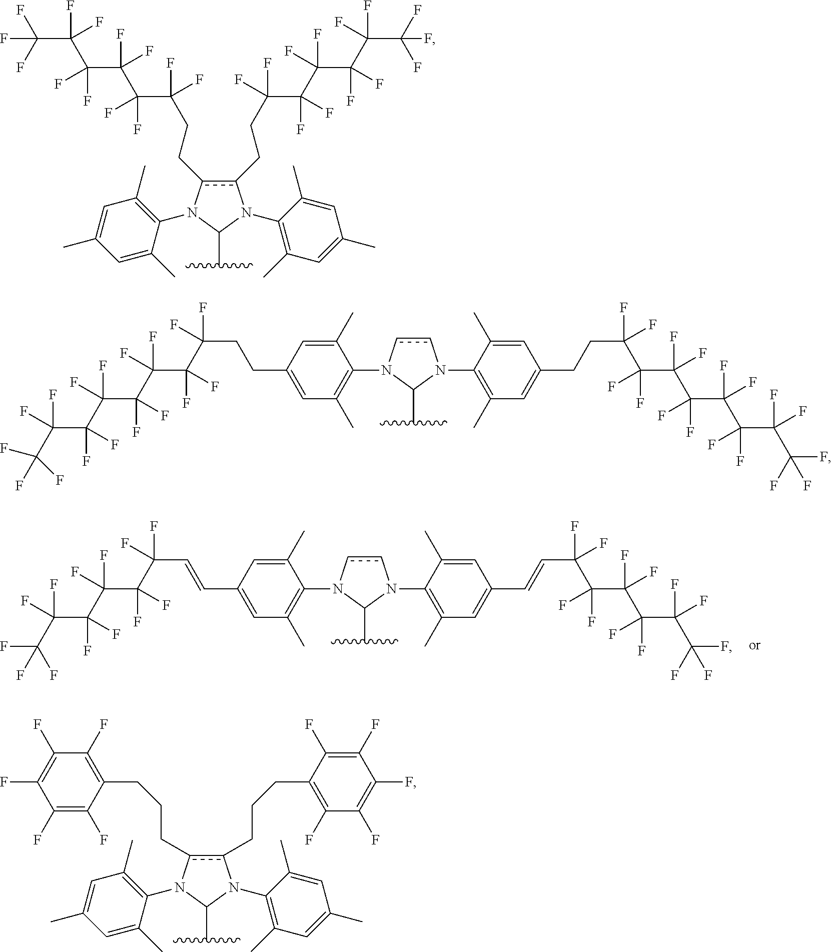
or a salt thereof, and wherein [symbol]
is a single bond or a double bond, and [symbol]
represents the bond to the d-block element via the carbene.

24. The method of claim 1, wherein the d-block element is Co, Rh, Ir, Ru, Pd, Pt, or Mt.

25. A kit comprising:
(i) a perfluorinated SABRE catalyst comprising a d-block element and a perfluorinated ligand, wherein the perfluorinated ligand is of Formula (I):
[Lₘ-(NHC)—(Y—Z)_{q}] Formula (I),
or a salt thereof, and wherein
each L is independently selected from hydrogen, adamantyl, a substituted or unsubstituted aromatic group, or a substituted or unsubstituted heteroaromatic group,
NHC is a 4 to 7-membered N-heterocyclic carbenyl group where NHC is bound to the d-block element via a carbene,
each Y is independently selected from a bond or a spacer group,
each Z is a perfluorinated tag,
m is an integer from 1 to 4, and
q is an integer from 1 to 3;
(ii) a co-ligand;
(iii) a solvent; and
(iv) optionally a substrate.
